(19) United States
(12) Patent Application Publication
(10) Pub. No.: US 2025/0277200 A1
(43) Pub. Date: Sep. 4, 2025

(54) ENGINEERED CPN1 CONSTRUCTS AND VARIANTS

(71) Applicant: Vertex Pharmaceuticals Incorporated, Boston, MA (US)

(72) Inventors: Aaron AGUHOB, South San Francisco, CA (US); Natacha LE MOAN, San Francisco, CA (US); Grant E. BLOUSE, Burlingame, CA (US); Arzu SANDIKCI, Berkeley, CA (US); Ryan PAVLOVICZ, Seattle, WA (US); Amanda LOSHBAUGH, Seattle, WA (US); Yifan SONG, Seattle, WA (US); Timothy MYLES, South San Francisco, CA (US)

(21) Appl. No.: 18/274,430

(22) PCT Filed: Jan. 27, 2022
(86) PCT No.: PCT/US2022/014162

§ 371 Date: Sep. 10, 2024

Related U.S. Application Data
(60) Provisional application No. 63/142,462, filed on Jan. 27, 2021.
(60) Provisional application No. 63/222,929, filed on Jul. 16, 2021.

(51) Int. Cl. C12N 9/48; A61K 38/00

(52) U.S. Cl. CPC C12N 9/485; C12Y 304/17003; A61K 38/00; C07K 2319/31; C07K 2319/50

(57) ABSTRACT

Provided herein are carboxypeptidase N catalytic subunit (CPN1) variants, comprising at least one modification with respect to a wild type carboxypeptidase N1 of the M14 family, wherein the variants have at least one improved characteristic as compared to the wild type CPN1. Also provided herein are fusion constructs comprising CPN1, or variants thereof. Also provided herein are methods of making and using such variants and constructs. The variant and constructs provided herein may be useful for treating diseases or conditions associated with dysregulation of the complement system.

### CROSS REFERENCE TO RELATED APPLICATIONS

This application claims priority to U.S. Provisional Application No. 63/142,462 filed on Jan. 27, 2021, and U.S. Provisional Application No. 63/222,929 filed on Jul. 16, 2021, the contents of which are incorporated herein by reference in their entireties.

### REFERENCE TO SEQUENCE LISTING

An electronic version of the Sequence Listing is filed herewith, the contents of which are incorporated by reference in their entirety. The electronic file was created on Jan. 27, 2022, is 3.29 megabytes in size, and is titled CTBI_005_02WO_SeqList_ST25.txt.

### BACKGROUND

The complement system includes the classical, alternative, and lectin pathways, and is tightly controlled by a number of regulators. One such regulator is carboxypeptidase N (CPN), a plasma enzyme of the metalloprotease class and M14B subfamily. CPN cleaves basic amino acids from the C-terminal end of bioactive peptides and proteins, leading to their inactivation. CPN has a role in preventing the buildup of peptides that are involved in signaling and regulation of inflammation, or controlling blood pressure, thus regulation of the levels of these peptides is necessary. CPN is synthesized in the liver and secreted into the bloodstream in a constitutively active form, while another similar carboxypeptidase, carboxypeptidase B2 (CPB2), circulates in plasma as a zymogen, and requires activation by the thrombin-thrombomodulin complex or plasmin. Normally, CPN circulates in the blood as a hetero-tetramer consisting of two 83 kDa (CPN2) domains, each flanked by a 48 to 55 kDa catalytic (CPN1) domain.

While CPN targets a number of substrates, CPN plays a significant role in complement regulation by targeting C3a and C5a, which are generated during complement activation. C3a stimulates macrophages and is implicated in B cell antibody response. Due to the expression of C3a receptor (C3aR) at the surface of endothelial cells and platelets, C3a can also be involved in platelet function and thrombus formation. C5a is a chemotactic factor for leukocytes and activates neutrophils, basophils and mast cells and therefore can be involved in expansion of T helper type 1 (Th1) cells and suppression of regulatory T cells (Treg). CPN inactivates C3a and C5a by cleaving one or more amino acids from their C-terminal ends. Like CPN, CPB2 also has several physiological substrates, including C3a and C5a. Continuous regulation of C3a and C5a levels is necessary to maintain a pro- and anti-inflammatory balance in the complement system. For example, a genetic deficiency in CPN or CPB2 can result in the exacerbation of pathological symptoms of complement disorders such as hemolytic-uremic syndrome (HUS) and cobra venom factor (CVF) challenge. Provided herein are compositions and methods to address the dysfunction and/or dysregulation in the complement system.

### SUMMARY

In one aspect provided herein are variants of a carboxypeptidase N catalytic subunit (CPN1) comprising at least one modification with respect to a wild type CPN1, wherein the variant has at least one improved characteristic as compared to the wild type CPN1.

In another aspect, provided herein are fusion constructs comprising a carboxypeptidase N catalytic subunit (CPN1) or variant thereof. Exemplary fusion constructs are provided in Table 2 and Table 5.

In another aspect provided herein is a method of treating a disease or condition in a subject in need thereof, comprising administering to the subject any one of the CPN1 variants or fusion constructs of the disclosure.

### BRIEF DESCRIPTION OF THE DRAWINGS

FIG. 1A depicts a schematic diagram of a CPN tetramer made up of two heterodimers, and FIG. 1B depicts a single heterodimer of the tetramer.

FIG. 2 depicts a schematic diagram of CPB2 (TAFI), showing both the zymogen and activated forms.

FIG. 3 depicts schematic diagrams of a wild type CPN with its catalytic subunit (CPN1) alone, with its catalytic subunit (CPN1) plus its regulatory subunit (CPN2), and various exemplary fusion constructs comprising other components.

FIG. 4A depicts schematic diagrams of exemplary fusion constructs of the disclosure comprising various activation peptides to alter the sensitivity of the constructs to certain complement components.

FIGS. 4B-4C depict the structure of a CPN1-CPB2 fusion, showing the CPB2 activation peptide (TAFI activation peptide) masking the catalytic site of CPN1, and a schematic diagram of the fusion, respectively.

FIGS. 4D-4E depict the structure of a CPN1-CPA4 fusion, and a surface representation of the same, respectively.

FIGS. 4F-4G depict the structure of a CPN1-CPA1 fusion, and a surface representation of the same, respectively.

FIG. 5 depicts a general schematic diagram of a screening process for activation peptides (SEQ ID NOs: 45-63), and examples of libraries for the activation peptide screening.

FIGS. 6A-6C depict Coomassie staining from SDS-PAGE analysis showing various fusion constructs (chimeras) expressed in Expi293 cells. FIG. 6D shows Coomassie staining from SDS-PAGE analysis of CPN1-containing fusion construct expression, using a similar process as depicted in FIGS. 6A-6C, with five constructs. FIGS. 6E-6H show exemplary CPN1-containing fusion constructs and their corresponding SDS-PAGE expression analysis. FIGS. 61-6J shows exemplary CPN1-HSA fusion constructs with and without TEV protease cleavage sites (SEQ ID NOs: 64-66), and FIG. 6K shows their corresponding SDS-PAGE expression analysis. FIG. 6L shows SDS-PAGE analysis of an exemplary CPN1-HSA fusion construct in the presence or absence of TEV protease. FIG. 6M shows the results of a peptide-based TAFI activity assay for exemplary CPN1-HSA constructs in the presence or absence of TEV protease. FIGS. 6N-6O shows SDS-PAGE expression analysis of exemplary CPN1-containing constructs. FIGS. 6P-6W shows SDS-PAGE expression analysis of exemplary CPN1-containing constructs.

FIGS. 7A-7C depict various chromatography and SDS-PAGE results from a two-step purification of exemplary fusion constructs. FIGS. 7D-7F show SDS-PAGE and SEC chromatograms for exemplary CPN1 variants purified by affinity purification.

FIGS. 8A-8B depict examples of data obtained from a cell-based screening assay used to evaluate construct activity on C3a and C5a by measuring the level of activation of C3aR and C5aR by C3a and C5a. FIGS. 8C-8D show activities of exemplary fusion constructs in the Dansyl-Ala-Arg Activity assay. FIGS. 8E-8F show activities of exemplary fusion constructs in the Hippuryl-Arg Activity assay.

FIGS. 9A-9B show results of CPN1-HSA efficacy after intravenous administration in an in vivo rodent model of complement activation. FIG. 9A shows Pulmonary Congestion Index (PenH) % change from baseline. FIG. 9B shows leukocyte infiltration in lung 24 hours after CPN1-HSA treatment.

FIGS. 10A-10F show cytokine and chemokine effects after intravenous administration of CPN1-HSA in an in vivo rodent model of complement activation.

FIGS. 11A-11C depict in vivo pharmacokinetics profiles of CPN1-HSA intravenously administered to rats. FIG. 11A depicts PK parameter estimates for three animals assessed. FIG. 11B depicts the PK profiles of three animals analyzed, and the representative PK profile is depicted in FIG. 11C.

FIG. 12 depicts the regimen for determining the pharmacokinetics profile of CPN1-HSA intravenously or subcutaneously administered to cynomolgus macaques. Table 1 (of FIG. 12, not to be confused with the Table 1 of the Detailed Description) summarizes the dose levels of the test articles and vehicles. Table 2 (of FIG. 12, not to be confused with the Table 1 of the Detailed Description) summarizes the blood collection schedule.

FIGS. 13A-13D depict the results of serum stability assays for CPN1-HSA. FIGS. 13A & 13B depict Western blots for two exemplary CPN1-HSA constructs. FIGS. 13C and 13D depict half-life curves for exemplary CPN1-HSA constructs in serum.

FIGS. 14A-14D depict the results of CPN1-HSA stability in plasma isolated from rats following intravenous or subcutaneous administration of CPN1-HSA. FIGS. 14A &14B depict two different rats' plasma samples. FIG. 14C depicts CPN1-HSA stability following subcutaneous injection. FIG. 14D depicts CPN1-HSA stability following intravenous injection of human plasma purified CPN1.

FIG. 15 depicts the specific activity of exemplary fusion constructs as determined through a TAFI assay.

### BRIEF DESCRIPTION OF THE TABLES

The following Tables are provided as a part of the application.
Table 1 depicts the amino acid sequences of exemplary components comprising the constructs of the disclosure.
Table 2 depicts exemplary fusion constructs with components in the N and C termini of the core molecule specified.
Table 3A depicts exemplary modification strings of core molecule hCPN1 (1-398) (SEQ ID NO: 7). Amino acid substitutions, deletions, insertions, etc. are noted in conventional format.
Table 3B depicts exemplary modification strings of core molecule hCPN1 (1-320) (SEQ ID NO: 8). Amino acid substitutions, deletions, insertions, etc. are noted in conventional format.
Table 3C depicts exemplary modification strings of core molecule hCPN1 (1-438) (SEQ ID NO: 6). Amino acid substitutions, deletions, insertions, etc. are noted in conventional format.
Table 4A depicts exemplary modifications of individual residues of core molecule hCPN1 (1-398) (SEQ ID NO: 7). Amino acid substitutions, deletions, insertions, etc. are noted in conventional format.
Table 4B depicts exemplary modifications of individual residues of core molecule hCPN1 (1-320) (SEQ ID NO: 8). Amino acid substitutions, deletions, insertions, etc. are noted in conventional format.
Table 4C depicts exemplary modifications of individual residues of core molecule hCPN1 (1-438) (SEQ ID NO: 6). Amino acid substitutions, deletions, insertions, etc. are noted in conventional format.
Table 5 depicts the signal sequences and SEQ IDs of exemplary fusion constructs of the disclosure. The signal sequences are optional, and useful for the expression the fusion constructs.

### DETAILED DESCRIPTION

The disclosure provides compositions and methods useful for modulating the signaling and regulation of the complement system. Specifically, complement system modulation can be observed by providing variants of carboxypeptidase N catalytic subunit (CPN1), and fusion constructs comprising CPN1, and variants thereof. Provided herein are CPN1 variants, and CPN-1 containing fusion constructs, that are more active, and/or more stable in circulation than wild type CPN. Such modulation can include an increase in cleavage and inactivation of C3a and/or C5a, thus reducing complement-mediated inflammation, and reducing the amplification of the complement pathways. For example, some carboxypeptidase variants can alter levels of regulators within the complement system, which includes C3a or C5a. In some embodiments, the variants and fusion constructs provided herein can act on the classical pathway of the complement system, or on the alternative pathway of the complement system, or on the lectin pathway of the complement system, or on one or more pathways. The disclosure also provides methods of making and using these variants and fusion constructs, for example in treating a disease or condition associated with complement dysregulation, e.g. treating an overactive complement response.

### Carboxypeptidase N Variants

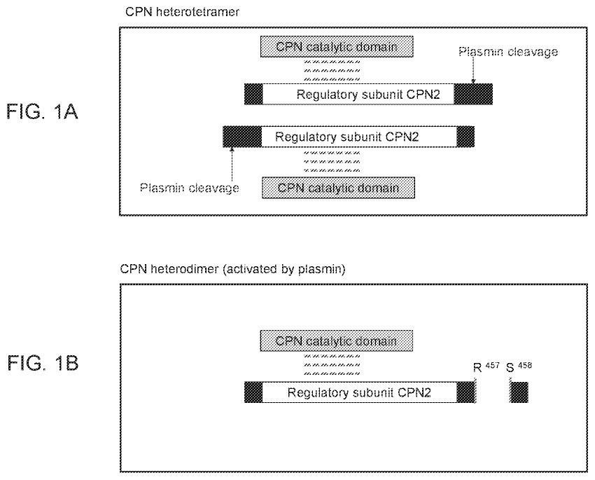

FIG. 1A depicts a schematic diagram of a CPN tetramer made up of two heterodimers. FIG. 1B depicts a single heterodimer of the tetramer. CPN endogenously exists as a tetramer, having two identical catalytic subunits called CPN1 (“CPN catalytic domain” in FIGS. 1A-1B), and two identical regulatory subunits called CPN2 (“regulatory subunit CPN2” in FIGS. 1A-1B) which act to stabilize the CPN1 subunits. Each heterodimer includes a CPN1 domain and a CPN2 domains. CPN1 is also referred to as a CPN catalytic domain herein.

In some embodiments, provided herein are CPN variants, such variants comprise one or more modifications with respect to a wild type CPN, and are referred to herein as “CPN variants.” As used herein, a “modification” to a wild type CPN includes: a deletion of one or more amino acid residues, a deletion of one or more CPN domains, a substitution of one or more amino acid residues in one or more domains, a substitution of one or more CPN domains, an insertion of one or more amino acid residues in one or more domains, an insertion of one or more CPN domains, a swapping of one or more CPN domains, an insertion of one or more domains from a protein or any other component that is not CPN, and a fusion to a protein or component that is not CPN. For example, such components (collectively referred to herein as a “non-CPN domain”) can be, but are not limited to, other members of the carboxypeptidase family such as CPB2, or a half-life extender. Such CPN variants fused to one or more non-CPN domains, such as domains from other members of the carboxypeptidase family, may be referred to herein as “CPN chimeras” or “CPN fusion proteins” or “CPN fusion constructs.”

In some embodiments, provided herein are variants of the CPN catalytic domain (also referred to herein as CPN1), such variants comprise one or more modifications with respect to a wild type CPN1, and are referred to herein as “CPN1 variants.” As used herein, a “modification” to a wild type CPN1 includes one or more of: a deletion of one or more amino acid residues, a substitution of one or more amino acid residues, and an insertion of one or more amino acid residues. As used herein, a “CPN1 variant” is any CPN1-derived polypeptide having a modification to a wild type CPN1. As used herein, a “wild type CPN1” is a naturally-occurring CPN1 that is not a disease-causing CPN1. In some embodiments, the wild type CPN1 is a human CPN1. A CPN1 variant may also be referred to as a CPN variant, that is all CPN1 variants are CPN variants, but the opposite is not the case.

Table 1 provides SEQ ID NO: 6, the full length human wild type CPN1. Table 1 provides the amino acid sequences of human wild-type CPN1 (SEQ ID NO: 1) and CPB2 (SEQ ID NO: 2), inclusive of their signal sequences. The peptide signal sequence of each are indicated in bolded letters. The terms “peptide signal”, “signal peptide”, and “signal sequence” are used interchangeably herein. The underlined residues and slash in the amino acid sequence of CPB2 presented in SEQ ID NO: 2 shows the thrombin-thrombomodulin (T-TB) cleavage sequence. Also provided in Table 1 is an exemplary CPN1 variant fused to an activation peptide T-TB from CPB2, shown in SEQ ID NO: 3.

Table 1 provides a number of human CPN1 amino acid sequences, some of which are truncation variants of full length human wild type CPN1 of SEQ ID NO: 6. The sequences of Table 1 include: SEQ ID NO: 1, which constitutes full length human wild type CPN1 with a signal peptide (referred to herein as hCPN1 (1-438) with a signal peptide); SEQ ID NO: 6, which constitutes full length human wild type CPN1, (referred to herein as hCPN1 (1-438); SEQ ID NO: 7, which constitutes the N-terminal amino acids 1-398 of SEQ ID NO: 6 (referred to herein as hCPN1 (1-398), a CPN1 truncation variant); SEQ ID NO: 11, which constitutes the N-terminal amino acids 1-397 of SEQ ID NO: 6 (referred to herein as hCPN1 (1-397), a CPN1 truncation variant); SEQ ID NO: 12, which constitutes the N-terminal amino acids 1-396 of SEQ ID NO: 6 (referred to herein as hCPN1 (1-396), a CPN1 truncation variant); SEQ ID NO: 8, which constitutes the N-terminal amino acids 1-320 of SEQ ID NO: 6 (referred to herein as hCPN1 (1-320), a CPN1 truncation variant). One or more of these hCPN1 sequences are further modified to generate the CPN1 variants of the disclosure, as exemplified in Tables 3A-4C and 4A-4C. These hCPN1 sequences, including variants thereof, are also used as core molecules for the fusion constructs of the disclosure, as provided in Tables 2 and 5.

In some embodiments, the CPN1 variants provided herein are useful for treatment of a subject in need thereof. As used herein, the terms “patient” or “subject” refer to any vertebrate including, without limitation, humans and other primates (e.g., chimpanzees, cynomolgus monkeys, and other apes and monkey species), farm animals (e.g., cattle, sheep, pigs, goats and horses), domestic mammals (e.g., dogs and cats), laboratory animals (e.g., rabbits, rodents such as mice, rats, and guinea pigs), and birds (e.g., domestic, wild and game birds such as chickens, turkeys and other gallinaceous birds, ducks, geese, and the like). In some embodiments, the subject is a mammal. In exemplary embodiments, the subject is a human.

In some embodiments, provided are CPN2 variants; a CPN2 variant may comprise one or more modifications with respect to hCPN1 (1-456), SEQ ID NO: 10.

In some embodiments, the CPN1 variant of the disclosure comprises at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 6.

In some embodiments, the CPN1 variant of the disclosure comprises at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 7.

In some embodiments, the CPN1 variant of the disclosure comprises at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 8.

In some embodiments, the CPN1 variant of the disclosure comprises at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 11.

In some embodiments, the CPN1 variant of the disclosure comprises at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 12.

In some embodiments, the CPN1 variant of the disclosure comprises SEQ ID NO: 6, SEQ ID NO: 7, SEQ ID NO: 8, SEQ ID NO: 11, or SEQ ID NO:12, comprising one of the modification strings selected from the group consisting of the modification strings provided in Table 4A, Table 4B, and Table 4C.

In some embodiments, the CPN1 variant of the disclosure comprises SEQ ID NO: 6, SEQ ID NO: 7, SEQ ID NO: 8, SEQ ID NO: 11, or SEQ ID NO:12, comprising one of the modification strings selected from the group consisting of the modification strings provided in Table 3A, Table 3B, and Table 3C.

In some embodiments, a CPN1 variant comprise one or more modifications with respect to hCPN1 (1-438), SEQ ID NO: 6. Table 4C of the disclosure provides exemplary modifications of hCPN1 (1-438), SEQ ID NO: 6, listed singly. Accordingly, a CPN1 variant of the disclosure may comprise one or more of the modifications provided in Table 4C. Table 3C of the disclosure provides exemplary modification strings of hCPN1 (1-438), SEQ ID NO: 6. Accordingly, a CPN1 variant of the disclosure may comprise one of the modification strings provided in Table 3C.

In some embodiments, a CPN1 variant comprise one or more modifications with respect to hCPN1 (1-398), SEQ ID NO: 7. Table 4A of the disclosure provides exemplary modifications of hCPN1 (1-398), SEQ ID NO: 7 listed singly. Accordingly, a CPN1 variant of the disclosure may comprise one or more of the modifications provided in Table 4A. Table 3A of the disclosure provides exemplary modification strings of hCPN1 (1-398), SEQ ID NO: 7. Accordingly, a CPN1 variant of the disclosure may comprise one of the modification strings provided in Table 3A.

In some embodiments, a CPN1 variant comprise one or more modifications with respect to hCPN1 (1-397), SEQ ID NO: 11.

In some embodiments, a CPN1 variant comprise one or more modifications with respect to hCPN1 (1-396), SEQ ID NO: 12.

In some embodiments, a CPN1 variant is hCPN1 (1-320) (also referred to herein as hCPN1 (1-320) Delta TT), SEQ ID NO: 8. The amino acid sequence of hCPN1 (1-320), SEQ ID NO: 8, is a C-terminal truncation of the transthyretin (TT) domain of hCPN1 (1-398), SEQ ID NO: 7. In some embodiments, a CPN1 variant comprise one or more modifications with respect to hCPN1 (1-320), SEQ ID NO: 8. Table 4B of the disclosure provides exemplary modifications of hCPN1 (1-320), SEQ ID NO: 8, listed singly. Accordingly, a CPN1 variant of the disclosure may comprise one or more of the modifications provided in Table 4B. Table 3B of the disclosure provides exemplary modification strings of hCPN1 (1-320), SEQ ID NO: 8. Accordingly, a CPN1 variant of the disclosure may comprise one of the modification strings provided in Table 3B.

In some embodiments, the CPN1 variants provided herein are in an active form. In other embodiments the CPN1 variants are provided in an inactive, zymogen form.

### Improved Characteristics of the CPN1 Variants

The CPN1 variants provided herein may modulate activity of the complement system, and have at least one improved characteristic as compared to a wild type CPN1. In some embodiments, the CPN1 variants provided herein have at least one improved characteristic as compared to the wild type CPN1, wherein the wild type CPN1 is a human CPN1. In some embodiments, the improved characteristic includes, but is not limited to, an increase or a decrease in any one or more of: half-life, activity, potency, affinity for one or more substrates, sensitivity, cofactor affinity, stability, and catalytic capability.

In some embodiments, the CPN1 variants do not require a CPN2 for activity. In some embodiments, the CPN1 variants exhibit a higher affinity for C3a, C5a, or both C3a and C5a. In some embodiments the CPN1 variants are part of a fusion construct comprising additional N-terminal and C-terminal domains. In some embodiments, the CPN1 variants are non-immunogenic.

In some embodiments, the at least one improved characteristic comprises an increase in affinity for one or more substrates, wherein at least one substrate is C3a. In some embodiments, the at least one improved characteristic comprises an increase in affinity for one or more substrates, wherein at least one substrate is C5a. In some embodiments, the CPN1 variants provided herein are inactive towards the C5adesArg and C3aDesArg.

In some embodiments, the increase in activity of a CPN1 variant of the disclosure, as compared to a wild type CPN1, comprises an increase in the cleavage of C3a and/or C5a.

In some embodiments, the increase in activity of a CPN1 variant of the disclosure, as compared to a wild type CPN1, comprises an increased k_{cat}/K_{M} (M⁻¹ s⁻¹) for cleavage of C3a and/or C5a.

In some embodiments, the increased k_{cat}/K_{M} (M⁻¹ s⁻¹) for cleavage of C3a and/or C5a exhibited by a CPN1 variant is about 2-fold, about 3-fold, about 4-fold, about 5-fold, about 6-fold, about 7-fold, about 8-fold, about 9-fold, about 10-fold greater than that of the wild type CPN1.

In some embodiments, the increase in activity of a CPN1 variant of the disclosure, as compared to a wild type CPN1, comprises an increase in k_{cat} with a decrease in K_{M}. Generally, an increase in k_{cat} with a decrease in K_{M} is an increase in affinity, and therefore, in some embodiments, the increase in activity comprises an increase in affinity for a substrate.

In some embodiments, the increase in activity of a CPN1 variant of the disclosure, as compared to a wild type CPN1, comprises decrease in K_{D} (nM) value for cleavage of C3a and/or C5a. Generally, a decrease in K_{D} value is an increase in affinity, and therefore, in some embodiments, the increase in activity comprises a decrease in K_{D} value. In some embodiments, the decreased K_{D} (nM) value is about 2-fold, about 3-fold, about 4-fold, about 5-fold, about 6-fold, about 7-fold, about 8-fold, about 9-fold, about 10-fold less than that of the wild type CPN1.

In some embodiments, the increase in activity of a CPN1 variant of the disclosure, as compared to a wild type CPN1, comprises a decrease in EC₅₀ (nM) value for cleavage of C3a and/or C5a, compared to the wild type CPN. Generally, a decrease in EC₅₀ is an increase in affinity, and therefore, in some embodiments, the increase in activity comprises a decrease in EC₅₀.

In some embodiments, the decreased EC₅₀ (nM) value for cleavage of C3a and/or C5a is ranges from about 0.1 nM to about 20 nM. In some embodiments, the value is about 20 nM, or is lower than about 20 nM, e.g. about 10 nM, or about 15 nM, In some embodiments, the decreased EC₅₀ value for cleavage is about 1 nM, about 0.1 nM, or less than about 0.1 nM.

In some embodiments, the improved characteristic of a CPN1 variant of the disclosure, as compared to a wild type CPN1, is an increased half-life, wherein the increased half-life is half-life in plasma. In some embodiments, the increased half-life in plasma is greater than about 24 hours. In some embodiments, the increased half-life in plasma is about 48 hours, about 50 hours, about 60 hours, about 70 hours, about 80 hours, about 90 hours, about 100 hours, or about 150 hours. In some embodiments, the increased half-life in plasma is from about 70 hours to about 150 hours.

In some embodiments, the improved characteristic of a CPN1 variant of the disclosure, as compared to a wild type CPN1, is an increased sensitivity for a substate, wherein the increased sensitivity comprises increased in catalytic activity upon complement activation.

The CPN1 variants can be generated by introducing one or more modifications to a wild type CPN1. In some embodiments, the CPN1 variant comprises at least one modification corresponding to a wild type non-human CPN1.

In some embodiments, a modification to the amino acid sequence as set forth in SEQ ID NO: 6 can increase affinity of a CPN1 variant for C3a and/or C5a as compared to a CPN that is not modified. In some embodiments, a modification to the amino acid sequence as set forth in SEQ ID NO: 6 includes a truncation of a loop domain.

In some embodiments, the CPN1 variant comprises the deletion of one or more amino acid residues, e.g., a truncated CPN1 variant of the disclosure may be selected from the group consisting of SEQ ID NO: 7, SEQ ID NO: 8, SEQ ID NO: 11, and SEQ ID NO: 12. These truncated variants can further serve as base molecules for fusion constructions comprising CPN1 variants of the disclosure.

### Fusion Constructs

Provided herein are fusion constructs comprising wild type CPN1 or any of the CPN1 variants described herein.

In some embodiments, provided herein are CPN1-containing fusion constructs (fusions to other proteins or components), referred to herein as “CPN1 constructs”. In some embodiments, CPN1 constructs comprise a fusion to a protein or component that is from CPN (such as CPN2). In some embodiments, CPN1 constructs comprise a fusion to a protein or component that is not from CPN. For example, such components can be, but are not limited to, other members of the carboxypeptidase family such as CPB2, or a half-life extenders (e.g. albumin).

In some embodiments, hCPN1 (1-438), SEQ ID NO: 6 can be considered a core CPN1 molecule based upon which fusion constructs are generated. In some embodiments, hCPN1 (1-398), SEQ ID NO: 7 can be considered a core CPN1 molecule based upon which fusion constructs are generated. In some embodiments, hCPN1 (1-320), SEQ ID NO: 8 can be considered a core CPN1 molecule based upon which fusion constructs are generated.

In some embodiments, the fusion construct comprises hCPN1 (1-398), SEQ ID NO: 7. In some embodiments, the fusion construct comprises hCPN1 (1-320), SEQ ID NO: 8. In some embodiments, the fusion construct comprises hCPN1 (1-396), SEQ ID NO: 12. In some embodiments, the fusion construct comprises hCPN1 (1-397), SEQ ID NO: 11. In some embodiments, the fusion construct comprises hCPN1 (1-438), SEQ ID NO: 6. In some embodiments, the fusion construct comprises hCPN2 (1-456), SEQ ID NO: 14. In some embodiments, the fusion construct comprises hCPN2 (1-524), SEQ ID NO: 13.

Table 1 provides exemplary components of certain fusion constructs of the disclosure. Table 2 lists exemplary CPN1-containing fusion constructs of the disclosure and their amino acid sequences. In some embodiments the construct comprises one or more of a signal sequence, N-terminal fusion partner 2, N-terminal linker 2, N-terminal fusion partner 1, N-terminal linker 1, a core CPN molecule (such as hCPN1 (1-398), SEQ ID NO: 7, or hCPN1 (1-320), SEQ ID NO: 8), C-terminal linker 1, C-terminal fusion partner, C-terminal linker 2, C-terminal fusion partner 2.

In some embodiments the fusion construct comprises one or more of Human Serum Albumin (HSA), the signal sequence of HSA, the signal sequence of Azurocidin, the signal sequence of interleukin 2, the signal sequence of immunoglobin G, the activation peptide of CPB2 (the N-terminal 96 amino acids of CPB2 protease), Small Ubiquitin Modifying enzyme (SUMO), 10 histidine residues followed by SUMO, Tobacco Etch Virus (TEV) protease cleavage site, linkers consisting of amino acids (such as repeating GS) of various lengths (such as 8, 12, or 20 amino acid residues), His-tag, Fc region (the fragment crystallizable region is the tail region of an antibody that interacts with cell surface receptors called Fc receptors and some proteins of the complement system), mutations introduced in the Fc region (such as no 1st DK LALA-PG), Xa (factor Xa protease cleavage site), mammalian maltose binding protein (mMBP), CPB2, and/or CPN2. To the extent certain abbreviations are provided herein, including the drawings, reference is made to the abbreviations in Table 1.

Table 5 provides the full amino acid sequences of exemplary fusion constructs of the disclosure. Accordingly, in some embodiments, provided herein is a CPN1 fusion construct selected from any of those presented in Table 5. Also provided in Table 5 are optional signal sequence peptides that may be used for the expression of the fusion constructs presented therein.

In some embodiments the activation peptide of CPB2 (the N-terminal 96 amino acids of CPB2 protease) may be mutated to improve binding with CPN1, and is included in a fusion construct of the disclosure.

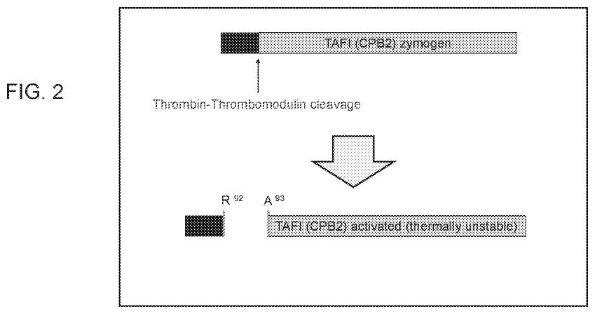

FIG. 2 depicts a schematic diagram of CPB2 (also known as carboxypeptidase U (CPU), plasma carboxypeptidase B (pCPB) or thrombin-activatable fibrinolysis inhibitor (TAFI)), another member of the carboxypeptidase M14 family, which has similar substrates as CPN1. Because CPB2 is secreted as a zymogen in circulation, its domains may be useful in the modification of CPN1 to generate CPN1 zymogen constructs. CPB2 includes an activation peptide (cleavable substrate) and is endogenously activated by thrombin-thrombomodulin or plasmin but is also sensitive to mannan-binding lectin serine protease 1 (MASP1) cleavage in vitro.

As discussed above, it should be understood that a CPN1 variant may also be fused with another component, such as a half-life extender or a portion of another carboxypeptidase providing stability in circulation, as will be further discussed herein.

The fusion constructs of the disclosure may be engineered to display an increase in sensitivity comprises an increase in the sensitivity to any one or more of: Mannan-binding lectin serine protease 1 (MASP1), Mannan-binding lectin-associated serine protease 3 (MASP3), Factor D, methyl-accepting chemotaxis protein (mCPA3) secreted during mast cell degranulation, and cathepsin G secreted during neutrophil degranulation. In some embodiments, the decrease in sensitivity comprises decrease in thrombin-thrombomodulin.

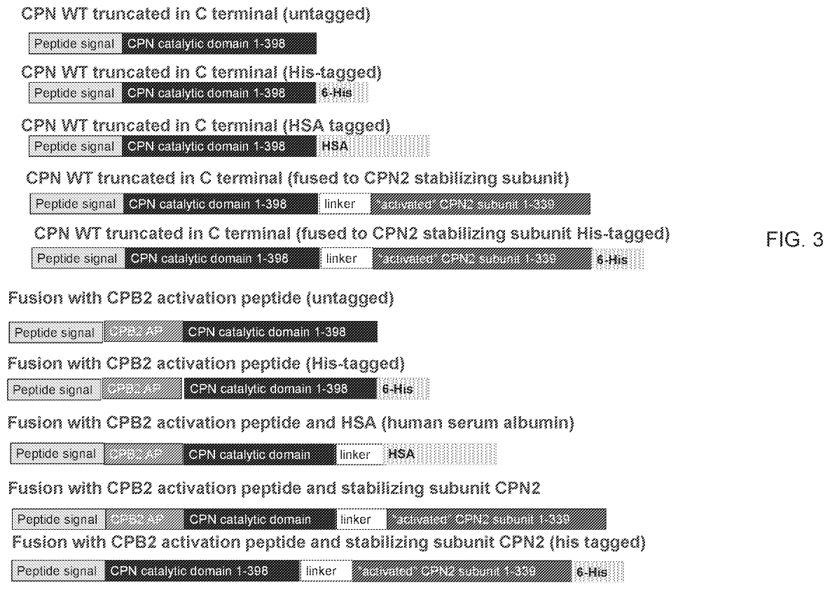

In some embodiments, the fusion constructs provided herein comprise one or more of domains of CPN selected from: a first peptide signal, a catalytic domain (CPN1) or a portion of CPN1, an activation peptide sensitive to complement activation, and a regulatory subunit (CPN2) or a portion of CPN2. FIG. 3 depicts schematic diagrams of a number of exemplary fusion constructs, e.g. a CPN1 with its catalytic subunit alone, a CPN1 with its catalytic subunit plus its regulatory subunit. FIG. 3 also includes a schematic diagram of a CPB2 having a signal peptide (“peptide signal” in FIG. 3 schematics), an activation peptide (AP) domain, and a catalytic domain. By way of example, a fusion construct can include an activation peptide or a portion of the activation peptide from CPB2, and in some embodiments, can be expressed as a single heterodimer.

It should be understood that, while exemplary fusion constructs disclosed herein can include an activation peptide, any of the fusion constructs disclosed herein can be provided optionally without an activation peptide, or optionally with a first and a second activation peptide, or optionally with a single activation peptide.

Exemplary fusion constructs shown as schematic diagrams in FIG. 3 include:
(a) a fusion construct comprising a peptide signal, with an activation peptide from CPB2, a CPN1 domain, with a deletion of its CPN2;
(b) a fusion construct comprising a first peptide signal and CPN1 domain, and with an activation peptide from CPB2, and expressed as a heterodimer, also comprising a second peptide signal and its CPN2 domain;
(c) a fusion construct comprising a peptide signal, an activation peptide from CPB2, a CPN1 domain, a linker, and a CPN2 domain;
(d) a fusion construct comprising a peptide signal, an activation peptide from CPB2, a CPN1 domain, a linker, and a half-life extender (human serum albumin shown as an exemplary half-life extender).

Further, any of the fusion construct provided herein can be HSA-tagged, or His-tagged, as is also shown in FIG. 3 as examples.

In some embodiments, a fusion construct comprises at least one non-CPN domain or component. In such embodiments, the insertion of the at least one non-CPN domain of component can help to stabilize the CPN1 domain, such that the deletion of the CPN2 domain does not reduce the clearance of the CPN1 domain from plasma, for example.

In some embodiments, the at least one non-CPN domain or component comprises at least one domain of carboxypeptidase B2 (CPB2). In some embodiments, the CPB2 is a human CPB2, SEQ ID NO: 2. In some embodiments, the at least one domain of CPB2 comprises an activation peptide.

In some embodiments, the fusion construct comprises at least one modification corresponding to a wild type CPN1 comprising the amino acid sequence as set forth in SEQ ID NO: 3.

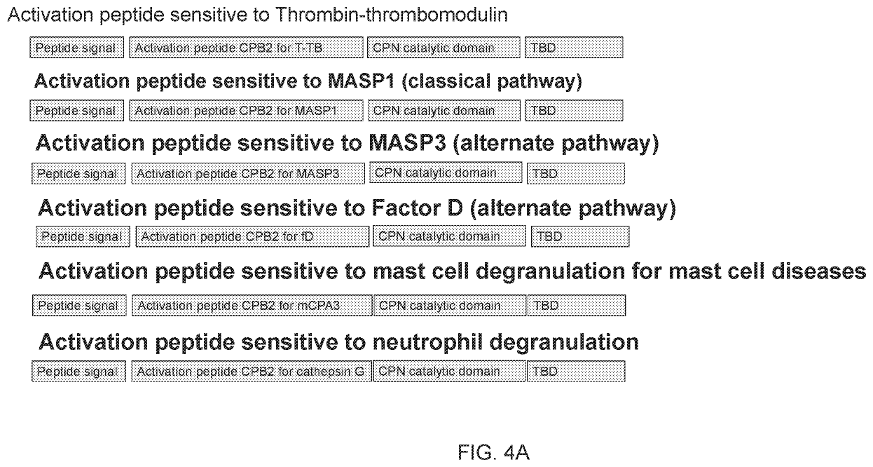

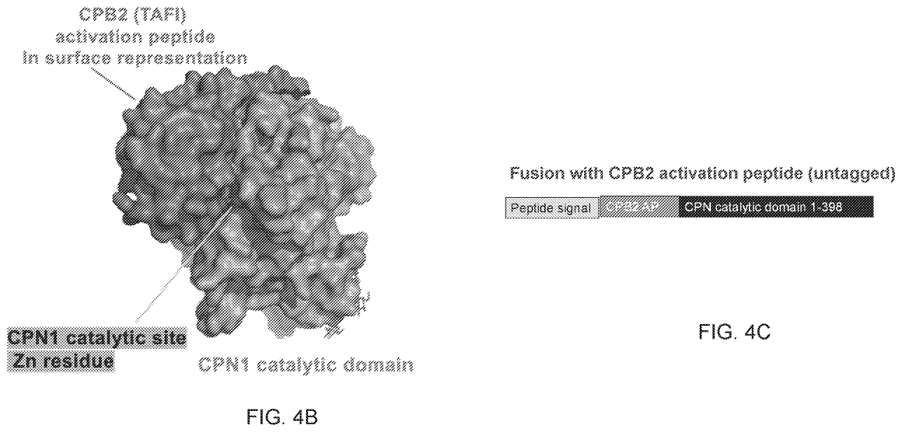

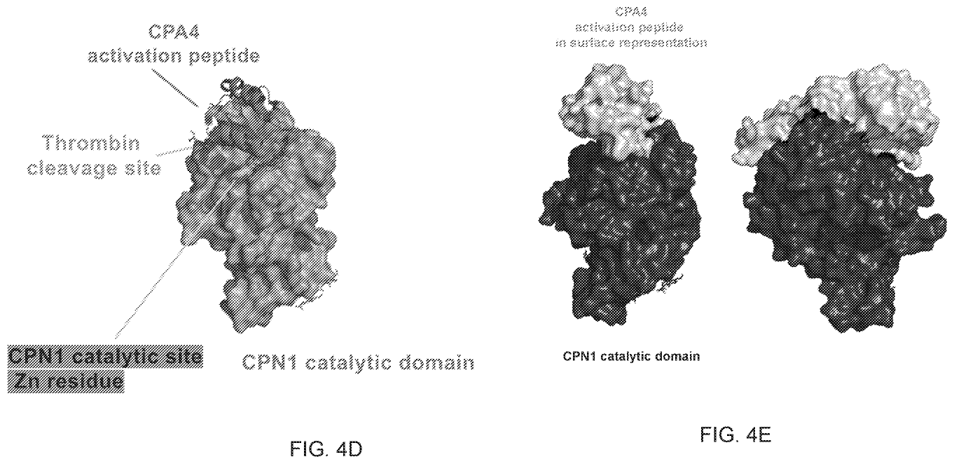

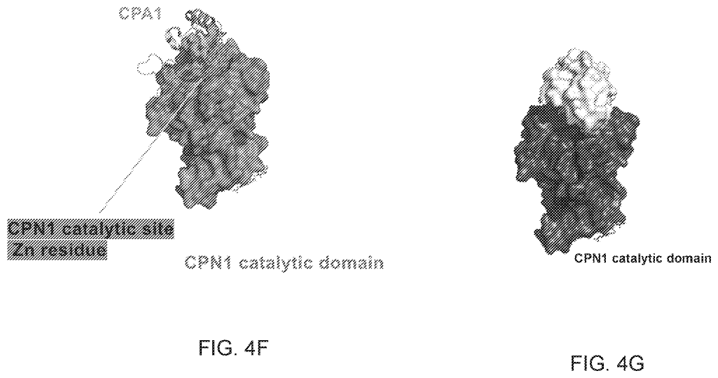

FIG. 4A depicts schematic diagrams of fusion constructs comprising various activation peptides to alter the sensitivity to certain complement components. In some embodiments, the activation peptide increases sensitivity of the fusion construct for any one or more of: MASP1, MASP3, Factor D, mCPA3, or cathepsin G. By way of example, an increased sensitivity for select complement components can direct activity of the fusion construct in the classical, alternate, and lectin pathways. In some embodiments, the fusion construct is not activated by thrombin-thrombomodulin. Exemplary fusion constructs comprising an activation peptide from CPB2 shown as schematic diagrams in FIG. 4A include the activation peptides for the following:
(a) an activation peptide for thrombin-thrombomodulin (T-TB), which can be used as a starting point for generation of peptide libraries that are more sensitive to MASP1, MASP3, Factor D, mCPA3, or cathepsin G, and less sensitive to T-TB;
(b) an activation peptide for MASP1, which can increase sensitivity to MASP1, within the classical pathway of the complement system;
(c) an activation peptide for MASP3, which can increase sensitivity to MASP3, within the alternate pathway of the complement system;
(d) an activation peptide for Factor D, which can increase sensitivity to Factor D, within the alternate pathway of the complement system;
(e) an activation peptide for mCPA3, which can increase sensitivity to mast cell degranulation, for mast cell diseases;
(f) an activation peptide for cathepsin G, which can increase sensitivity to neutrophil degranulation, within the classical, alternate, and/or lectin pathways of the complement system.

The fusion constructs may be designed with an activation peptide sensitive to neutrophil or mast cell activation. Neutrophil-sensitive activation peptide can allow targeting ANCA and a mast cell-activation peptide can allow targeting skin HS and a broad range of mast cell related disorders.

As noted above, also provided herein are fusion constructs comprise one or more domains of a non-CPN component, such as a non-CPN carboxypeptidase. In some embodiments, the activation peptide of a fusion construct masks the catalytic site of CPN1. An exemplary CPN fusion construct with this property is a CPN1-CPB2 chimera. FIGS. 4B-4C depict the structure of a CPN1-CPB2 chimera, showing the CPB2 activation peptide (TAFI activation peptide) masking the catalytic site of CPN1, and a schematic diagram of the fusion construct, respectively. The inclusion of a mask effectively allows the CPN1 containing fusion construct to be delivered in a zymogen format.

Another example of such a fusion construct is a fusion of CPN1 and carboxypeptidase A4 (CPA4). FIGS. 4D-4E depict the structure of a CPN1-CPA4 fusion construct, and a surface representation of the same, respectively. These figures show that the activation peptide from CPA4 masks the catalytic site of CPN1.

Another example of such a fusion construct is a fusion of CPN1 and carboxypeptidase A1 (CPA1). FIGS. 4F-4G depict the structure of a CPN1-CPA1 fusion, and a surface representation of the same, respectively. These figures show that the activation peptide from CPA1 masks the catalytic site of CPN1.

In some embodiments, the activation peptide increases sensitivity of the fusion construct for any one or more of: mast cell degranulation, neutrophil degranulation, and inflammatory cell activation. In some embodiments, the activation peptide increases sensitivity of the fusion construct to complement activation.

In some embodiments, fusion construct is a fusion and comprises at least one CPB2 domain which comprises any one or more of: a CPB2 catalytic domain, a CPA1 catalytic domain, and a CPA4 catalytic domain.

In some embodiments, the fusion construct comprises a structural arrangement from N-terminus to C-terminus as (first peptide signal)-(CPB2 activation peptide)-(CPN2). In some embodiments, the fusion construct comprises a structural arrangement from C-terminus to N-terminus as (first peptide signal)-(CPB2 activation peptide)-(CPN2). In some embodiments, the fusion construct comprises a structural arrangement from N-terminus to C-terminus as (mMBP)-(optional linker)-(TEV)-(optional linker)-(CPN1 core molecule)-(optional linker)-(HSA). In some embodiments, the fusion construct comprises a structural arrangement from N-terminus to C-terminus as (mMBP)-(optional linker)-(TEV)-(optional linker)-(CPN1 core molecule). In some embodiments, the fusion construct comprises a structural arrangement from N-terminus to C-terminus as (mMBP)-(optional linker)-(TEV)-(optional linker)-(core CPN1 molecule)-(optional linker)-(TEV)-(optional linker)-(HSA). The mMBP can be SEQ ID NO: 30, the optional linker can be SEQ ID NOS: 26, 32, 33, 34, 35, 36, 37, 38, 39, 41, 42, or 43. The core CPN1 molecule can be SEQ ID NOS: 1, 6, 7, 11, 12, or 8. The TEV can be SEQ ID NO:25. The HSA can be SEQ ID NO: 17. In some embodiments, the fusion construct comprises a structural arrangement from N-terminus to C-terminus as (GST)-(optional linker)-(TEV)-CPN1 core molecule)-(optional linker)-(HSA). In some embodiments, the fusion construct comprises a structural arrangement from N-terminus to C-terminus as (GST)-(optional linker)-(TEV)-(optional linker)-(CPN core molecule)-(optional linker)-(TEV)-(optional linker)-(HSA). In some embodiments, the fusion construct comprises a structural arrangement from N-terminus to C-terminus as (GST)-(optional linker)-(TEV)-(core CPN1 molecule). The GST can be SEQ ID NO: 31. In some embodiments, the fusion construct comprises a structural arrangement from N-terminus to C-terminus as (GHHHHHHHHHH)-(optional linker)-(SUMO)-(CPN1 core molecule). In some embodiments, the fusion construct comprises a structural arrangement from N-terminus to C-terminus as (GHHHHHHHHHH)-(optional linker)-(SUMO)-(CPN1 core molecule)-(optional linker)-(HSA). The GHHHHHHHHHH can be SEQ ID NO: 34. The SUMO can be SEQ ID NO: 24. In some embodiments, the fusion construct comprises a structural arrangement from N-terminus to C-terminus as (CPB2-Activation Peptide)-(optional linker)-(CPN1 core molecule). The CPB2-Activation Peptide can be SEQ ID NO: 22 In some embodiments, the fusion construct comprises a structural arrangement from N-terminus to C-terminus as (CPB2-Activation Peptide)-(optional linker)-(CPN1 core molecule)-(optional linker)-(HHHHHH). The HHHHHH can be SEQ ID NO: 40. In some embodiments, the fusion construct comprises a structural arrangement from N-terminus to C-terminus as (CPB2-Activation Peptide)-(optional linker)-(CPN1 core molecule)-(optional linker)-(HSA). In some embodiments, the fusion construct comprises a structural arrangement from N-terminus to C-terminus as (CPB2-Activation Peptide)-(optional linker)-(CPN1 core molecule)-(optional linker)-(hCPN2 (1-339)). In some embodiments, the fusion construct comprises a structural arrangement from N-terminus to C-terminus as (CPB2-Activation Peptide)-(optional linker)-(CPN1 core molecule)-(optional linker)-(hCPN2 (1-339))-(optional linker)-(HHHHHH). In some embodiments, the fusion construct comprises a structural arrangement from N-terminus to C-terminus as (CPN1 core molecule)-(optional linker)-(hCPN2 (1-339)). In some embodiments, the fusion construct comprises a structural arrangement from N-terminus to C-terminus as (CPN1 core molecule)-(optional linker)-(hCPN2 (1-339))-(optional linker)-(HHHHHH). In some embodiments, the fusion construct comprises a structural arrangement from N-terminus to C-terminus as (CPN1 core molecule)-(optional linker)-(hCPN2 (1-456)). The hCPN2 (1-456) can be SEQ ID NO: 10. In some embodiments, the fusion construct comprises a structural arrangement from N-terminus to C-terminus as (CPN1 core molecule)-(optional linker)-(HHHHHH). In some embodiments, the fusion construct comprises a structural arrangement from N-terminus to C-terminus as (CPN1 core molecule)-(optional linker)-(HSA). In some embodiments, the fusion construct comprises a structural arrangement from N-terminus to C-terminus as (CPN1 core molecule)-(optional linker)-(HSA)-(optional linker)-(HHHHHH). In some embodiments, the fusion construct comprises a structural arrangement from N-terminus to C-terminus as (CPN1 core molecule)-(optional linker)-(TEV)-(optional linker)-(HSA). In some embodiments, the fusion construct comprises a structural arrangement from N-terminus to C-terminus as (CPN1 core molecule)-(optional linker)-(TEV)-(optional linker)-(Fc (no 1st DK LALA-PG)). The Fc (no 1st DK LALA-PG) can be SEQ ID NO: 28. In some embodiments, the fusion construct comprises a structural arrangement from N-terminus to C-terminus as (CPN1 core molecule)-(optional linker)-(Xa)-(optional linker)-(Fc (no 1st DK LALA-PG)). The Xa can be SEQ ID NO: 29. In some embodiments, the fusion construct comprises a structural arrangement from N-terminus to C-terminus as (CPN1 core molecule)-(optional linker)-(Xa)-(optional linker)-(HSA). In some embodiments, the fusion construct comprises a structural arrangement from N-terminus to C-terminus as (CPN1 core molecule)-(optional linker)-(HSA)-(optional linker)-(HHHHHHHHHH). In some embodiments, the fusion construct comprises a structural arrangement from N-terminus to C-terminus as CPN1 core molecule)-(optional linker)-(HSA).

Table 2 provides exemplary fusion constructs. The column titled “Const. No.” refers to the construct number, a unique number assigned to the construct. Each component of the fusion protein is presented from the N-terminus to the C-terminus in the subsequent columns, presented from N-terminus to C-terminus. If a cell is blank that indicates that the particular construct does not contain that component.

For example, the component in the left-most side of the table, if present, is found in the far N-terminus, labeled as “N2 Term.” This may then be connected via linker (“N2 Linker”) to another component in the N Terminus, if present, is closer to the core molecule, labeled “N Term.” The “N Term” component may then connected to the core molecule via an optional linker labeled “N Linker.” The column entitled “core” denotes the core CPN1 or CPN2 molecule, making reference to exemplary molecules in Table 1. To the right of the core molecule is an optional “C Linker”, which if present, links the core to a component in the “C Term,” which, in present, is in turn is connected to a “C2 Term” component via an optional “C2 Linker.”

In some embodiments, the fusion constructs provided herein are fused to a first non-CPN domain or component, and a second non-CPN domain or component. In some embodiments, the non-CPN domain or component is a half-life extender. In some embodiments, the half-life extender is selected from the group consisting of: PEGylation, PASylation, carbohydrates, albumin, and Fc. In some embodiments, the fusion is at the C-terminal end of the CPN1 core. In some embodiments, the fusion is at the N-terminal end of the fusion construct. In some embodiments, the fusion is at both the C-terminal and N-terminal ends of the CPN1 core. Other exemplary chimeras include fusions of CPN1 with: a portion of a redesigned CPN2, a portion of CPB2 activation peptide, or other CPB2 domains.

In some embodiments, the fusion of at least one non-CPN domain or component comprises a first non-CPN domain or component a second non-CPN domain or component. In some embodiments, the fusion construct comprises a structural arrangement from N-terminus to C-terminus as (first peptide signal)-(first non-CPN domain or component)-(CPN2)-(second non-CPN domain or component). In some embodiments, the fusion construct comprises a structural arrangement from C-terminus to N-terminus as (first peptide signal)-(first non-CPN domain or component)-(CPN2)-(second non-CPN domain or component). In some embodiments, the first non-CPN domain or component is an activation peptide. In some embodiments, the second non-CPN domain or component is a half-life extender. It may be advantageous in some embodiments to increase the half-life of CPN1. Exemplary half-life extenders include, but are not limited to albumin, such as human serum albumin, PEG, a non-biodegradable polymer, a biodegradable polymer, and Fc. Addition of a half-life extender can also increase or alter other properties of the fusion constructs provided herein, such as, but not limited to, bioavailability, trafficking ability, and immunogenicity.

In some embodiments, the half-life extender is albumin. It is noted that as used herein, albumin refers to any albumin such as any serum albumin, or an albumin variant, or albumin derivative. In exemplary embodiments, the albumin is human serum albumin (HSA). Exemplary albumin containing CPN1 fusion constructs of the disclosure are provided Table 2 and Table 5.

In some embodiments, the addition of a component or domain to a fusion construct of the disclosure can be directly to the CPN1 variant core molecule. In some embodiments, the addition of a component or domain to a fusion construct of the disclosure can be through a linker or multiple linkers.

As discussed above, CPN is endogenously found as a tetramer. Accordingly, in some embodiments, the CPN variants provided herein are a tetramer comprising two heterodimers. In some embodiments, the CPN variants provided herein is a single heterodimer.

In some embodiments, the CPN1 variants or fusion constructs provided herein are in a zymogen form. In such embodiments, the CPN1 variants or fusion constructs engineered as a zymogen can be activated in situ at the site of dysregulated complement. By way of example, a fusion construct can be provided fused an activation peptide, such as an activation peptide from CPB2, which can be cleaved at the site of dysregulated complement, such as by MASP1, MASP3, Factor D, mCPA3, or cathepsin G.

In some embodiments, the fusion constructs provided herein are in an active form.

### Uses

Provided herein are methods of treating a disease or condition in a subject in need thereof, comprising administering to the subject any one of the CPN1 variants or fusion constructs provided herein.

In some embodiments, the disease or condition is an acute condition. In some embodiments, the disease or condition is an acute condition, and the CPN1 variant or fusion construct of the disclosure is short-lived and highly active. Examples of acute conditions for which such fusion constructs and variants can be useful include, but are not limited to, acute respiratory distress syndrome (ARDS), COVID-19, multisystem organ failure, and sepsis. Additionally, such fusion constructs and variants can be useful for acute disease in which significant complement activation occurs during extracorporeal blood treatment by cardiopulmonary bypass surgery, ischemia/reperfusion, and dialysis. These can contribute to complications of these procedures.

In some embodiments, the disease or condition is a chronic condition. In some embodiments, the disease or condition is a chronic condition, and the fusion constructs or and variants have an extended half-life, and/or with a modified catalytic activity with respect to a wild type CPN1 or wild type CPN tetramer. Examples of chronic conditions for which such fusion constructs and variants can be useful include, but are not limited to, anti-neutrophil cytoplasmic autoantibody (ANCA) vasculitis, atypical hemolytic uremic syndrome (aHUS), and IgA nephropathy.

In some embodiments, the disease or condition is selected from the group consisting of: congenital complement deficiency, control protein deficiency, secondary complement disorder, immunity related disorder, chronic renal disorder, acute inflammatory disorder, antineutrophil cytoplasmic antibody (ANCA)-associated vasculitis (AAV), C3 glomerulopathy (C3G), lupus nephritis, skin disorder, intestinal ischemia and reperfusion (I/R) injury, and sepsis. In some embodiments, the disease or condition is a skin disorder selected from the group consisting of: hidradenitis suppurativa (HS), bullous pemphigoid (BP), and Pyoderma Gangrenosum.

### Administration

The in vivo administration of the CPN1 variants and fusion constructs described herein may be carried out intravenously, intramuscularly, subcutaneously, intravitreally, topically, orally, transdermally, intraperitoneally, intraorbitally, intrathecally, intraventricularly, intranasally, transmucosally, through implantation, or through inhalation. Administration may be performed with any suitable excipients, carriers, or other agents to provide suitable or improved tolerance, transfer, delivery, and the like.

In exemplary embodiments, administration of the therapeutic variants and fusion constructs described herein is a subcutaneous administration.

In exemplary embodiments, administration of the therapeutic variants and fusion constructs described herein is an intravenous administration.

Administration may be effectuated by delivery of the variant or fusion construct itself, or a gene therapy based delivery, using, for example, a viral vector.

### Production

Provided herein are nucleic acids encoding the CPN1 variants of the disclosure, and vector comprising such nucleic acids. Also provided herein are nucleic acids encoding the fusion constructs of the disclosure, and vector comprising such nucleic acids. These are useful in using recombinant systems for production. Mammalian host cells may be used for production, including, for example human and non-human cell production systems. Exemplary mammalian cells for production include, but are not limited to CHO cells, or HEK cells. Alternatively, non-mammalian systems may be used for recombinant production of the variants and fusion constructs of the disclosure, such as bacterial host systems (e.g. E. Coli), yeast, and insect cell systems. It is understood that the nucleic acids encoding the variants and fusion constructs of the disclosure may be codon optimized, to maximize production. The variants and fusion constructs provided herein can be cloned or isolated using any available methods known in the art for cloning and isolating nucleic acid molecules.

### TABLES

Provided here are the Tables made reference herein.

TABLE 1
Exemplary Components

| Name | Description | Amino Acid Sequence |
|---|---|---|
| hCPN1 (1- | Full length wild | MSDLLSVFLHLLLLFKLVAPVTFRHHRYDDLVRTLYKVQNECPGITRV |
| 438) with a | type human | YSIGRSVEGRHLYVLEFSDHPGIHEPLEPEVKYVGNMHGNEALGRELML |
| signal peptide | carboxypeptidase | QLSEFLCEEFRNRNQRIVQLIQDTRIHILPSMNPDGYEVAAAQGPNKPGY |
| | N 1, with a signal | LVGRNNANGVDLNRNFPDLNTYIYYNEKYGGPNHHLPLPDNWKSQVEP |
| | peptide | ETRAVIRWMHSFNFVLSANLHGGAVVANYPYDKSFEHRVRGVRRTAST |
| | | PTPDDKLFQKLAKVYSYAHGWMFQGWNCGDYFPDGITNGASWYSLSK |
| | | GMQDFNYLHTNCFEITLELSCDKFPPEEELQREWLGNREALIQFLEQVHQ |
| | | GIKGMVLDENYNNLANAVISVSGINHDVTSGDHGDYFRLLLPGIYTVSA |
| | | TAPGYDPETVTVTVGPAEPTLVNFHLKRSIPQVSPVRRAPSRRHGVRAKV |
| | | QPQARKKEMEMRQLQRGPA (SEQ ID NO: 1) |
| hCPN1 (1- | Full length wild | VTFRHHRYDDLVRTLYKVQNECPGITRVYSIGRSVEGRHLYVLEFSDHP |
| 438) | type human | GIHEPLEPEVKYVGNMHGNEALGRELMLQLSEFLCEEFRNRNQRIVQLIQ |
| | carboxypeptidase | DTRIHILPSMNPDGYEVAAAQGPNKPGYLVGRNNANGVDLNRNFPDLN |
| | N1 | TYIYYNEKYGGPNHHLPLPDNWKSQVEPETRAVIRWMHSFNFVLSANLH |
| | | GGAVVANYPYDKSFEHRVRGVRRTASTPTPDDKLFQKLAKVYSYAHG |
| | | WMFQGWNCGDYFPDGITNGASWYSLSKGMQDFNYLHTNCFEITLELSC |
| | | DKFPPEEELQREWLGNREALIQFLEQVHQGIKGMVLDENYNNLANAVIS |
| | | VSGINHDVTSGDHGDYFRLLLPGIYTVSATAPGYDPETVTVTVGPAEPTL |
| | | VNFHLKRSIPQVSPVRRAPSRRHGVRAKVQPQARKKEMEMRQLQRGPA |
| | | (SEQ ID NO: 6) |
| hCPN1 (1- | human | VTFRHHRYDDLVRTLYKVQNECPGITRVYSIGRSVEGRHLYVLEFSDHP |
| 398) | carboxypeptidase | GIHEPLEPEVKYVGNMHGNEALGRELMLQLSEFLCEEFRNRNQRIVQLIQ |
| | N 1, containing | DTRIHILPSMNPDGYEVAAAQGPNKPGYLVGRNNANGVDLNRNFPDLN |
| | only the N- | TYIYYNEKYGGPNHHLPLPDNWKSQVEPETRAVIRWMHSFNFVLSANLH |
| | terminal amino | GGAVVANYPYDKSFEHRVRGVRRTASTPTPDDKLFQKLAKVYSYAHG |
| | acids from 1-398 | WMFQGWNCGDYFPDGITNGASWYSLSKGMQDFNYLHTNCFEITLELSC |
| | | DKFPPEEELQREWLGNREALIQFLEQVHQGIKGMVLDENYNNLANAVIS |
| | | VSGINHDVTSGDHGDYFRLLLPGIYTVSATAPGYDPETVTVTVGPAEPTL |
| | | VNFHLKRS (SEQ ID NO: 7) |
| hCPN1 (1- | human | VTFRHHRYDDLVRTLYKVQNECPGITRVYSIGRSVEGRHLYVLEFSDHP |
| 397) | carboxypeptidase | GIHEPLEPEVKYVGNMHGNEALGRELMLQLSEFLCEEFRNRNQRIVQLIQ |
| | N 1, containing | DTRIHILPSMNPDGYEVAAAQGPNKPGYLVGRNNANGVDLNRNFPDLN |
| | only the N- | TYIYYNEKYGGPNHHLPLPDNWKSQVEPETRAVIRWMHSFNFVLSANLH |
| | terminal amino | GGAVVANYPYDKSFEHRVRGVRRTASTPTPDDKLFQKLAKVYSYAHG |
| | acids from 1-397 | WMFQGWNCGDYFPDGITNGASWYSLSKGMQDFNYLHTNCFEITLELSC |
| | | DKFPPEEELQREWLGNREALIQFLEQVHQGIKGMVLDENYNNLANAVIS |
| | | VSGINHDVTSGDHGDYFRLLLPGIYTVSATAPGYDPETVTVTVGPAEPTL |
| | | VNFHLKR (SEQ ID NO: 11) |
| hCPN1 (1- | human | VTFRHHRYDDLVRTLYKVQNECPGITRVYSIGRSVEGRHLYVLEFSDHP |
| 396) | carboxypeptidase | GIHEPLEPEVKYVGNMHGNEALGRELMLQLSEFLCEEFRNRNQRIVQLIQ |
| | N 1, containing | DTRIHILPSMNPDGYEVAAAQGPNKPGYLVGRNNANGVDLNRNFPDLN |
| | only the N- | TYIYYNEKYGGPNHHLPLPDNWKSQVEPETRAVIRWMHSFNFVLSANLH |
| | terminal amino | GGAVVANYPYDKSFEHRVRGVRRTASTPTPDDKLFQKLAKVYSYAHG |
| | acids from 1-396 | WMFQGWNCGDYFPDGITNGASWYSLSKGMQDFNYLHTNCFEITLELSC |
| | | DKFPPEEELQREWLGNREALIQFLEQVHQGIKGMVLDENYNNLANAVIS |
| | | VSGINHDVTSGDHGDYFRLLLPGIYTVSATAPGYDPETVTVTVGPAEPTL |
| | | VNFHLK (SEQ ID NO: 12) |
| hCPN1 (1- | human | VTFRHHRYDDLVRTLYKVQNECPGITRVYSIGRSVEGRHLYVLEFSDHP |
| 320 | carboxypeptidase | GIHEPLEPEVKYVGNMHGNEALGRELMLQLSEFLCEEFRNRNQRIVQLIQ |
| | N 1 C-terminal | DTRIHILPSMNPDGYEVAAAQGPNKPGYLVGRNNANGVDLNRNFPDLN |
| | truncation, | TYIYYNEKYGGPNHHLPLPDNWKSQVEPETRAVIRWMHSFNFVLSANLH |
| | containing only | GGAVVANYPYDKSFEHRVRGVRRTASTPTPDDKLFQKLAKVYSYAHG |
| | the N-terminal | WMFQGWNCGDYFPDGITNGASWYSLSKGMQDFNYLHTNCFEITLELSC |
| | amino acids from | DKFPPEEELQREWLGNREALIQFLEQVHQ (SEQ ID NO: 8) |
| | 1 to 320, deletion | |
| | of the transthyretin | |
| | domain | |
| hCPN2 (1- | Full length wild | MGWSLILLFLVAVATRVHSCPMGCDCFVQEVFCSDEELATVPLDIPPYT |
| 524) with | type human | KNIIFVETSFTTLETRAFGSNPNLTKVVFLNTQLCQFRPDAFGGLPRLEDL |
| signal peptide | carboxypeptidase | EVTGSSFLNLSTNIFSNLTSLGKLTLNFNMLEALPEGLFQHLAALESLHLQ |
| | N 2, with a signal | GNQLQALPRRLFQPLTHLKTLNLAQNLLAQLPEELFHPLTSLQTLKLSNN |
| | peptide | ALSGLPQGVFGKLGSLQELFLDSNNISELPPQVFSQLFCLERLWLQRNAIT |
| | | HLPLSIFASLGNLTFLSLQWNMLRVLPAGLFAHTPCLVGLSLTHNQLETV |
| | | AEGTFAHLSNLRSLMLSYNAITHLPAGIFRDLEELVKLYLGSNNLTALHP |
| | | ALFQNLSKLELLSLSKNQLTTLPEGIFDTNYNLFNLALHGNPWQCDCHLA |
| | | YLFNWLQQYTDRLLNIQTYCAGPAYLKGQVVPALNEKQLVCPVTRDHL |
| | | GFQVTWPDESKAGGSWDLAVQERAARSQCTYSNPEGTVVLACDQAQC |
| | | RWLNVQLSPQQGSLGLQYNASQEWDLRSSCGSLRLTVSIEARAAGP |
| | | (SEQ ID NO: 15) |
| hCPN2 (1- | Full length wild | CPMGCDCFVQEVFCSDEELATVPLDIPPYTKNIIFVETSFTTLETRAFGSNP |
| 524) | type human | NLTKVVFLNTQLCQFRPDAFGGLPRLEDLEVTGSSFLNLSTNIFSNLTSLG |
| | carboxypeptidase | KLTLNFNMLEALPEGLFQHLAALESLHLQGNQLQALPRRLFQPLTHLKT |
| | N 2 | LNLAQNLLAQLPEELFHPLTSLQTLKLSNNALSGLPQGVFGKLGSLQELF |
| | | LDSNNISELPPQVFSQLFCLERLWLQRNAITHLPLSIFASLGNLTFLSLQW |
| | | NMLRVLPAGLFAHTPCLVGLSLTHNQLETVAEGTFAHLSNLRSLMLSYN |
| | | AITHLPAGIFRDLEELVKLYLGSNNLTALHPALFQNLSKLELLSLSKNQLT |
| | | TLPEGIFDTNYNLFNLALHGNPWQCDCHLAYLFNWLQQYTDRLLNIQTY |
| | | CAGPAYLKGQVVPALNEKQLVCPVTRDHLGFQVTWPDESKAGGSWDL |
| | | AVQERAARSQCTYSNPEGTVVLACDQAQCRWLNVQLSPQQGSLGLQYN |
| | | ASQEWDLRSSCGSLRLTVSIEARAAGP (SEQ ID NO: 13) |
| hCPN2 (1- | wild type human | MGWSLILLFLVAVATRVHSCPMGCDCFVQEVFCSDEELATVPLDIPPYTK |
| 456) | carboxypeptidase | NIIFVETSFTTLETRAFGSNPNLTKVVFLNTQLCQFRPDAFGGLPRLEDLE |
| | N 2 containing | VTGSSFLNLSTNIFSNLTSLGKLTLNFNMLEALPEGLFQHLAALESLHLQG |
| | only the N- | NQLQALPRRLFQPLTHLKTLNLAQNLLAQLPEELFHPLTSLQTLKLSNNA |
| | terminal amino | LSGLPQGVFGKLGSLQELFLDSNNISELPPQVFSQLFCLERLWLQRNAITH |
| | acids from 1-456 | LPLSIFASLGNLTFLSLQWNMLRVLPAGLFAHTPCLVGLSLTHNQLETVA |
| | | EGTFAHLSNLRSLMLSYNAITHLPAGIFRDLEELVKLYLGSNNLTALHPA |
| | | LFQNLSKLELLSLSKNQLTTLPEGIFDTNYNLFNLALHGNPWQCDCHLAY |
| | | LFNWLQQYTDRLLNIQTYCAGPAYLKGQVVPALNEKQLVCPVTRDHLG |
| | | FQVTWPDESKAGGSWDLAVQERAA (SEQ ID NO: 10) |
| CPB2 with | Wild-type human | MKLCSLAVLVPIVLFCEQHVFAFQSGQVLAALPRTSRQVQVLQNLTTT |
| signal peptide | CPB2 (with signal | YEIVLWQPVTADLIVKKKQVHFFVNASDVDNVKAHLNVSGIPCSVLLAD |
| | peptide) | VEDLIQQQISNDTVSPR/ASASYYEQYHSLNEIYSWIEFITERHPDMLTKIH |
| | | IGSSFEKYPLYVLKVSGKEQAAKNAIWIDCGIHAREWISPAFCLWFIGHIT |
| | | QFYGIIGQYTNLLRLVDFYVMPVVNVDGYDYSWKKNRMWRKNRSFYA |
| | | NNHCIGTDLNRNFASKHWCEEGASSSSCSETYCGLYPESEPEVKAVASFL |
| | | RRNINQIKAYISMHSYSQHIVFPYSYTRSKSKDHEELSLVASEAVRAIEKIS |
| | | KNTRYTHGHGSETLYLAPGGGDDWIYDLGIKYSFTIELRDTGTYGFLLPE |
| | | RYIKPTCREAFAAVSKIAWHVIRNV (SEQ ID NO: 2) |
| | Exemplary CPN1 | MKLCSLAVLVPIVLFCEQHVFAFQSGQVLAALPRTSRQVQVLQNLTTTY |
| | construct | EIVLWQPVTADLIVKKKQVHFFVNASDVDNVKAHLNVSGIPCSVLLADV |
| | comprising a | EDLIQQQISNDTVSPR/ASASYYVTFRHHRYDDLVRTLYKVQNECPGITR |
| | CPB2 sequence | VYSIGRSVEGRHLYVLEFSDHPGIHEPLEPEVKYVGNMHGNEALGRELM |
| | | LQLSEFLCEEFRNRNQRIVQLIQDTRIHILPSMNPDGYEVAAAQGPNKPG |
| | | YLVGRNNANGVDLNRNFPDLNTYIYYNEKYGGPNHHLPLPDNWKSQVE |
| | | PETRAVIRWMHSFNFVLSANLHGGAVVANYPYDKSFEHRVRGVR/RTAS |
| | | TPTPDDKLFQKLAKVYSYAHGWMFQGWNCGDYFPDGITNGASWYSLS |
| | | KGMQDFNYLHTNCFEITLELSCDKFPPEEELQREWLGNREALIQFLEQVH |
| | | QGIKGMVLDENYNNLANAVIS (SEQ ID NO: 3) |
| TT | transthyretin | GIKGMVLDENYNNLANAVISVSGINHDVTSGDHGDYFRLLLPGIYTVSA |
| | domain | TAPGYDPETVTVTVGPAEPTLVNFHLKRS (SEQ ID NO: 16) |
| HSA | Human serum | DAHKSEVAHRFKDLGEENFKALVLIAFAQYLQQCPFEDHVKLVNEVTEF |
| | albumin | AKTCVADESAENCDKSLHTLFGDKLCTVATLRETYGEMADCCAKQEPE |
| | | RNECFLQHKDDNPNLPRLVRPEVDVMCTAFHDNEETFLKKYLYEIARRH |
| | | PYFYAPELLFFAKRYKAAFTECCQAADKAACLLPKLDELRDEGKASSAK |
| | | QRLKCASLQKFGERAFKAWAVARLSQRFPKAEFAEVSKLVTDLTKVHT |
| | | ECCHGDLLECADDRADLAKYICENQDSISSKLKECCEKPLLEKSHCIAEV |
| | | ENDEMPADLPSLAADFVESKDVCKNYAEAKDVFLGMFLYEYARRHPDY |
| | | SVVLLLRLAKTYETTLEKCCAAADPHECYAKVFDEFKPLVEEPQNLIKQ |
| | | NCELFEQLGEYKFQNALLVRYTKKVPQVSTPTLVEVSRNLGKVGSKCCK |
| | | HPEAKRMPCAEDYLSVVLNQLCVLHEKTPVSDRVTKCCTESLVNRRPCF |
| | | SALEVDETYVPKEFNAETFTFHADICTLSEKERQIKKQTALVELVKHKPK |
| | | ATKEQLKAVMDDFAAFVEKCCKADDKETCFAEEGKKLVAASQAALGL |
| | | (SEQ ID NO: 17) |
| signal HSA | signal sequence of | MKWVTFISLLFLFSSAYS (SEQ ID NO: 18) |
| | HSA | |
| signal | signal sequence of | MTRLTVLALLAGLLASSRA (SEQ ID NO: 19) |
| Azurocidin | Azurocidin | |
| signal IL2 | signal sequence of | MYRMQLLSCIALSLALVTNS (SEQ ID NO: 20) |
| | interleukin 2 | |
| signal Ig | signal sequence of | MGWSLILLFLVAVATRVHS (SEQ ID NO: 21) |
| | immunoglobin G | |
| Activation | Activation peptide | FQSGQVLAALPRTSRQVQVLQNLTTTYEIVLWQPVTADLIVKKKQVHFF |
| peptide | of CBP2, the N- | VNASDVDNVKAHLNVSGIPCSVLLADVEDLIQQQISNDTVSPRASAS |
| | terminal 96aa of | (SEQ ID NO: 22) |
| | CBP2 protease | |
| 10His-Sumo | 10 Histidine | GHHHHHHHHHHSLQDSEVNQEAKPEVKPEVKPETHINLKVSDGSSEIFF |
| | residues followed | KIKKTTPLRRLMEAFAKRQGKEMDSLTFLYDGIEIQADQTPEDLDMEDN |
| | by SUMO | DIIEAHREQIGG (SEQ ID NO: 23) |
| SUMO | Small Ubiquitin | SLQDSEVNQEAKPEVKPEVKPETHINLKVSDGSSEIFFKIKKTTPLRRLME |
| | Modifying | AFAKRQGKEMDSLTFLYDGIEIQADQTPEDLDMEDNDIIEAHREQIGG |
| | enzyme | (SEQ ID NO: 24) |
| TEV | Tobacco Etch | ENLYFQG (SEQ ID NO: 25) |
| | Virus protease | |
| | cleavage site | |
| LL | Long Linker | GSSGGSSGGSSGGSSGGSSG (SEQ ID NO: 26) |
| | (20amino acid- | |
| | length version of | |
| | GS linker) | |
| Fc | The fragment | THTCPPCPAPEAAGGPSVFLFPPKPKDTLMISRTPEVTCVVVDVSHEDPE |
| | crystallizable | VKFNWYVDGVEVHNAKTKPREEQYNSTYRVVSVLTVLHQDWLNGKEY |
| | region (Fc region) | KCKVSNKALGAPIEKTISKAKGQPREPQVYTLPPSRDELTKNQVSLTCLV |
| | is the tail region | KGFYPSDIAVEWESNGQPENNYKTTPPVLDSDGSFFLYSKLTVDKSRWQ |
| | of an antibody | QGNVFSCSVMHEALHNHYTQKSLSLSPGK (SEQ ID NO: 27) |
| | that interacts with | |
| | cell surface | |
| | receptors called | |
| | Fc receptors and | |
| | some proteins of | |
| | the complement | |
| | system. | |
| (no 1st DK | mutations | THTCPPCPAPEAAGGPSVFLFPPKPKDTLMISRTPEVTCVVVDVSHEDPE |
| LALA-PG) | introduced in the | VKFNWYVDGVEVHNAKTKPREEQYNSTYRVVSVLTVLHQDWLNGKEY |
| | Fc region | KCKVSNKALGAPIEKTISKAKGQPREPQVYTLPPSRDELTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK (SEQ ID NO: 28) |
| Xa | Factor Xa | AEGR (SEQ ID NO: 29) |
| | protease cleavage | |
| | site | |
| mMBP | mammalian | KTEEGKLVIWINGDKGYNGLAEVGKKFEKDTGIKVTVEHPDKLEEKFPQ |
| | maltose binding | VAATGDGPDIIFWAHDRFGGYAQSGLLAEITPAAAFQDKLYPFTWDAVR |
| | protein | YNGKLIAYPIAVEALSLIYNKDLLPNPPKTWEEIPALDKELKAKGKSALM |
| | | FNLQEPYFTWPLIAADGGYAFKYAAGKYDIKDVGVDNAGAKAGLTFLV |
| | | DLIKNKHMNADTDYSIAEHAFNHGETAMTINGPWAWSNIDTSAVNYGV |
| | | TVLPTFKGQPSKPFVGVLSAGINAASPNKELAKEFLENYLLTDEGLEAVN |
| | | KDKPLGAVALKSYEEELVKDPRVAATMENAQKGEIMPNIPQMSAFWYA |
| | | VRTAVINAASGRQTVDAAL (SEQ ID NO: 30) |
| GST | Glutathione S | MSPILGYWKIKGLVQPTRLLLEYLEEKYEEHLYERDEGDKWRNKKFELG |
| | transferase | LEFPNLPYYIDGDVKLTQSMAIIRYIADKHNMLGGCPKERAEISMLEGAV |
| | | LDIRYGVSRIAYSKDFETLKVDFLSKLPEMLKMFEDRLCHKTYLNGDHV |
| | | THPDFMLYDALDVVLYMDPMCLDAFPKLVCFKKRIEAIPQIDKYLKSSK |
| | | YIAWPLQGWQATFGGGDHPPK (SEQ ID NO: 31) |

TABLE 2
Exemplary Fusion Constructs

| Const. | N2 | N2 | N | NLink- | | | | C2Link- | |
|---|---|---|---|---|---|---|---|---|---|
| No. | Term | Linker | Term | er | Core | CLinker | CTerm | er | C2Term |
| 1 | | | | | hCPN1 | | | | |
| | | | | | (1-398) | | | | |
| 2 | | | | | hCPN1 | | | | |
| | | | | | (1-398) | | | | |
| 3 | | | | | hCPN1 | | | | |
| | | | | | (1-398) | | | | |
| 4 | | | | | hCPN1 | | | | |
| | | | | | (1-398) | | | | |
| 5 | | | | | hCPN1 | | HHHHHH | | |
| | | | | | (1-398) | | (SEQ | | |
| | | | | | | | ID | | |
| | | | | | | | NO: | | |
| | | | | | | | 32) | | |
| 6 | | | | | hCPN1 | | | GSSGG | HSA |
| | | | | | (1-398) | | | SSGGS | |
| | | | | | | | | SG | |
| | | | | | | | | (SEQ | |
| | | | | | | | | ID | |
| | | | | | | | | NO: | |
| | | | | | | | | 33) | |
| 7 | | | | | hCPN1 | GSSG | hCPN2 | | |
| | | | | | (1-398) | GSSG | (1-339) | | |
| | | | | | | GSSG | | | |
| | | | | | | (SEQ | | | |
| | | | | | | ID | | | |
| | | | | | | NO: | | | |
| | | | | | | 33) | | | |
| 8 | | | | | hCPN1 | GSSG | hCPN2 | | HHHH |
| | | | | | (1-398) | GSSG | (1-339) | | HH |
| | | | | | | GSSG | | | (SEQ |
| | | | | | | (SEQ | | | ID |
| | | | | | | ID | | | NO: |
| | | | | | | NO: | | | 32) |
| | | | | | | 33) | | | |
| 9 | | | CPB2- | | hCPN1 | | | | |
| | | | Activa- | | (1-398) | | | | |
| | | | tion | | | | | | |
| | | | peptide | | | | | | |
| 10 | | | CPB2- | | hCPN1 | | HHHH | | |
| | | | Activa- | | (1-398) | | HH | | |
| | | | tion | | | | (SEQ | | |
| | | | peptide | | | | ID | | |
| | | | | | | | NO: | | |
| | | | | | | | 32) | | |
| 11 | | | CPB2- | | hCPN1 | GSSG | HSA | | |
| | | | Activa- | | (1-398) | GSSG | | | |
| | | | tion | | | GSSG | | | |
| | | | peptide | | | (SEQ | | | |
| | | | | | | ID | | | |
| | | | | | | NO: | | | |
| | | | | | | 33) | | | |
| 12 | | | CPB2- | | hCPN1 | GSSG | hCPN2 | | |
| | | | Activa- | | (1-398) | GSSG | (1-339) | | |
| | | | tion | | | GSSG | | | |
| | | | peptide | | | (SEQ | | | |
| | | | | | | ID | | | |
| | | | | | | NO: | | | |
| | | | | | | 33) | | | |
| 13 | | | CPB2- | | hCPN1 | GSSG | hCPN2 | | HHHH |
| | | | Activa- | | (1-398) | GSSG | (1-339) | | HH |
| | | | tion | | | GSSG | | | (SEQ |
| | | | peptide | | | (SEQ | | | ID |
| | | | | | | ID | | | NO: |
| | | | | | | NO: | | | 32) |
| | | | | | | 33) | | | |
| 14 | GHHH | G | SUMO | | hCPN1 | | | | |
| | HHHH | | | | (1-398) | | | | |
| | HHH | | | | | | | | |
| | (SEQ | | | | | | | | |
| | ID | | | | | | | | |
| | NO: | | | | | | | | |
| | 34) | | | | | | | | |
| 15 | GHHH | G | SUMO | | hCPN1 | GSSG | HSA | | |
| | HHHH | | | | (1-398) | GSSG | | | |
| | HHH | | | | | GSSG | | | |
| | (SEQ | | | | | (SEQ | | | |
| | ID | | | | | ID | | | |
| | NO: | | | | | NO: | | | |
| | 34) | | | | | 33) | | | |
| 16 | GHHH | G | SUMO | | hCPN1 | | | | |
| | HHHH | | | | (1-320); | | | | |
| | HHH | | | | A | | | | |
| | (SEQ | | | | (321-398) | | | | |
| | ID | | | | | | | | |
| | NO: | | | | | | | | |
| | 34) | | | | | | | | |
| 17 | GHHH | G | SUMO | | hCPN1 | GSSG | HSA | | |
| | HHHH | | | | (1-320); | GSSG | | | |
| | HHH | | | | A | GSSG | | | |
| | (SEQ | | | | (321-398) | (SEQ | | | |
| | ID | | | | | ID | | | |
| | NO: | | | | | NO: | | | |
| | 34) | | | | | 33) | | | |
| 18 | | | | | hCPN1 | | | | |
| | | | | | (1-320); | | | | |
| | | | | | A | | | | |
| | | | | | (321-398) | | | | |
| 19 | | | | | hCPN1 | GSSG | HSA | | |
| | | | | | (1-320); | GSSG | | | |
| | | | | | A | GSSG | | | |
| | | | | | (321-398) | (SEQ | | | |
| | | | | | | ID | | | |
| | | | | | | NO: | | | |
| | | | | | | 33) | | | |
| 20 | | | | | hCPN1 | | TEV | GSSG | HSA |
| | | | | | (1-398) | | | GSSG | |
| | | | | | | | | GSSG | |
| | | | | | | | | (SEQ | |
| | | | | | | | | ID | |
| | | | | | | | | NO: | |
| | | | | | | | | 33) | |
| 21 | | | | | hCPN1 | | TEV | GSSG | HSA |
| | | | | | (1-398) | | | GSSG | |
| | | | | | | | | GSSG | |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 33) | |
| 22 | | | | | hCPN1 | | TEV | GSSG | Fc |
| | | | | | (1-398) | | | GSSG | (no 1st |
| | | | | | | | | GSSG | DKLA |
| | | | | | | | | (SEQ | LA-PG) |
| | | | | | | | | ID | |
| | | | | | | | | NO: | |
| | | | | | | | | 33) | |
| 23 | | | | | hCPN1 | | Xa | GSSG | Fc |
| | | | | | (1-398); | | | GSSG | (no 1st |
| | | | | | R37G | | | GSSG | DKLA |
| | | | | | | | | (SEQ | LA-PG) |
| | | | | | | | | ID | |
| | | | | | | | | NO: | |
| | | | | | | | | 33) | |
| 24 | | | | | hCPN1 | | Xa | GSSG | HSA |
| | | | | | (1-398); | | | GSSG | |
| | | | | | R37G | | | GSSG | |
| | | | | | | | | (SEQ | |
| | | | | | | | | ID | |
| | | | | | | | | NO: | |
| | | | | | | | | 33) | |
| 25 | mMBP | AAAQ | TEV | | hCPN1 | GSSG | HSA | | |
| | | TNAA | | | (1-398) | GSSG | | | |
| | | AASS | | | | GSSG | | | |
| | | ASLE | | | | (SEQ | | | |
| | | (SEQ | | | | ID | | | |
| | | ID | | | | NO: | | | |
| | | NO: | | | | 33) | | | |
| | | 35) | | | | | | | |
| 26 | mMBP | AAAQ | TEV | | hCPN1 | | | | |
| | | TNAA | | | (1-398) | | | | |
| | | AASS | | | | | | | |
| | | ASLE | | | | | | | |
| | | (SEQ | | | | | | | |
| | | ID | | | | | | | |
| | | NO: | | | | | | | |
| | | 35) | | | | | | | |
| 27 | GST | AAAQ | TEV | | hCPN1 | GSSG | HSA | | |
| | | TNAA | | | (1-398) | GSSG | | | |
| | | AASS | | | | GSSG | | | |
| | | ASLE | | | | (SEQ | | | |
| | | (SEQ | | | | ID | | | |
| | | ID | | | | NO: | | | |
| | | NO: | | | | 33) | | | |
| | | 35) | | | | | | | |
| 28 | | | | | hCPN1 | GSSG | hCPN2 | | |
| | | | | | (1-398) | GSSG | (1-456) | | |
| | | | | | | GSSG | | | |
| | | | | | | GSSG | | | |
| | | | | | | GSSG | | | |
| | | | | | | (SEQ | | | |
| | | | | | | ID | | | |
| | | | | | | NO: | | | |
| | | | | | | 26) | | | |
| 29 | | | | | hCPN2 | | | | |
| | | | | | (1-456) | | | | |
| 30 | | | | | hCPN2 | | | | |
| | | | | | (1-524) | | | | |
| 31 | | | | | hCPN1 | | | | |
| | | | | | (1-320); | | | | |
| | | | | | L55N; | | | | |
| | | | | | H161R; | | | | |
| | | | | | N188D; | | | | |
| | | | | | V190K; | | | | |
| | | | | | K229E; | | | | |
| | | | | | L230W; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 32 | | | | | hCPN1 | | | | |
| | | | | | (1-320); | | | | |
| | | | | | L55N; | | | | |
| | | | | | H161S; | | | | |
| | | | | | N188D; | | | | |
| | | | | | V190R; | | | | |
| | | | | | K229E; | | | | |
| | | | | | L230W; | | | | |
| | | | | | C282S; | | | | |
| | | | | | F283Y; | | | | |
| | | | | | R308K; | | | | |
| | | | | | Q320N | | | | |
| 33 | | | | | hCPN1 | | | | |
| | | | | | (1-320); | | | | |
| | | | | | L55N; | | | | |
| | | | | | H161K; | | | | |
| | | | | | N188D; | | | | |
| | | | | | V190K; | | | | |
| | | | | | K229E; | | | | |
| | | | | | L230W; | | | | |
| | | | | | C282S; | | | | |
| | | | | | F283Y; | | | | |
| | | | | | R308K; | | | | |
| | | | | | Q320N | | | | |
| 34 | | | | | hCPN1 | | | | |
| | | | | | (1-320); | | | | |
| | | | | | L55N; | | | | |
| | | | | | H161S; | | | | |
| | | | | | N188D; | | | | |
| | | | | | V190R; | | | | |
| | | | | | K229E; | | | | |
| | | | | | L230K; | | | | |
| | | | | | K233D; | | | | |
| | | | | | C282S; | | | | |
| | | | | | F283Y; | | | | |
| | | | | | R308K; | | | | |
| | | | | | Q320K | | | | |
| 35 | | | | | hCPN1 | | | | |
| | | | | | (1-320); | | | | |
| | | | | | L55N; | | | | |
| | | | | | H161R; | | | | |
| | | | | | N188D; | | | | |
| | | | | | V190K; | | | | |
| | | | | | K229E; | | | | |
| | | | | | L230W; | | | | |
| | | | | | K233H; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 36 | | | | | hCPN1 | | | | |
| | | | | | (1-320); | | | | |
| | | | | | L55N; | | | | |
| | | | | | H161R; | | | | |
| | | | | | N188D; | | | | |
| | | | | | V190K; | | | | |
| | | | | | K229E; | | | | |
| | | | | | L230K; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 37 | | | | | hCPN1 | | | | |
| | | | | | (1-438) | | | | |
| 38 | | | | | hCPN1 | GSSG | HSA | | |
| | | | | | (1-438) | GSSG | | | |
| | | | | | | GSSG | | | |
| | | | | | | (SEQ | | | |
| | | | | | | ID | | | |
| | | | | | | NO: | | | |
| | | | | | | 33) | | | |
| 39 | | | | | hCPN1 | GSSG | CPN2 | | |
| | | | | | (1-438) | GSSG | (1-456) | | |
| | | | | | | GSSG | | | |
| | | | | | | GSSG | | | |
| | | | | | | GSSG | | | |
| | | | | | | (SEQ | | | |
| | | | | | | ID | | | |
| | | | | | | NO: | | | |
| | | | | | | 26) | | | |
| 40 | | | | | hCPN1 | GSSG | CPN2 | | |
| | | | | | (1-438) | GSSG | (1-524) | | |
| | | | | | | GSSG | | | |
| | | | | | | GSSG | | | |
| | | | | | | GSSG | | | |
| | | | | | | (SEQ | | | |
| | | | | | | ID | | | |
| | | | | | | NO: | | | |
| | | | | | | 26) | | | |
| 41 | | | | | hCPN1 | GGSS | CPN2 | | |
| | | | | | (1-397) | GGSS | (1-425) | | |
| | | | | | | GGSS | | | |
| | | | | | | GGSS | | | |
| | | | | | | GGSS | | | |
| | | | | | | GGSS | | | |
| | | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 42 | | | | | hCPN1 | GGSS | CPN2 | | CD180 |
| | | | | | (1-397) | GGSS | (1-370) | | (45 |
| | | | | | | GGSS | | | residue |
| | | | | | | GGSS | | | capping |
| | | | | | | GGSS | | | motif) |
| | | | | | | GGSS | | | |
| | | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 43 | | | | | hCPN1 | GGSS | CPN2 | | LRIG1 |
| | | | | | (1-397) | GGSS | (1-367) | | (59 |
| | | | | | | GGSS | | | residue |
| | | | | | | GGSS | | | capping |
| | | | | | | GGSS | | | motif) |
| | | | | | | GGSS | | | |
| | | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 44 | | | | | hCPN1 | GGSS | CPN2 | | |
| | | | | | (1-396) | GGSS | (1-425) | | |
| | | | | | | GGSS | | | |
| | | | | | | GGSS | | | |
| | | | | | | GGSS | | | |
| | | | | | | GGSS | | | |
| | | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 45 | | | | | hCPN1 | GGSS | CPN2 | | CD180 |
| | | | | | (1-396) | GGSS | (1-425) | | (45 |
| | | | | | | GGSS | | | residue |
| | | | | | | GGSS | | | capping |
| | | | | | | GGSS | | | motif) |
| | | | | | | GGSS | | | |
| | | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 46 | | | | | hCPN1 | GGSS | CPN2 | | LRIG1 |
| | | | | | (1-396) | GGSS | (1-425) | | (59 |
| | | | | | | GGSS | | | residue |
| | | | | | | GGSS | | | capping |
| | | | | | | GGSS | | | motif) |
| | | | | | | GGSS | | | |
| | | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 47 | | | | | hCPN1 | GGSS | CPN2 | | |
| | | | | | (1-397) | GGSS | (1-425) | | |
| | | | | | | GGSS | | | |
| | | | | | | GGSS | | | |
| | | | | | | GGSS | | | |
| | | | | | | GGSS | | | |
| | | | | | | GG | | | |
| | | | | | | (SEQ | | | |
| | | | | | | ID | | | |
| | | | | | | NO: | | | |
| | | | | | | 37) | | | |
| 48 | | | | | hCPN1 | GGSS | CPN2 | | |
| | | | | | (1-397) | GGSS | (1-425) | | |
| | | | | | | GGSS | | | |
| | | | | | | GGSS | | | |
| | | | | | | GG | | | |
| | | | | | | SSGG | | | |
| | | | | | | SSGG | | | |
| | | | | | | SSGG | | | |
| | | | | | | SSGG | | | |
| | | | | | | SSSS | | | |
| | | | | | | GG | | | |
| | | | | | | (SEQ | | | |
| | | | | | | ID | | | |
| | | | | | | NO: | | | |
| | | | | | | 38) | | | |
| 49 | | | | | hCPN1 | GGSS | CPN2 | | CD180 |
| | | | | | (1-396) | GGSS | (1-370) | | (45 |
| | | | | | | GGSS | | | residue |
| | | | | | | GGSS | | | capping |
| | | | | | | GGSS | | | motif) |
| | | | | | | GGSS | | | |
| | | | | | | GG | | | |
| | | | | | | (SEQ | | | |
| | | | | | | ID | | | |
| | | | | | | NO: | | | |
| | | | | | | 37) | | | |
| 50 | | | | | hCPN1 | GGSS | CPN2 | | CD180 |
| | | | | | (1-396) | GGSS | (1-370) | | (45 |
| | | | | | | GGSS | | | residue |
| | | | | | | GGSS | | | capping |
| | | | | | | GGSS | | | motif) |
| | | | | | | GGSS | | | |
| | | | | | | GGSS | | | |
| | | | | | | GGSS | | | |
| | | | | | | GGSS | | | |
| | | | | | | SSGG | | | |
| | | | | | | (SEQ | | | |
| | | | | | | ID | | | |
| | | | | | | NO: | | | |
| | | | | | | 38) | | | |
| 51 | GHHH | G | SUMO | | hCPN1 | GGSS | CPN2 | | |
| | HHHH | | | | (1-397) | GGSS | (1-425) | | |
| | HHH | | | | | GGSS | | | |
| | (SEQ | | | | | GGSS | | | |
| | ID | | | | | GGSS | | | |
| | NO: | | | | | GGSS | | | |
| | 34) | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 52 | GHHH | G | SUMO | | hCPN1 | GGSS | CPN2 | | CD180 |
| | HHHH | | | | (1-397) | GGSS | (1-370) | | (45 |
| | HHH | | | | | GGSS | | | residue |
| | (SEQ | | | | | GGSS | | | capping |
| | ID | | | | | GGSS | | | motif) |
| | NO: | | | | | GGSS | | | |
| | 34) | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 53 | GHHH | G | SUMO | | hCPN1 | GGSS | CPN2 | | LRIG1 |
| | HHHH | | | | (1-397) | GGSS | (1-367) | | (59 |
| | HHH | | | | | GGSS | | | residue |
| | (SEQ | | | | | GGSS | | | capping |
| | ID | | | | | GGSS | | | motif) |
| | NO: | | | | | GGSS | | | |
| | 34) | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 54 | GHHH | G | SUMO | | hCPN1 | GGSS | CPN2 | | |
| | HHHH | | | | (1-396) | GGSS | (1-425) | | |
| | HHH | | | | | GGSS | | | |
| | (SEQ | | | | | GGSS | | | |
| | ID | | | | | GGSS | | | |
| | NO: | | | | | GGSS | | | |
| | 34) | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 55 | GHHH | G | SUMO | | hCPN1 | GGSS | CPN2 | | CD180 |
| | HHHH | | | | (1-396) | GGSS | (1-425) | | (45 |
| | HHH | | | | | GGSS | | | residue |
| | (SEQ | | | | | GGSS | | | capping |
| | ID | | | | | GGSS | | | motif) |
| | NO: | | | | | GGSS | | | |
| | 34) | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 56 | GHHH | G | SUMO | | hCPN1 | GGSS | CPN2 | | LRIG |
| | HHHH | | | | (1-396) | GGSS | (1-425) | | 1 |
| | HHH | | | | | GGSS | | | (59 |
| | (SEQ | | | | | GGSS | | | residue |
| | ID | | | | | GGSS | | | capping |
| | NO: | | | | | GGSS | | | motif) |
| | 34) | | | | | GGSS | | | |
| | | | | | | GGS | | | |
| | | | | | | (SEQ | | | |
| | | | | | | ID | | | |
| | | | | | | NO: | | | |
| | | | | | | 36) | | | |
| 57 | GHHH | G | SUMO | | hCPN1 | GGSS | CPN2 | | |
| | HHHH | | | | (1-397) | GGSS | (1-425) | | |
| | HHH | | | | | GGSS | | | |
| | (SEQ | | | | | GGSS | | | |
| | ID | | | | | GGSS | | | |
| | NO: | | | | | GGSS | | | |
| | 34) | | | | | GG | | | |
| | | | | | | (SEQ | | | |
| | | | | | | ID | | | |
| | | | | | | NO: | | | |
| | | | | | | 37) | | | |
| 58 | GHHH | G | SUMO | | hCPN1 | GGSS | CPN2 | | |
| | HHHH | | | | (1-397) | GGSS | (1-425) | | |
| | HHH | | | | | GGSS | | | |
| | (SEQ | | | | | GGSS | | | |
| | ID | | | | | GGSS | | | |
| | NO: | | | | | GGSS | | | |
| | 34) | | | | | GGSS | | | |
| | | | | | | GGSS | | | |
| | | | | | | GGSS | | | |
| | | | | | | SSGG | | | |
| | | | | | | (SEQ | | | |
| | | | | | | ID | | | |
| | | | | | | NO: | | | |
| | | | | | | 38) | | | |
| 59 | GHHH | G | SUMO | | hCPN1 | GGSS | CPN2 | | CD180 |
| | HHHH | | | | (1-396) | GGSS | (1-370) | | (45 |
| | HHH | | | | | GGSS | | | residue |
| | (SEQ | | | | | GGSS | | | capping |
| | ID | | | | | GGSS | | | motif) |
| | NO: | | | | | GGSS | | | |
| | 34) | | | | | GG | | | |
| | | | | | | (SEQ | | | |
| | | | | | | ID | | | |
| | | | | | | NO: | | | |
| | | | | | | 37) | | | |
| 60 | GHHH | G | SUMO | | hCPN1 | GGSS | CPN2 | | CD180 |
| | HHHH | | | | (1-396) | GGSS | (1-370) | | (45 |
| | HHH | | | | | GGSS | | | residue |
| | (SEQ | | | | | GGSS | | | capping |
| | ID | | | | | GGSS | | | motif) |
| | NO: | | | | | GGSS | | | |
| | 34) | | | | | GGSS | | | |
| | | | | | | GGSS | | | |
| | | | | | | GGSS | | | |
| | | | | | | SSGG | | | |
| | | | | | | (SEQ | | | |
| | | | | | | ID | | | |
| | | | | | | NO: | | | |
| | | | | | | 38) | | | |
| 61 | | | | | hCPN1 | | | GSSG | HSA |
| | | | | | (1-398); | | | GSSG | |
| | | | | | K60F; | | | GSSG | |
| | | | | | V62S; | | | (SEQ | |
| | | | | | V180Y; | | | ID | |
| | | | | | S192A; | | | NO: | |
| | | | | | N276T; | | | 33) | |
| | | | | | E284I | | | | |
| 62 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | N276V; | | | NO: | |
| | | | | | E284I | | | 33) | |
| 63 | | | | | hCPN1 | | | GSSG | HSA |
| | | | | | (1-398); | | | GSSG | |
| | | | | | K60F; | | | GSSG | |
| | | | | | V62M; | | | (SEQ | |
| | | | | | V180A; | | | ID | |
| | | | | | S192A; | | | NO: | |
| | | | | | M272F; | | | 33) | |
| | | | | | N276V; | | | | |
| | | | | | E284I | | | | |
| 64 | | | | | hCPN1 | | | GSSG | HSA |
| | | | | | (1-398); | | | GSSG | |
| | | | | | K60A; | | | GSSG | |
| | | | | | V62L; | | | (SEQ | |
| | | | | | M184L; | | | ID | |
| | | | | | S192M; | | | NO: | |
| | | | | | M272L; | | | 33) | |
| | | | | | N276I; | | | | |
| | | | | | E284V | | | | |
| 65 | | | | | hCPN1 | | | GSSG | HSA |
| | | | | | (1-398); | | | GSSG | |
| | | | | | A193W; | | | GSSG | |
| | | | | | A202V; | | | (SEQ | |
| | | | | | Y238F; | | | ID | |
| | | | | | I285L; | | | NO: | |
| | | | | | L287I; | | | 33) | |
| | | | | | L289Y; | | | | |
| | | | | | E314A | | | | |
| 66 | | | | | hCPN1 | | | GSSG | HSA |
| | | | | | (1-398); | | | GSSG | |
| | | | | | A202V; | | | GSSG | |
| | | | | | L287I; | | | (SEQ | |
| | | | | | L289T | | | ID | |
| | | | | | | | | NO: | |
| | | | | | | | | 33) | |
| 67 | | | | | hCPN1 | | | GSSG | HSA |
| | | | | | (1-398); | | | GSSG | |
| | | | | | A202I | | | GSSG | |
| | | | | | | | | (SEQ | |
| | | | | | | | | ID | |
| | | | | | | | | NO: | |
| | | | | | | | | 33) | |
| 68 | | | | | hCPN1 | | | GSSG | HSA |
| | | | | | (1-398); | | | GSSG | |
| | | | | | A235L | | | GSSG | |
| | | | | | | | | (SEQ | |
| | | | | | | | | ID | |
| | | | | | | | | NO: | |
| | | | | | | | | 33) | |
| 69 | | | | | hCPN1 | | | GSSG | HSA |
| | | | | | (1-398); | | | GSSG | |
| | | | | | V1I; | | | GSSG | |
| | | | | | F3W; | | | (SEQ | |
| | | | | | E74L; | | | ID | |
| | | | | | Q78W | | | NO: | |
| | | | | | | | | 33) | |
| 70 | | | | | hCPN1 | | | GSSG | HSA |
| | | | | | (1-398); | | | GSSG | |
| | | | | | V1I; | | | GSSG | |
| | | | | | E74L; | | | (SEQ | |
| | | | | | Q78W | | | ID | |
| | | | | | | | | NO: | |
| | | | | | | | | 33) | |
| 71 | | | | | hCPN1 | GSSG | HSA | | |
| | | | | | (1-398); | GSSG | | | |
| | | | | | K60F; | GSSG | | | |
| | | | | | V62S; | (SEQ | | | |
| | | | | | V180Y; | ID | | | |
| | | | | | S192A; | NO: | | | |
| | | | | | N276T; | 33) | | | |
| | | | | | E284I | | | | |
| 72 | | | | | hCPN1 | GSSG | HSA | | |
| | | | | | (1-398); | GSSG | | | |
| | | | | | K60F; | GSSG | | | |
| | | | | | V180F; | (SEQ | | | |
| | | | | | S192A; | ID | | | |
| | | | | | N276V; | NO: | | | |
| | | | | | E284I | 33) | | | |
| 73 | | | | | hCPN1 | GSSG | HSA | | |
| | | | | | (1-398); | GSSG | | | |
| | | | | | K60F; | GSSG | | | |
| | | | | | V62M; | (SEQ | | | |
| | | | | | V180A; | ID | | | |
| | | | | | S192A; | NO: | | | |
| | | | | | M272F; | 33) | | | |
| | | | | | N276V; | | | | |
| | | | | | E284I | | | | |
| 74 | | | | | hCPN1 | GSSG | HSA | | |
| | | | | | (1-398); | GSSG | | | |
| | | | | | K60A; | GSSG | | | |
| | | | | | V62L; | (SEQ | | | |
| | | | | | M184L; | ID | | | |
| | | | | | S192M; | NO: | | | |
| | | | | | M272L; | 33) | | | |
| | | | | | N2761; | | | | |
| | | | | | E284V | | | | |
| 75 | | | | | hCPN1 | GSSG | HSA | | |
| | | | | | (1-398); | GSSG | | | |
| | | | | | A193W; | GSSG | | | |
| | | | | | A202V; | (SEQ | | | |
| | | | | | Y238F; | ID | | | |
| | | | | | I285L; | NO: | | | |
| | | | | | L287I; | 33) | | | |
| | | | | | L289Y; | | | | |
| | | | | | E314A | | | | |
| 76 | | | | | hCPN1 | GSSG | HSA | | |
| | | | | | (1-398); | GSSG | | | |
| | | | | | A202V; | GSSG | | | |
| | | | | | L287I; | (SEQ | | | |
| | | | | | L289T | ID | | | |
| | | | | | | NO: | | | |
| | | | | | | 33) | | | |
| 77 | | | | | hCPN1 | GSSG | HSA | | |
| | | | | | (1-398); | GSSG | | | |
| | | | | | A202I | GSSG | | | |
| | | | | | | (SEQ | | | |
| | | | | | | ID | | | |
| | | | | | | NO: | | | |
| | | | | | | 33) | | | |
| 78 | | | | | hCPN1 | GSSG | HSA | | |
| | | | | | (1-398); | GSSG | | | |
| | | | | | A235L | GSSG | | | |
| | | | | | | (SEQ | | | |
| | | | | | | ID | | | |
| | | | | | | NO: | | | |
| | | | | | | 33) | | | |
| 79 | | | | | hCPN1 | GSSG | HSA | | |
| | | | | | (1-398); | GSSG | | | |
| | | | | | V1I; | GSSG | | | |
| | | | | | F3W; | (SEQ | | | |
| | | | | | E74L; | ID | | | |
| | | | | | Q78W | NO: | | | |
| | | | | | | 33) | | | |
| 80 | | | | | hCPN1 | GSSG | HSA | | |
| | | | | | (1-398); | GSSG | | | |
| | | | | | V1I; | GSSG | | | |
| | | | | | E74L; | (SEQ | | | |
| | | | | | Q78W | ID | | | |
| | | | | | | NO: | | | |
| | | | | | | 33) | | | |
| 81 | | | | | hCPN1 | GSSG | HSA | | |
| | | | | | (1-398); | GSSG | | | |
| | | | | | A70V | GSSG | | | |
| | | | | | | (SEQ | | | |
| | | | | | | ID | | | |
| | | | | | | NO: | | | |
| | | | | | | 33) | | | |
| 82 | | | | | hCPN1 | GSSG | HSA | | |
| | | | | | (1-398); | GSSG | | | |
| | | | | | N68D; | GSSG | | | |
| | | | | | P125S | (SEQ | | | |
| | | | | | | ID | | | |
| | | | | | | NO: | | | |
| | | | | | | 33) | | | |
| 83 | | | | | hCPN1 | GSSG | HSA | | |
| | | | | | (1-398); | GSSG | | | |
| | | | | | N68D; | GSSG | | | |
| | | | | | A70V; | (SEQ | | | |
| | | | | | P125S | ID | | | |
| | | | | | | NO: | | | |
| | | | | | | 33) | | | |
| 84 | | | | | hCPN1 | | TEV | GSSG | HSA |
| | | | | | (1-398); | | | GSSG | |
| | | | | | E288Q | | | GSSG | |
| | | | | | | | | (SEQ | |
| | | | | | | | | ID | |
| | | | | | | | | NO: | |
| | | | | | | | | 33) | |
| 85 | | | | | hCPN1 | | TEV | GSSG | HSA |
| | | | | | (1-398); | | | GSSG | |
| | | | | | Y266F | | | GSSG | |
| | | | | | | | | (SEQ | |
| | | | | | | | | ID | |
| | | | | | | | | NO: | |
| | | | | | | | | 33) | |
| 86 | | | | | hCPN1 | | TEV | GSSG | HSA |
| | | | | | (1-398); | | | GSSG | |
| | | | | | Y266F; | | | GSSG | |
| | | | | | E288Q | | | (SEQ | |
| | | | | | | | | ID | |
| | | | | | | | | NO: | |
| | | | | | | | | 33) | |
| 87 | | | | | hCPN1 | | TEV | GSSG | HSA |
| | | | | | (1-398); | | | GSSG | |
| | | | | | A199S | | | GSSG | |
| | | | | | | | | (SEQ | |
| | | | | | | | | ID | |
| | | | | | | | | NO: | |
| | | | | | | | | 33) | |
| 88 | | | | | hCPN1 | | TEV | GSSG | HSA |
| | | | | | (1-398); | | | GSSG | |
| | | | | | K124R; | | | GSSG | |
| | | | | | P125S | | | (SEQ | |
| | | | | | | | | ID | |
| | | | | | | | | NO: | |
| | | | | | | | | 33) | |
| 89 | | | | | hCPN1 | | TEV | GSSG | HSA |
| | | | | | (1-398); | | | GSSG | |
| | | | | | G121T; | | | GSSG | |
| | | | | | P122Y; | | | (SEQ | |
| | | | | | K124Q; | | | ID | |
| | | | | | P125D | | | NO: | |
| | | | | | | | | 33) | |
| 90 | | | | | hCPN1 | | TEV | GSSG | HSA |
| | | | | | (1-398); | | | GSSG | |
| | | | | | N123Y; | | | GSSG | |
| | | | | | K124R | | | (SEQ | |
| | | | | | | | | ID | |
| | | | | | | | | NO: | |
| | | | | | | | | 33) | |
| 91 | | | | | hCPN1 | | TEV | GSSG | HSA |
| | | | | | (1-398); | | | GSSG | |
| | | | | | G121D; | | | GSSG | |
| | | | | | P122D; | | | (SEQ | |
| | | | | | N123T; | | | ID | |
| | | | | | K124I | | | NO: | |
| | | | | | | | | 33) | |
| 92 | | | | | hCPN1 | | TEV | GSSG | HSA |
| | | | | | (1-398); | | | GSSG | |
| | | | | | G121D; | | | GSSG | |
| | | | | | P122H; | | | (SEQ | |
| | | | | | K124I | | | ID | |
| | | | | | | | | NO: | |
| | | | | | | | | 33) | |
| 93 | | | | | hCPN1 | | TEV | GSSG | HSA |
| | | | | | (1-398); | | | GSSG | |
| | | | | | G121R; | | | GSSG | |
| | | | | | P122E; | | | (SEQ | |
| | | | | | N123Y; | | | ID | |
| | | | | | K124I | | | NO: | |
| | | | | | | | | 33) | |
| 94 | | | | | hCPN1 | | TEV | GSSG | HSA |
| | | | | | (1-398); | | | GSSG | |
| | | | | | G121D; | | | GSSG | |
| | | | | | P122H; | | | (SEQ | |
| | | | | | K124Q | | | ID | |
| | | | | | | | | NO: | |
| | | | | | | | | 33) | |
| 95 | | | | | hCPN1 | | TEV | GSSG | HSA |
| | | | | | (1-398); | | | GSSG | |
| | | | | | G121I; | | | GSSG | |
| | | | | | N123D | | | (SEQ | |
| | | | | | | | | ID | |
| | | | | | | | | NO: | |
| | | | | | | | | 33) | |
| 96 | | | | | hCPN1 | | TEV | GSSG | HSA |
| | | | | | (1-398); | | | GSSG | |
| | | | | | P122W; | | | GSSG | |
| | | | | | N123R; | | | (SEQ | |
| | | | | | K124Y | | | ID | |
| | | | | | | | | NO: | |
| | | | | | | | | 33) | |
| 97 | | | | | hCPN1 | | TEV | GSSG | HSA |
| | | | | | (1-398); | | | GSSG | |
| | | | | | P122Y; | | | GSSG | |
| | | | | | N123R; | | | (SEQ | |
| | | | | | K124Y | | | ID | |
| | | | | | | | | NO: | |
| | | | | | | | | 33) | |
| 98 | | | | | hCPN1 | | TEV | GSSG | HSA |
| | | | | | (1-398); | | | GSSG | |
| | | | | | K124Q | | | GSSG | |
| | | | | | | | | (SEQ | |
| | | | | | | | | ID | |
| | | | | | | | | NO: | |
| | | | | | | | | 33) | |
| 99 | | | | | hCPN1 | | TEV | GSSG | HSA |
| | | | | | (1-398); | | | GSSG | |
| | | | | | G121T; | | | GSSG | |
| | | | | | P122Y; | | | (SEQ | |
| | | | | | K124Q; | | | ID | |
| | | | | | P125D; | | | NO: | |
| | | | | | E297P; | | | 33) | |
| | | | | | E298Q | | | | |
| 100 | | | | | hCPN1 | | TEV | GSSG | HSA |
| | | | | | (1-398); | | | GSSG | |
| | | | | | G121T; | | | GSSG | |
| | | | | | P122Y; | | | (SEQ | |
| | | | | | K124Q; | | | ID | |
| | | | | | P125D; | | | NO: | |
| | | | | | E297P | | | 33) | |
| 101 | | | | | hCPN1 | | TEV | GSSG | HSA |
| | | | | | (1-398); | | | GSSG | |
| | | | | | D292I; | | | GSSG | |
| | | | | | K293I; | | | (SEQ | |
| | | | | | F294W | | | ID | |
| | | | | | | | | NO: | |
| | | | | | | | | 33) | |
| 102 | | | | | hCPN1 | | TEV | GSSG | HSA |
| | | | | | (1-398); | | | GSSG | |
| | | | | | K293L; | | | GSSG | |
| | | | | | E297P; | | | (SEQ | |
| | | | | | E298Q | | | ID | |
| | | | | | | | | NO: | |
| | | | | | | | | 33) | |
| 103 | | | | | hCPN1 | | TEV | GSSG | HSA |
| | | | | | (1-398); | | | GSSG | |
| | | | | | D292L; | | | GSSG | |
| | | | | | K293I; | | | (SEQ | |
| | | | | | P295L | | | ID | |
| | | | | | | | | NO: | |
| | | | | | | | | 33) | |
| 104 | | | | | hCPN1 | | TEV | GSSG | HSA |
| | | | | | (1-398); | | | GSSG | |
| | | | | | D292L; | | | GSSG | |
| | | | | | K293I | | | (SEQ | |
| | | | | | | | | ID | |
| | | | | | | | | NO: | |
| | | | | | | | | 33) | |
| 105 | | | | | hCPN1 | | TEV | GSSG | HSA |
| | | | | | (1-398); | | | GSSG | |
| | | | | | D292N; | | | GSSG | |
| | | | | | K293A; | | | (SEQ | |
| | | | | | P295T; | | | ID | |
| | | | | | E298S | | | NO: | |
| | | | | | | | | 33) | |
| 106 | | | | | hCPN1 | | TEV | GSSG | HSA |
| | | | | | (1-398); | | | GSSG | |
| | | | | | D292N; | | | GSSG | |
| | | | | | K293A; | | | (SEQ | |
| | | | | | E298S | | | ID | |
| | | | | | | | | NO: | |
| | | | | | | | | 33) | |
| 107 | | | | | hCPN1 | | TEV | GSSG | HSA |
| | | | | | (1-398); | | | GSSG | |
| | | | | | D292I; | | | GSSG | |
| | | | | | K293A; | | | (SEQ | |
| | | | | | E294W | | | ID | |
| | | | | | | | | NO: | |
| | | | | | | | | 33) | |
| 108 | | | | | hCPN1 | | TEV | GSSG | HSA |
| | | | | | (1-398); | | | GSSG | |
| | | | | | D292L; | | | GSSG | |
| | | | | | K293A; | | | (SEQ | |
| | | | | | E294W | | | ID | |
| | | | | | | | | NO: | |
| | | | | | | | | 33) | |
| 109 | | | | | hCPN1 | | TEV | GSSG | HSA |
| | | | | | (1-398); | | | GSSG | |
| | | | | | D292L; | | | GSSG | |
| | | | | | K293V; | | | (SEQ | |
| | | | | | E295L | | | ID | |
| | | | | | | | | NO: | |
| | | | | | | | | 33) | |
| 110 | | | | | hCPN1 | | TEV | GSSG | HSA |
| | | | | | (1-398); | | | GSSG | |
| | | | | | D292L; | | | GSSG | |
| | | | | | K293V; | | | (SEQ | |
| | | | | | E298D | | | ID | |
| | | | | | | | | NO: | |
| | | | | | | | | 33) | |
| 111 | | | | | hCPN1 | | TEV | GSSG | HSA |
| | | | | | (1-398); | | | GSSG | |
| | | | | | K293N | | | GSSG | |
| | | | | | | | | (SEQ | |
| | | | | | | | | ID | |
| | | | | | | | | NO: | |
| | | | | | | | | 33) | |
| 112 | | | | | hCPN1 | | TEV | GSSG | HSA |
| | | | | | (1-398); | | | GSSG | |
| | | | | | K293L; | | | GSSG | |
| | | | | | F294W; | | | (SEQ | |
| | | | | | E298L | | | ID | |
| | | | | | | | | NO: | |
| | | | | | | | | 33) | |
| 113 | | | | | hCPN1 | | TEV | GSSG | HSA |
| | | | | | (1-398); | | | GSSG | |
| | | | | | D292N; | | | GSSG | |
| | | | | | K293L; | | | (SEQ | |
| | | | | | F294W | | | ID | |
| | | | | | | | | NO: | |
| | | | | | | | | 33) | |
| 114 | | | | | hCPN1 | | TEV | GSSG | HSA |
| | | | | | (1-398); | | | GSSG | |
| | | | | | N123W; | | | GSSG | |
| | | | | | K124R; | | | (SEQ | |
| | | | | | P125A; | | | ID | |
| | | | | | Y127D; | | | NO: | |
| | | | | | D292I; | | | 33) | |
| | | | | | K293I; | | | | |
| | | | | | F294Q | | | | |
| 115 | | | | | hCPN1 | | TEV | GSSG | HSA |
| | | | | | (1-398); | | | GSSG | |
| | | | | | N123Y; | | | GSSG | |
| | | | | | K124R; | | | (SEQ | |
| | | | | | P127E; | | | ID | |
| | | | | | D292I; | | | NO: | |
| | | | | | K293V; | | | 33) | |
| | | | | | F294M | | | | |
| 116 | | | | | hCPN1 | | TEV | GSSG | HSA |
| | | | | | (1-398); | | | GSSG | |
| | | | | | G121T; | | | GSSG | |
| | | | | | P122Y; | | | (SEQ | |
| | | | | | K124Q; | | | ID | |
| | | | | | P125D; | | | NO: | |
| | | | | | Y127W; | | | 33) | |
| | | | | | D292I; | | | | |
| | | | | | K293L; | | | | |
| | | | | | E297P | | | | |
| 117 | | | | | hCPN1 | | TEV | GSSG | HSA |
| | | | | | (1-398); | | | GSSG | |
| | | | | | N123Y; | | | GSSG | |
| | | | | | K124R; | | | (SEQ | |
| | | | | | Y127D; | | | ID | |
| | | | | | K293I | | | NO: | |
| | | | | | | | | 33) | |
| 118 | | | | | hCPN1 | | TEV | GSSG | HSA |
| | | | | | (1-398); | | | GSSG | |
| | | | | | G121D; | | | GSSG | |
| | | | | | P122S; | | | (SEQ | |
| | | | | | N123T; | | | ID | |
| | | | | | K124I; | | | NO: | |
| | | | | | D292N; | | | 33) | |
| | | | | | K293A; | | | | |
| | | | | | E298S | | | | |
| 119 | | | | | hCPN1 | | TEV | GSSG | HSA |
| | | | | | (1-398); | | | GSSG | |
| | | | | | G121Q; | | | GSSG | |
| | | | | | P122W; | | | (SEQ | |
| | | | | | N123D; | | | ID | |
| | | | | | Y127I; | | | NO: | |
| | | | | | D292L; | | | 33) | |
| | | | | | K293A; | | | | |
| | | | | | E294I | | | | |
| 120 | | | | | hCPN1 | | TEV | GSSG | HSA |
| | | | | | (1-398); | | | GSSG | |
| | | | | | G121D; | | | GSSG | |
| | | | | | P122W; | | | (SEQ | |
| | | | | | N123D; | | | ID | |
| | | | | | Y127I; | | | NO: | |
| | | | | | K293A; | | | 33) | |
| | | | | | E294W | | | | |
| 121 | | | | | hCPN1 | | TEV | GSSG | HSA |
| | | | | | (1-398); | | | GSSG | |
| | | | | | P122W; | | | GSSG | |
| | | | | | N123R; | | | (SEQ | |
| | | | | | K124H; | | | ID | |
| | | | | | Y127S; | | | NO: | |
| | | | | | D292L; | | | 33) | |
| | | | | | K293V; | | | | |
| | | | | | E298W | | | | |
| 122 | | | | | hCPN1 | | TEV | GSSG | HSA |
| | | | | | (1-398); | | | GSSG | |
| | | | | | P122W; | | | GSSG | |
| | | | | | N123R; | | | (SEQ | |
| | | | | | K124Y; | | | ID | |
| | | | | | D292L; | | | NO: | |
| | | | | | K293V; | | | 33) | |
| | | | | | E298D | | | | |
| 123 | | | | | hCPN1 | | TEV | GSSG | HSA |
| | | | | | (1-398); | | | GSSG | |
| | | | | | K124Q; | | | GSSG | |
| | | | | | K293L; | | | (SEQ | |
| | | | | | F294W; | | | ID | |
| | | | | | E298L | | | NO: | |
| | | | | | | | | 33) | |
| 124 | | | | | hCPN1 | | TEV | GSSG | HSA |
| | | | | | (1-398); | | | GSSG | |
| | | | | | K124D; | | | GSSG | |
| | | | | | K293L; | | | (SEQ | |
| | | | | | F294W; | | | ID | |
| | | | | | E297D | | | NO: | |
| | | | | | | | | 33) | |
| 125 | | | | | hCPN1 | | TEV | GSSG | HSA |
| | | | | | (1-398); | | | GSSG | |
| | | | | | P125H | | | GSSG | |
| | | | | | | | | (SEQ | |
| | | | | | | | | ID | |
| | | | | | | | | NO: | |
| | | | | | | | | 33) | |
| 126 | | | | | hCPN1 | | TEV | GSSG | HSA |
| | | | | | (1-398); | | | GSSG | |
| | | | | | P125L | | | GSSG | |
| | | | | | | | | (SEQ | |
| | | | | | | | | ID | |
| | | | | | | | | NO: | |
| | | | | | | | | 33) | |
| 127 | | | | | hCPN1 | | TEV | GSSG | HSA |
| | | | | | (1-398); | | | GSSG | |
| | | | | | P122A; | | | GSSG | |
| | | | | | N123D; | | | (SEQ | |
| | | | | | K124I; | | | ID | |
| | | | | | P125S | | | NO: | |
| | | | | | | | | 33) | |
| 128 | | | | | hCPN1 | | TEV | GSSG | HSA |
| | | | | | (1-398); | | | GSSG | |
| | | | | | P122A; | | | GSSG | |
| | | | | | N123D; | | | (SEQ | |
| | | | | | K124S; | | | ID | |
| | | | | | P125S | | | NO: | |
| | | | | | | | | 33) | |
| 129 | | | | | hCPN1 | | TEV | GSSG | HSA |
| | | | | | (1-398); | | | GSSG | |
| | | | | | G121E; | | | GSSG | |
| | | | | | P122R; | | | (SEQ | |
| | | | | | N123D; | | | ID | |
| | | | | | K124I; | | | NO: | |
| | | | | | P125S | | | 33) | |
| 130 | | | | | hCPN1 | | TEV | GSSG | HSA |
| | | | | | (1-398); | | | GSSG | |
| | | | | | G121D; | | | GSSG | |
| | | | | | P122R; | | | (SEQ | |
| | | | | | N123D; | | | ID | |
| | | | | | K124T; | | | NO: | |
| | | | | | P125S; | | | 33) | |
| | | | | | G126Q | | | | |
| 131 | | | | | hCPN1 | | TEV | GSSG | HSA |
| | | | | | (1-398); | | | GSSG | |
| | | | | | N123D; | | | GSSG | |
| | | | | | K124D; | | | (SEQ | |
| | | | | | P125S; | | | ID | |
| | | | | | G126Q | | | NO: | |
| | | | | | | | | 33) | |
| 132 | | | | | hCPN1 | | TEV | GSSG | HSA |
| | | | | | (1-398); | | | GSSG | |
| | | | | | N68D | | | GSSG | |
| | | | | | | | | (SEQ | |
| | | | | | | | | ID | |
| | | | | | | | | NO: | |
| | | | | | | | | 33) | |
| 133 | | | | | hCPN1 | | TEV | GSSG | HSA |
| | | | | | (1-398); | | | GSSG | |
| | | | | | N68K; | | | GSSG | |
| | | | | | K293N | | | (SEQ | |
| | | | | | | | | ID | |
| | | | | | | | | NO: | |
| | | | | | | | | 33) | |
| 134 | | | | | hCPN1 | | TEV | GSSG | HSA |
| | | | | | (1-398); | | | GSSG | |
| | | | | | V201H | | | GSSG | |
| | | | | | | | | (SEQ | |
| | | | | | | | | ID | |
| | | | | | | | | NO: | |
| | | | | | | | | 33) | |
| 135 | | | | | hCPN1 | | TEV | GSSG | HSA |
| | | | | | (1-398); | | | GSSG | |
| | | | | | N203D | | | GSSG | |
| | | | | | | | | (SEQ | |
| | | | | | | | | ID | |
| | | | | | | | | NO: | |
| | | | | | | | | 33) | |
| 136 | | | | | hCPN1 | | TEV | GSSG | HSA |
| | | | | | (1-398); | | | GSSG | |
| | | | | | G198D | | | GSSG | |
| | | | | | | | | (SEQ | |
| | | | | | | | | ID | |
| | | | | | | | | NO: | |
| | | | | | | | | 33) | |
| 137 | | | | | hCPN1 | | TEV | GSSG | HSA |
| | | | | | (1-398); | | | GSSG | |
| | | | | | G198D; | | | GSSG | |
| | | | | | V201Y | | | (SEQ | |
| | | | | | | | | ID | |
| | | | | | | | | NO: | |
| | | | | | | | | 33) | |
| 138 | | | | | hCPN1 | | TEV | GSSG | HSA |
| | | | | | (1-398); | | | GSSG | |
| | | | | | A70V | | | GSSG | |
| | | | | | | | | (SEQ | |
| | | | | | | | | ID | |
| | | | | | | | | NO: | |
| | | | | | | | | 33) | |
| 139 | | | | | hCPN1 | | TEV | GSSG | HSA |
| | | | | | (1-398); | | | GSSG | |
| | | | | | N68D; | | | GSSG | |
| | | | | | P125S | | | (SEQ | |
| | | | | | | | | ID | |
| | | | | | | | | NO: | |
| | | | | | | | | 33) | |
| 140 | | | | | hCPN1 | | TEV | GSSG | HSA |
| | | | | | (1-398); | | | GSSG | |
| | | | | | N68D; | | | GSSG | |
| | | | | | A70V; | | | (SEQ | |
| | | | | | P125S | | | ID | |
| | | | | | | | | NO: | |
| | | | | | | | | 33) | |
| 141 | | | | | hCPN1 | | | GSSG | HSA |
| | | | | | (1-398); | | | GSSG | |
| | | | | | E288Q | | | GSSG | |
| | | | | | | | | (SEQ | |
| | | | | | | | | ID | |
| | | | | | | | | NO: | |
| | | | | | | | | 33) | |
| 142 | | | | | hCPN1 | | | GSSG | HSA |
| | | | | | (1-398); | | | GSSG | |
| | | | | | Y266F | | | GSSG | |
| | | | | | | | | (SEQ | |
| | | | | | | | | ID | |
| | | | | | | | | NO: | |
| | | | | | | | | 33) | |
| 143 | | | | | hCPN1 | | | GSSG | HSA |
| | | | | | (1-398); | | | GSSG | |
| | | | | | Y266F; | | | GSSG | |
| | | | | | E288Q | | | (SEQ | |
| | | | | | | | | ID | |
| | | | | | | | | NO: | |
| | | | | | | | | 33) | |
| 144 | | | | | hCPN1 | | | GSSG | HSA |
| | | | | | (1-398); | | | GSSG | |
| | | | | | A199S | | | GSSG | |
| | | | | | | | | (SEQ | |
| | | | | | | | | ID | |
| | | | | | | | | NO: | |
| | | | | | | | | 33) | |
| 145 | | | | | hCPN1 | | | GSSG | HSA |
| | | | | | (1-398); | | | GSSG | |
| | | | | | K124R; | | | GSSG | |
| | | | | | P125S | | | (SEQ | |
| | | | | | | | | ID | |
| | | | | | | | | NO: | |
| | | | | | | | | 33) | |
| 146 | | | | | hCPN1 | | | GSSG | HSA |
| | | | | | (1-398); | | | GSSG | |
| | | | | | G121T; | | | GSSG | |
| | | | | | P122Y; | | | (SEQ | |
| | | | | | K124Q; | | | ID | |
| | | | | | P125D | | | NO: | |
| | | | | | | | | 33) | |
| 147 | | | | | hCPN1 | | | GSSG | HSA |
| | | | | | (1-398); | | | GSSG | |
| | | | | | N123Y; | | | GSSG | |
| | | | | | K124R | | | (SEQ | |
| | | | | | | | | ID | |
| | | | | | | | | NO: | |
| | | | | | | | | 33) | |
| 148 | | | | | hCPN1 | | | GSSG | HSA |
| | | | | | (1-398); | | | GSSG | |
| | | | | | G121D; | | | GSSG | |
| | | | | | P122D; | | | (SEQ | |
| | | | | | N123T; | | | ID | |
| | | | | | K124I | | | NO: | |
| | | | | | | | | 33) | |
| 149 | | | | | hCPN1 | | | GSSG | HSA |
| | | | | | (1-398); | | | GSSG | |
| | | | | | G121D; | | | GSSG | |
| | | | | | P122H; | | | (SEQ | |
| | | | | | K124I | | | ID | |
| | | | | | | | | NO: | |
| | | | | | | | | 33) | |
| 150 | | | | | hCPN1 | | | GSSG | HSA |
| | | | | | (1-398); | | | GSSG | |
| | | | | | G121R; | | | GSSG | |
| | | | | | P122E; | | | (SEQ | |
| | | | | | N123Y; | | | ID | |
| | | | | | K124I | | | NO: | |
| | | | | | | | | 33) | |
| 151 | | | | | hCPN1 | | | GSSG | HSA |
| | | | | | (1-398); | | | GSSG | |
| | | | | | G121D; | | | GSSG | |
| | | | | | P122H; | | | (SEQ | |
| | | | | | K124Q | | | ID | |
| | | | | | | | | NO: | |
| | | | | | | | | 33) | |
| 152 | | | | | hCPN1 | | | GSSG | HSA |
| | | | | | (1-398); | | | GSSG | |
| | | | | | G121I; | | | GSSG | |
| | | | | | N123D | | | (SEQ | |
| | | | | | | | | ID | |
| | | | | | | | | NO: | |
| | | | | | | | | 33) | |
| 153 | | | | | hCPN1 | | TEV | GSSG | HSA |
| | | | | | (1-320); | | | GSSG | |
| | | | | | L55N; | | | GSSG | |
| | | | | | H161R; | | | (SEQ | |
| | | | | | N188D; | | | ID | |
| | | | | | V190K; | | | NO: | |
| | | | | | K229E; | | | 33) | |
| | | | | | L230W; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 154 | | | | | hCPN1 | | TEV | GSSG | HSA |
| | | | | | (1-320); | | | GSSG | |
| | | | | | L55N; | | | GSSG | |
| | | | | | H161S; | | | (SEQ | |
| | | | | | N188D; | | | ID | |
| | | | | | V190R; | | | NO: | |
| | | | | | K229E; | | | 33) | |
| | | | | | L230W; | | | | |
| | | | | | C282S; | | | | |
| | | | | | F283Y; | | | | |
| | | | | | R308K; | | | | |
| | | | | | Q320N | | | | |
| 155 | | | | | hCPN1 | | TEV | GSSG | HSA |
| | | | | | (1-320); | | | GSSG | |
| | | | | | L55N; | | | GSSG | |
| | | | | | H161K; | | | (SEQ | |
| | | | | | N188D; | | | ID | |
| | | | | | V190K; | | | NO: | |
| | | | | | K229E; | | | 33) | |
| | | | | | L230W; | | | | |
| | | | | | C282S; | | | | |
| | | | | | F283Y; | | | | |
| | | | | | R308K; | | | | |
| | | | | | Q320N | | | | |
| 156 | | | | | hCPN1 | | | GSSG | HSA |
| | | | | | (1-398); | | | GSSG | |
| | | | | | P122W; | | | GSSG | |
| | | | | | N123R; | | | (SEQ | |
| | | | | | K124Y | | | ID | |
| | | | | | | | | NO: | |
| | | | | | | | | 33) | |
| 157 | | | | | hCPN1 | | | GSSG | HSA |
| | | | | | (1-398); | | | GSSG | |
| | | | | | P122Y; | | | GSSG | |
| | | | | | N123R; | | | (SEQ | |
| | | | | | K124Y | | | ID | |
| | | | | | | | | NO: | |
| | | | | | | | | 33) | |
| 158 | | | | | hCPN1 | | | GSSG | HSA |
| | | | | | (1-398); | | | GSSG | |
| | | | | | K124Q | | | GSSG | |
| | | | | | | | | (SEQ | |
| | | | | | | | | ID | |
| | | | | | | | | NO: | |
| | | | | | | | | 33) | |
| 159 | | | | | hCPN1 | | | GSSG | HSA |
| | | | | | (1-398); | | | GSSG | |
| | | | | | G121D; | | | GSSG | |
| | | | | | | | | (SEQ | |
| | | | | | | | | ID | |
| | | | | | | | | NO: | |
| | | | | | | | | 33) | |
| 160 | | | | | hCPN1 | | | GSSG | HSA |
| | | | | | (1-398); | | | GSSG | |
| | | | | | N123D; | | | GSSG | |
| | | | | | | | | (SEQ | |
| | | | | | | | | ID | |
| | | | | | | | | NO: | |
| | | | | | | | | 33) | |
| 161 | | | | | hCPN1 | | | GSSG | HSA |
| | | | | | (1-398); | | | GSSG | |
| | | | | | G121D; | | | GSSG | |
| | | | | | N123D; | | | (SEQ | |
| | | | | | | | | ID | |
| | | | | | | | | NO: | |
| | | | | | | | | 33) | |
| 162 | | | | | hCPN1 | | | GSSG | HSA |
| | | | | | (1-398); | | | GSSG | |
| | | | | | P122A; | | | GSSG | |
| | | | | | | | | (SEQ | |
| | | | | | | | | ID | |
| | | | | | | | | NO: | |
| | | | | | | | | 33) | |
| 163 | | | | | hCPN1 | | | GSSG | HSA |
| | | | | | (1-398); | | | GSSG | |
| | | | | | P122R; | | | GSSG | |
| | | | | | | | | (SEQ | |
| | | | | | | | | ID | |
| | | | | | | | | NO: | |
| | | | | | | | | 33) | |
| 164 | | | | | hCPN1 | | | GSSG | HSA |
| | | | | | (1-398); | | | GSSG | |
| | | | | | K124I; | | | GSSG | |
| | | | | | | | | (SEQ | |
| | | | | | | | | ID | |
| | | | | | | | | NO: | |
| | | | | | | | | 33) | |
| 165 | | | | | hCPN1 | | | GSSG | HSA |
| | | | | | (1-398); | | | GSSG | |
| | | | | | G121D; | | | GSSG | |
| | | | | | N123D; | | | (SEQ | |
| | | | | | K124I; | | | ID | |
| | | | | | | | | NO: | |
| | | | | | | | | 33) | |
| 166 | | | | | hCPN1 | | | GSSG | HSA |
| | | | | | (1-398); | | | GSSG | |
| | | | | | P125S; | | | GSSG | |
| | | | | | | | | (SEQ | |
| | | | | | | | | ID | |
| | | | | | | | | NO: | |
| | | | | | | | | 33) | |
| 167 | | | | | hCPN1 | | | GSSG | HSA |
| | | | | | (1-398); | | | GSSG | |
| | | | | | P122G; | | | GSSG | |
| | | | | | | | | (SEQ | |
| | | | | | | | | ID | |
| | | | | | | | | NO: | |
| | | | | | | | | 33) | |
| 168 | | | | | hCPN1 | | | GSSG | HSA |
| | | | | | (1-398); | | | GSSG | |
| | | | | | K124S; | | | GSSG | |
| | | | | | | | | (SEQ | |
| | | | | | | | | ID | |
| | | | | | | | | NO: | |
| | | | | | | | | 33) | |
| 169 | | | | | hCPN1 | | | GSSG | HSA |
| | | | | | (1-398); | | | GSSG | |
| | | | | | K124G; | | | GSSG | |
| | | | | | | | | (SEQ | |
| | | | | | | | | ID | |
| | | | | | | | | NO: | |
| | | | | | | | | 33) | |
| 170 | | | | | hCPN1 | | | GSSG | HSA |
| | | | | | (1-398); | | | GSSG | |
| | | | | | P125N; | | | GSSG | |
| | | | | | | | | (SEQ | |
| | | | | | | | | ID | |
| | | | | | | | | NO: | |
| | | | | | | | | 33) | |
| 171 | | | | | hCPN1 | | | GSSG | HSA |
| | | | | | (1-398); | | | GSSG | |
| | | | | | P125G; | | | GSSG | |
| | | | | | | | | (SEQ | |
| | | | | | | | | ID | |
| | | | | | | | | NO: | |
| | | | | | | | | 33) | |
| 172 | | | | | hCPN1 | | | GSSG | HSA |
| | | | | | (1-398); | | | GSSG | |
| | | | | | E297Q; | | | GSSG | |
| | | | | | | | | (SEQ | |
| | | | | | | | | ID | |
| | | | | | | | | NO: | |
| | | | | | | | | 33) | |
| 173 | | | | | hCPN1 | | | GSSG | HSA |
| | | | | | (1-398); | | | GSSG | |
| | | | | | V180F; | | | GSSG | |
| | | | | | | | | (SEQ | |
| | | | | | | | | ID | |
| | | | | | | | | NO: | |
| | | | | | | | | 33) | |
| 174 | | | | | hCPN1 | | | GSSG | HSA |
| | | | | | (1-398); | | | GSSG | |
| | | | | | S192A; | | | GSSG | |
| | | | | | | | | (SEQ | |
| | | | | | | | | ID | |
| | | | | | | | | NO: | |
| | | | | | | | | 33) | |
| 175 | | | | | hCPN1 | | | GSSG | HSA |
| | | | | | (1-398); | | | GSSG | |
| | | | | | N276V; | | | GSSG | |
| | | | | | | | | (SEQ | |
| | | | | | | | | ID | |
| | | | | | | | | NO: | |
| | | | | | | | | 33) | |
| 176 | | | | | hCPN1 | | | GSSG | HSA |
| | | | | | (1-398); | | | GSSG | |
| | | | | | E284I; | | | GSSG | |
| | | | | | | | | (SEQ | |
| | | | | | | | | ID | |
| | | | | | | | | NO: | |
| | | | | | | | | 33) | |
| 177 | | | | | hCPN1 | | | GSSG | HSA |
| | | | | | (1-398); | | | GSSG | |
| | | | | | K60F; | | | GSSG | |
| | | | | | | | | (SEQ | |
| | | | | | | | | ID | |
| | | | | | | | | NO: | |
| | | | | | | | | 33) | |
| 178 | mMBP | AAAQ | TEV | | hCPN1 | | | | |
| | | TNAA | | | (1-398); | | | | |
| | | AASS | | | Y266F; | | | | |
| | | ASLE | | | E288Q | | | | |
| | | (SEQ | | | | | | | |
| | | ID | | | | | | | |
| | | NO: | | | | | | | |
| | | 35) | | | | | | | |
| 179 | mMBP | AAAQ | TEV | | hCPN1 | | TEV | GSSG | HSA |
| | | TNAA | | | (1-398); | | | GSSG | |
| | | AASS | | | Y266F; | | | GSSG | |
| | | ASLE | | | E288Q | | | (SEQ | |
| | | (SEQ | | | | | | ID | |
| | | ID | | | | | | NO: | |
| | | NO: | | | | | | 33) | |
| | | 35) | | | | | | | |
| 180 | mMBP | AAAQ | TEV | | hCPN1 | | | | |
| | | TNAA | | | (1-438) | | | | |
| | | AASS | | | | | | | |
| | | ASLE | | | | | | | |
| | | (SEQ | | | | | | | |
| | | ID | | | | | | | |
| | | NO: | | | | | | | |
| | | 35) | | | | | | | |
| 181 | mMBP | AAAQ | TEV | | hCPN1 | | TEV | GSSG | HSA |
| | | TNAA | | | (1-438) | | | GSSG | |
| | | AASS | | | | | | GSSG | |
| | | ASLE | | | | | | (SEQ | |
| | | (SEQ | | | | | | ID | |
| | | ID | | | | | | NO: | |
| | | NO: | | | | | | 33) | |
| | | 35) | | | | | | | |
| 182 | mMBP | AAAQ | TEV | | hCPN1 | | | | |
| | | TNAA | | | (1-438); | | | | |
| | | AASS | | | Y266F; | | | | |
| | | ASLE | | | E288Q | | | | |
| | | (SEQ | | | | | | | |
| | | ID | | | | | | | |
| | | NO: | | | | | | | |
| | | 35) | | | | | | | |
| 183 | mMBP | AAAQ | TEV | | hCPN1 | | TEV | GSSG | HSA |
| | | TNAA | | | (1-438); | | | GSSG | |
| | | AASS | | | Y266F; | | | GSSG | |
| | | ASLE | | | E288Q | | | (SEQ | |
| | | (SEQ | | | | | | ID | |
| | | ID | | | | | | NO: | |
| | | NO: | | | | | | 33) | |
| | | 35) | | | | | | | |
| 184 | GST | AAAQ | TEV | | hCPN1 | | TEV | GSSG | HSA |
| | | TNAA | | | (1-398); | | | GSSG | |
| | | AASS | | | Y266F; | | | GSSG | |
| | | ASLE | | | E288Q | | | (SEQ | |
| | | (SEQ | | | | | | ID | |
| | | ID | | | | | | NO: | |
| | | NO: | | | | | | 33) | |
| | | 35) | | | | | | | |
| 185 | GST | AAAQ | TEV | | hCPN1 | | | | |
| | | TNAA | | | (1-398) | | | | |
| | | AASS | | | | | | | |
| | | ASLE | | | | | | | |
| | | (SEQ | | | | | | | |
| | | ID | | | | | | | |
| | | NO: | | | | | | | |
| | | 35) | | | | | | | |
| 186 | GST | AAAQ | TEV | | hCPN1 | | | | |
| | | TNAA | | | (1-398); | | | | |
| | | AASS | | | Y266F; | | | | |
| | | ASLE | | | E288Q | | | | |
| | | (SEQ | | | | | | | |
| | | ID | | | | | | | |
| | | NO: | | | | | | | |
| | | 35) | | | | | | | |
| 187 | | | | | hCPN1 | | TEV | GSSG | HSA |
| | | | | | (1-438); | | | GSSG | |
| | | | | | Y266F; | | | GSSG | |
| | | | | | E288Q; | | | (SEQ | |
| | | | | | 398DDDD | | | ID | |
| | | | | | K399 | | | NO: | |
| | | | | | | | | 33) | |
| 188 | | | | | hCPN1 | | TEV | GSSG | HSA |
| | | | | | (1-438); | | | GSSG | |
| | | | | | Y266F; | | | GSSG | |
| | | | | | E288Q | | | (SEQ | |
| | | | | | | | | ID | |
| | | | | | | | | NO: | |
| | | | | | | | | 33) | |
| 189 | | | | | hCPN1 | | | GSSG | HSA |
| | | | | | (1-438); | | | GSSG | |
| | | | | | Y266F; | | | GSSG | |
| | | | | | E288Q | | | (SEQ | |
| | | | | | | | | ID | |
| | | | | | | | | NO: | |
| | | | | | | | | 33) | |
| 190 | | | | | hCPN1 | | | GSSG | HSA |
| | | | | | (1-320); | | | GSSG | |
| | | | | | L55N; | | | GSSG | |
| | | | | | H161R; | | | (SEQ | |
| | | | | | N188D; | | | ID | |
| | | | | | V190K; | | | NO: | |
| | | | | | K229E; | | | 33) | |
| | | | | | L230W; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 191 | | | | | hCPN1 | | | GSSG | HSA |
| | | | | | (1-320); | | | GSSG | |
| | | | | | L55N; | | | GSSG | |
| | | | | | H161S; | | | (SEQ | |
| | | | | | N188D; | | | ID | |
| | | | | | V190R; | | | NO: | |
| | | | | | K229E; | | | 33) | |
| | | | | | L230W; | | | | |
| | | | | | C282S; | | | | |
| | | | | | F283Y; | | | | |
| | | | | | R308K; | | | | |
| | | | | | Q320N | | | | |
| 192 | | | | | hCPN1 | | | GSSG | HSA |
| | | | | | (1-320); | | | GSSG | |
| | | | | | L55N; | | | GSSG | |
| | | | | | H161K; | | | (SEQ | |
| | | | | | N188D; | | | ID | |
| | | | | | V190K; | | | NO: | |
| | | | | | K229E; | | | 33) | |
| | | | | | L230W; | | | | |
| | | | | | C282S; | | | | |
| | | | | | F283Y; | | | | |
| | | | | | R308K; | | | | |
| | | | | | Q320N | | | | |
| 193 | | | | | hCPN1 | | | GSSG | HSA |
| | | | | | (1-320); | | | GSSG | |
| | | | | | L55N; | | | GSSG | |
| | | | | | H161S; | | | (SEQ | |
| | | | | | N188D; | | | ID | |
| | | | | | V190R; | | | NO: | |
| | | | | | K229E; | | | 33) | |
| | | | | | L230K; | | | | |
| | | | | | K233D; | | | | |
| | | | | | C282S; | | | | |
| | | | | | F283Y; | | | | |
| | | | | | R308K; | | | | |
| | | | | | Q320K | | | | |
| 194 | | | | | hCPN1 | | | GSSG | HSA |
| | | | | | (1-320); | | | GSSG | |
| | | | | | L55N; | | | GSSG | |
| | | | | | H161R; | | | (SEQ | |
| | | | | | N188D; | | | ID | |
| | | | | | V190K; | | | NO: | |
| | | | | | K229E; | | | 33) | |
| | | | | | L230W; | | | | |
| | | | | | K233H; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 195 | | | | | hCPN1 | | | GSSG | HSA |
| | | | | | (1-320); | | | GSSG | |
| | | | | | L55N; | | | GSSG | |
| | | | | | H161R; | | | (SEQ | |
| | | | | | N188D; | | | ID | |
| | | | | | V190K; | | | NO: | |
| | | | | | K229E; | | | 33) | |
| | | | | | L230K; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 196 | | | | | hCPN1 | | | GSSG | HSA |
| | | | | | (1-398); | | | GSSG | |
| | | | | | A199D; | | | GSSG | |
| | | | | | | | | (SEQ | |
| | | | | | | | | ID | |
| | | | | | | | | NO: | |
| | | | | | | | | 33) | |
| 197 | | | | | hCPN1 | | | GSSG | HSA |
| | | | | | (1-398); | | | GSSG | |
| | | | | | G121D; | | | GSSG | |
| | | | | | A199D; | | | (SEQ | |
| | | | | | | | | ID | |
| | | | | | | | | NO: | |
| | | | | | | | | 33) | |
| 198 | | | | | hCPN1 | | | GSSG | HSA |
| | | | | | (1-398); | | | GSSG | |
| | | | | | N123D; | | | GSSG | |
| | | | | | A199D; | | | (SEQ | |
| | | | | | | | | ID | |
| | | | | | | | | NO: | |
| | | | | | | | | 33) | |
| 199 | | | | | hCPN1 | | | GSSG | HSA |
| | | | | | (1-398); | | | GSSG | |
| | | | | | G121D; | | | GSSG | |
| | | | | | N123D; | | | (SEQ | |
| | | | | | A199D; | | | ID | |
| | | | | | | | | NO: | |
| | | | | | | | | 33) | |
| 200 | | | | | hCPN1 | | | GSSG | HSA |
| | | | | | (1-398); | | | GSSG | |
| | | | | | G121D; | | | GSSG | |
| | | | | | N123D; | | | (SEQ | |
| | | | | | K124I; | | | ID | |
| | | | | | A199D; | | | NO: | |
| | | | | | | | | 33) | |
| 201 | | | | | hCPN1 | | | GSSG | HSA |
| | | | | | (1-398); | | | GSSG | |
| | | | | | H66K; | | | GSSG | |
| | | | | | | | | (SEQ | |
| | | | | | | | | ID | |
| | | | | | | | | NO: | |
| | | | | | | | | 33) | |
| 202 | | | | | hCPN1 | | | GSSG | HSA |
| | | | | | (1-398); | | | GSSG | |
| | | | | | Q78E; | | | GSSG | |
| | | | | | | | | (SEQ | |
| | | | | | | | | ID | |
| | | | | | | | | NO: | |
| | | | | | | | | 33) | |
| 203 | | | | | hCPN1 | | | GSSG | HSA |
| | | | | | (1-398); | | | GSSG | |
| | | | | | P125E; | | | GSSG | |
| | | | | | | | | (SEQ | |
| | | | | | | | | ID | |
| | | | | | | | | NO: | |
| | | | | | | | | 33) | |
| 204 | | | | | hCPN1 | | | GSSG | HSA |
| | | | | | (1-398); | | | GSSG | |
| | | | | | P125D; | | | GSSG | |
| | | | | | | | | (SEQ | |
| | | | | | | | | ID | |
| | | | | | | | | NO: | |
| | | | | | | | | 33) | |
| 205 | | | | | hCPN1 | | | GSSG | HSA |
| | | | | | (1-398); | | | GSSG | |
| | | | | | D138K; | | | GSSG | |
| | | | | | | | | (SEQ | |
| | | | | | | | | ID | |
| | | | | | | | | NO: | |
| | | | | | | | | 33) | |
| 206 | | | | | hCPN1 | | | GSSG | HSA |
| | | | | | (1-398); | | | GSSG | |
| | | | | | P296E; | | | GSSG | |
| | | | | | | | | (SEQ | |
| | | | | | | | | ID | |
| | | | | | | | | NO: | |
| | | | | | | | | 33) | |
| 207 | | | | | hCPN1 | | | GSSG | HSA |
| | | | | | (1-398); | | | GSSG | |
| | | | | | P296G; | | | GSSG | |
| | | | | | | | | (SEQ | |
| | | | | | | | | ID | |
| | | | | | | | | NO: | |
| | | | | | | | | 33) | |
| 208 | | | | | hCPN1 | | | GSSG | HSA |
| | | | | | (1-398); | | | GSSG | |
| | | | | | E298G; | | | GSSG | |
| | | | | | | | | (SEQ | |
| | | | | | | | | ID | |
| | | | | | | | | NO: | |
| | | | | | | | | 33) | |
| 209 | | | | | hCPN1 | | | GSSG | HSA |
| | | | | | (1-398); | | | GSSG | |
| | | | | | L300E; | | | GSSG | |
| | | | | | | | | (SEQ | |
| | | | | | | | | ID | |
| | | | | | | | | NO: | |
| | | | | | | | | 33) | |
| 210 | | | | | hCPN1 | | | GSSG | HSA |
| | | | | | (1-398); | | | GSSG | |
| | | | | | Q301R; | | | GSSG | |
| | | | | | | | | (SEQ | |
| | | | | | | | | ID | |
| | | | | | | | | NO: | |
| | | | | | | | | 33) | |
| 211 | | | | | hCPN1 | | | GSSG | HSA |
| | | | | | (1-398); | | | GSSG | |
| | | | | | Q301E; | | | GSSG | |
| | | | | | | | | (SEQ | |
| | | | | | | | | ID | |
| | | | | | | | | NO: | |
| | | | | | | | | 33) | |
| 212 | | | | | hCPN1 | | | GSSG | HSA |
| | | | | | (1-398); | | | GSSG | |
| | | | | | R302E; | | | GSSG | |
| | | | | | | | | (SEQ | |
| | | | | | | | | ID | |
| | | | | | | | | NO: | |
| | | | | | | | | 33) | |
| 213 | | | | | hCPN1 | | | GSSG | HSA |
| | | | | | (1-398); | | | GSSG | |
| | | | | | D292N; | | | GSSG | |
| | | | | | | | | (SEQ | |
| | | | | | | | | ID | |
| | | | | | | | | NO: | |
| | | | | | | | | 33) | |
| 214 | | | | | hCPN1 | | | GSSG | HSA |
| | | | | | (1-398); | | | GSSG | |
| | | | | | F189C; | | | GSSG | |
| | | | | | | | | (SEQ | |
| | | | | | | | | ID | |
| | | | | | | | | NO: | |
| | | | | | | | | 33) | |
| 215 | | | | | hCPN1 | GSSG | HSA | GGGG | HHHH |
| | | | | | (1-398) | GSSG | | (SEQ | HHHH |
| | | | | | | GSSG | | ID | HH |
| | | | | | | (SEQ | | NO: | (SEQ |
| | | | | | | ID | | 39) | ID |
| | | | | | | NO: | | | NO: |
| | | | | | | 33) | | | 40) |
| 216 | | | | | aCPN1 | | | GSSG | HSA |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 33) | |
| 217 | | | | | bCPN1 | | | GSSG | HSA |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 33) | |
| 218 | | | | | mCPN1 | | | GSSG | HSA |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 33) | |
| 219 | | | | | vCPN1 | | | GSSG | HSA |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 33) | |
| 220 | | | | | uCPN1 | | | GSSG | HSA |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 33) | |
| 221 | | | | | hCPN1 | | | GSSG | HSA |
| | | | | | (1-398); | | | GSSG | |
| | | | | | R308K; | | | GSSG | |
| | | | | | E329Q; | | | (SEQ | |
| | | | | | | | | ID | |
| | | | | | | | | NO: | |
| | | | | | | | | 33) | |
| 222 | | | | | hCPN1 | | | | HSA |
| | | | | | (1-398); | | | | |
| 223 | | | | | hCPN1 | | | GSSG | HSA |
| | | | | | (1-398); | | | (SEQ | |
| | | | | | | | | ID | |
| | | | | | | | | NO: | |
| | | | | | | | | 41) | |
| 224 | | | | | hCPN1 | | | GSSG | HSA |
| | | | | | (1-398); | | | GSSG | |
| | | | | | | | | (SEQ | |
| | | | | | | | | ID | |
| | | | | | | | | NO: | |
| | | | | | | | | 42) | |
| 225 | | | | | hCPN1 | | | GSSG | HSA |
| | | | | | (1-398); | | | GSSG | |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 43) | |
| 226 | | | | | hCPN1 | | TEV | | HSA |
| | | | | | (1-398); | | | | |
| 227 | | | | | hCPN1 | | TEV | GSSG | HSA |
| | | | | | (1-398); | | | (SEQ | |
| | | | | | | | | ID | |
| | | | | | | | | NO: | |
| | | | | | | | | 41) | |
| 228 | | | | | hCPN1 | | TEV | GSSG | HSA |
| | | | | | (1-398); | | | GSSG | |
| | | | | | | | | (SEQ | |
| | | | | | | | | ID | |
| | | | | | | | | NO: | |
| | | | | | | | | 42) | |
| 229 | | | | | hCPN1 | | TEV | GSSG | HSA |
| | | | | | (1-398); | | | GSSG | |
| | | | | | | | | GSSG | |
| | | | | | | | | GSSG | |
| | | | | | | | | (SEQ | |
| | | | | | | | | ID | |
| | | | | | | | | NO: | |
| | | | | | | | | 43) | |
| 230 | | | | | hCPN1 | | | GSSG | HSA |
| | | | | | (1-398); | | | GSSG | |
| | | | | | A70V; | | | GSSG | |
| | | | | | P125S; | | | (SEQ | |
| | | | | | | | | ID | |
| | | | | | | | | NO: | |
| | | | | | | | | 33) | |
| 231 | | | | | hCPN1 | | | GSSG | HSA |
| | | | | | (1-398); | | | GSSG | |
| | | | | | P125S; | | | GSSG | |
| | | | | | | | | (SEQ | |
| | | | | | | | | ID | |
| | | | | | | | | NO: | |
| | | | | | | | | 33) | |
| 232 | | | | | hCPN1 | | | GSSG | HSA |
| | | | | | (1-398); | | | GSSG | |
| | | | | | A70V; | | | GSSG | |
| | | | | | P125N; | | | (SEQ | |
| | | | | | | | | ID | |
| | | | | | | | | NO: | |
| | | | | | | | | 33) | |
| 233 | | | | | hCPN1 | | | GSSG | HSA |
| | | | | | (1-398); | | | GSSG | |
| | | | | | A70V; | | | GSSG | |
| | | | | | P125I; | | | (SEQ | |
| | | | | | | | | ID | |
| | | | | | | | | NO: | |
| | | | | | | | | 33) | |
| 234 | | | | | hCPN1 | | | GSSG | HSA |
| | | | | | (1-398); | | | GSSG | |
| | | | | | A70V; | | | GSSG | |
| | | | | | P125F; | | | (SEQ | |
| | | | | | | | | ID | |
| | | | | | | | | NO: | |
| | | | | | | | | 33) | |
| 235 | | | | | hCPN1 | | | GSSG | HSA |
| | | | | | (1-398); | | | GSSG | |
| | | | | | N68D; | | | GSSG | |
| | | | | | A70V; | | | (SEQ | |
| | | | | | | | | ID | |
| | | | | | | | | NO: | |
| | | | | | | | | 33) | |
| 236 | | | | | hCPN1 | | | GSSG | HSA |
| | | | | | (1-398); | | | GSSG | |
| | | | | | N68D; | | | GSSG | |
| | | | | | A70V; | | | (SEQ | |
| | | | | | P125Y; | | | ID | |
| | | | | | | | | NO: | |
| | | | | | | | | 33) | |
| 237 | | | | | hCPN1 | | | GSSG | HSA |
| | | | | | (1-398); | | | GSSG | |
| | | | | | N68D; | | | GSSG | |
| | | | | | P125Y; | | | (SEQ | |
| | | | | | | | | ID | |
| | | | | | | | | NO: | |
| | | | | | | | | 33) | |
| 238 | | | | | hCPN1 | | | GSSG | HSA |
| | | | | | (1-398); | | | GSSG | |
| | | | | | N123D; | | | GSSG | |
| | | | | | K124S; | | | (SEQ | |
| | | | | | P125N; | | | ID | |
| | | | | | V129T; | | | NO: | |
| | | | | | | | | 33) | |
| 239 | | | | | hCPN1 | | | GSSG | HSA |
| | | | | | (1-398); | | | GSSG | |
| | | | | | K124T; | | | GSSG | |
| | | | | | P125S; | | | (SEQ | |
| | | | | | | | | ID | |
| | | | | | | | | NO: | |
| | | | | | | | | 33) | |
| 240 | | | | | hCPN1 | | | GSSG | HSA |
| | | | | | (1-398); | | | GSSG | |
| | | | | | G121D; | | | GSSG | |
| | | | | | P122R; | | | (SEQ | |
| | | | | | N123D; | | | ID | |
| | | | | | K124I; | | | NO: | |
| | | | | | P125S; | | | 33) | |
| 241 | | | | | hCPN1 | | | GSSG | HSA |
| | | | | | (1-398); | | | GSSG | |
| | | | | | N123D; | | | GSSG | |
| | | | | | K124I; | | | (SEQ | |
| | | | | | P125S; | | | ID | |
| | | | | | | | | NO: | |
| | | | | | | | | 33) | |
| 242 | | | | | hCPN1 | | | GSSG | HSA |
| | | | | | (1-398); | | | GSSG | |
| | | | | | N123D; | | | GSSG | |
| | | | | | K124S; | | | (SEQ | |
| | | | | | P125I; | | | ID | |
| | | | | | V129T; | | | NO: | |
| | | | | | | | | 33) | |
| 243 | | | | | hCPN1 | | | GSSG | HSA |
| | | | | | (1-398); | | | GSSG | |
| | | | | | N123D; | | | GSSG | |
| | | | | | K124A; | | | (SEQ | |
| | | | | | P125S; | | | ID | |
| | | | | | | | | NO: | |
| | | | | | | | | 33) | |
| 244 | | | | | hCPN1 | | | GSSG | HSA |
| | | | | | (1-398); | | | GSSG | |
| | | | | | K124A; | | | GSSG | |
| | | | | | P125S; | | | (SEQ | |
| | | | | | | | | ID | |
| | | | | | | | | NO: | |
| | | | | | | | | 33) | |
| 245 | | | | | hCPN1 | | | GSSG | HSA |
| | | | | | (1-398); | | | GSSG | |
| | | | | | N123D; | | | GSSG | |
| | | | | | K124A; | | | (SEQ | |
| | | | | | P125N; | | | ID | |
| | | | | | V129T; | | | NO: | |
| | | | | | | | | 33) | |
| 246 | | | | | hCPN1 | | | GSSG | HSA |
| | | | | | (1-398); | | | GSSG | |
| | | | | | G121E; | | | GSSG | |
| | | | | | P122R; | | | (SEQ | |
| | | | | | N123D; | | | ID | |
| | | | | | K124V; | | | NO: | |
| | | | | | P125S; | | | 33) | |
| 247 | | | | | hCPN1 | | | GSSG | HSA |
| | | | | | (1-398); | | | GSSG | |
| | | | | | N123S; | | | GSSG | |
| | | | | | K124T; | | | (SEQ | |
| | | | | | P125S; | | | ID | |
| | | | | | | | | NO: | |
| | | | | | | | | 33) | |
| 248 | | | | | hCPN1 | | | GSSG | HSA |
| | | | | | (1-398); | | | GSSG | |
| | | | | | K60F; | | | GSSG | |
| | | | | | V62I; | | | (SEQ | |
| | | | | | V180I; | | | ID | |
| | | | | | M184L; | | | NO: | |
| | | | | | | | | 33) | |
| | | | | | S192A; | | | | |
| | | | | | M272L; | | | | |
| | | | | | N276I; | | | | |
| | | | | | E284V; | | | | |
| 249 | | | | | hCPN1 | | | GSSG | HSA |
| | | | | | (1-398); | | | GSSG | |
| | | | | | K60F; | | | GSSG | |
| | | | | | V62A; | | | (SEQ | |
| | | | | | V180F; | | | ID | |
| | | | | | M184L; | | | NO: | |
| | | | | | S192A; | | | 33) | |
| | | | | | M272A; | | | | |
| | | | | | N276V; | | | | |
| | | | | | E284I; | | | | |
| 250 | | | | | hCPN1 | | | GSSG | HSA |
| | | | | | (1-398); | | | GSSG | |
| | | | | | K60F; | | | GSSG | |
| | | | | | V62T; | | | (SEQ | |
| | | | | | V180F; | | | ID | |
| | | | | | S192A; | | | NO: | |
| | | | | | N276V; | | | 33) | |
| | | | | | E284I; | | | | |
| 251 | | | | | hCPN1 | | | GSSG | HSA |
| | | | | | (1-398); | | | GSSG | |
| | | | | | K60G; | | | GSSG | |
| | | | | | V62I; | | | (SEQ | |
| | | | | | V180I; | | | ID | |
| | | | | | S192M; | | | NO: | |
| | | | | | M272L; | | | 33) | |
| | | | | | N276I; | | | | |
| | | | | | E284I; | | | | |
| 252 | | | | | hCPN1 | | | GSSG | HSA |
| | | | | | (1-398); | | | GSSG | |
| | | | | | K60M; | | | GSSG | |
| | | | | | V62I; | | | (SEQ | |
| | | | | | V180I; | | | ID | |
| | | | | | M184F; | | | NO: | |
| | | | | | S192A; | | | 33) | |
| | | | | | M272L; | | | | |
| | | | | | N276L; | | | | |
| | | | | | E284I; | | | | |
| 253 | | | | | hCPN1 | | | GSSG | HSA |
| | | | | | (1-398); | | | GSSG | |
| | | | | | K60F; | | | GSSG | |
| | | | | | V180Y; | | | (SEQ | |
| | | | | | S192A; | | | ID | |
| | | | | | N276T; | | | NO: | |
| | | | | | E284L; | | | 33) | |
| 254 | | | | | hCPN1 | | | GSSG | HSA |
| | | | | | (1-398); | | | GSSG | |
| | | | | | K60F; | | | GSSG | |
| | | | | | V62A; | | | (SEQ | |
| | | | | | V180Y; | | | ID | |
| | | | | | M184L; | | | NO: | |
| | | | | | S192A; | | | 33) | |
| | | | | | M272A; | | | | |
| | | | | | N276T; | | | | |
| | | | | | E284I; | | | | |
| 255 | | | | | hCPN1 | | | GSSG | HSA |
| | | | | | (1-398); | | | GSSG | |
| | | | | | K60F; | | | GSSG | |
| | | | | | V180Y; | | | (SEQ | |
| | | | | | S192A; | | | ID | |
| | | | | | N276T; | | | NO: | |
| | | | | | E284I; | | | 33) | |
| 256 | | | | | hCPN1 | | | GSSG | HSA |
| | | | | | (1-398); | | | GSSG | |
| | | | | | K60Y; | | | GSSG | |
| | | | | | V180Y; | | | (SEQ | |
| | | | | | M184L; | | | ID | |
| | | | | | S192A; | | | NO: | |
| | | | | | M272I; | | | 33) | |
| | | | | | N276T; | | | | |
| | | | | | E284I; | | | | |
| 257 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | N276V; | | | NO: | |
| | | | | | C282T; | | | 33) | |
| | | | | | E284I; | | | | |
| 258 | | | | | hCPN1 | | | GSSG | HSA |
| | | | | | (1-398); | | | GSSG | |
| | | | | | K60A; | | | GSSG | |
| | | | | | V62L; | | | (SEQ | |
| | | | | | M184L; | | | ID | |
| | | | | | S192M; | | | NO: | |
| | | | | | M272L; | | | 33) | |
| | | | | | N276I; | | | | |
| | | | | | C282T; | | | | |
| | | | | | E284V; | | | | |
| 259 | | | | | hCPN1 | | | GSSG | HSA |
| | | | | | (1-398); | | | GSSG | |
| | | | | | K60F; | | | GSSG | |
| | | | | | V62I; | | | (SEQ | |
| | | | | | V180I; | | | ID | |
| | | | | | M184L; | | | NO: | |
| | | | | | S192A; | | | 33) | |
| | | | | | M272L; | | | | |
| | | | | | N276I; | | | | |
| | | | | | C282T; | | | | |
| | | | | | E284I; | | | | |
| 260 | | | | | hCPN1 | | | GSSG | HSA |
| | | | | | (1-398); | | | GSSG | |
| | | | | | K60G; | | | GSSG | |
| | | | | | V180F; | | | (SEQ | |
| | | | | | S192M; | | | ID | |
| | | | | | N276M; | | | NO: | |
| | | | | | C282T; | | | 33) | |
| | | | | | E284V; | | | | |
| 261 | | | | | hCPN1 | | | GSSG | HSA |
| | | | | | (1-398); | | | GSSG | |
| | | | | | K60G; | | | GSSG | |
| | | | | | V62I; | | | (SEQ | |
| | | | | | V180I; | | | ID | |
| | | | | | M184Q; | | | NO: | |
| | | | | | S192Q; | | | 33) | |
| | | | | | N276I; | | | | |
| | | | | | C282S; | | | | |
| | | | | | E284I; | | | | |
| 262 | | | | | hCPN1 | | | GSSG | HSA |
| | | | | | (1-398); | | | GSSG | |
| | | | | | K60F; | | | GSSG | |
| | | | | | S192A; | | | (SEQ | |
| | | | | | N276W; | | | ID | |
| | | | | | E284V; | | | NO: | |
| | | | | | | | | 33) | |
| 263 | | | | | hCPN1 | | | GSSG | HSA |
| | | | | | (1-398); | | | GSSG | |
| | | | | | K60G; | | | GSSG | |
| | | | | | S192M; | | | (SEQ | |
| | | | | | N276W; | | | ID | |
| | | | | | E284V; | | | NO: | |
| | | | | | | | | 33) | |
| 264 | | | | | hCPN1 | | | GSSG | HSA |
| | | | | | (1-398); | | | GSSG | |
| | | | | | K60A; | | | GSSG | |
| | | | | | S192M; | | | (SEQ | |
| | | | | | N276I; | | | ID | |
| | | | | | E284V; | | | NO: | |
| | | | | | | | | 33) | |
| 265 | | | | | hCPN1 | | | GSSG | HSA |
| | | | | | (1-398); | | | GSSG | |
| | | | | | K60F; | | | GSSG | |
| | | | | | S192A; | | | (SEQ | |
| | | | | | N276W; | | | ID | |
| | | | | | C282T; | | | NO: | |
| | | | | | E284V; | | | 33) | |
| 266 | | | | | hCPN1 | | | GSSG | HSA |
| | | | | | (1-398); | | | GSSG | |
| | | | | | K60G; | | | GSSG | |
| | | | | | S192A; | | | (SEQ | |
| | | | | | N276W; | | | ID | |
| | | | | | C282T; | | | NO: | |
| | | | | | E284V; | | | 33) | |
| 267 | | | | | hCPN1 | | | GSSG | HSA |
| | | | | | (1-398); | | | GSSG | |
| | | | | | K60F; | | | GSSG | |
| | | | | | S192A; | | | (SEQ | |
| | | | | | N276W; | | | ID | |
| | | | | | C282L; | | | NO: | |
| | | | | | E284V; | | | 33) | |
| 268 | | | | | hCPN1 | | | GSSG | HSA |
| | | | | | (1-398); | | | GSSG | |
| | | | | | Y266F; | | | GSSG | |
| | | | | | E288Q; | | | (SEQ | |
| | | | | | | | | ID | |
| | | | | | | | | NO: | |
| | | | | | | | | 33) | |
| 269 | | | | | hCPN1 | | TEV | GSSG | HSA |
| | | | | | (1-398); | | | GSSG | |
| | | | | | Y266F; | | | GSSG | |
| | | | | | E288Q; | | | (SEQ | |
| | | | | | | | | ID | |
| | | | | | | | | NO: | |
| | | | | | | | | 33) | |
| 270 | | | | | hCPN1 | | | GSSG | HSA |
| | | | | | (1-398); | | | GSSG | |
| | | | | | W244A; | | | GSSG | |
| | | | | | | | | (SEQ | |
| | | | | | | | | ID | |
| | | | | | | | | NO: | |
| | | | | | | | | 33) | |
| 271 | | | | | hCPN1 | | | GSSG | HSA |
| | | | | | (1-398); | | | GSSG | |
| | | | | | Q247A; | | | GSSG | |
| | | | | | | | | (SEQ | |
| | | | | | | | | ID | |
| | | | | | | | | NO: | |
| | | | | | | | | 33) | |
| 272 | | | | | hCPN1 | | | GSSG | HSA |
| | | | | | (1-398); | | | GSSG | |
| | | | | | W249A; | | | GSSG | |
| | | | | | | | | (SEQ | |
| | | | | | | | | ID | |
| | | | | | | | | NO: | |
| | | | | | | | | 33) | |
| 273 | | | | | hCPN1 | | | GSSG | HSA |
| | | | | | (1-398); | | | GSSG | |
| | | | | | D253A; | | | GSSG | |
| | | | | | | | | (SEQ | |
| | | | | | | | | ID | |
| | | | | | | | | NO: | |
| | | | | | | | | 33) | |
| 274 | | | | | hCPN1 | | | GSSG | HSA |
| | | | | | (1-398); | | | GSSG | |
| | | | | | Y254A; | | | GSSG | |
| | | | | | | | | (SEQ | |
| | | | | | | | | ID | |
| | | | | | | | | NO: | |
| | | | | | | | | 33) | |
| 275 | | | | | hCPN1 | | | GSSG | HSA |
| | | | | | (1-398); | | | GSSG | |
| | | | | | D292A; | | | GSSG | |
| | | | | | | | | (SEQ | |
| | | | | | | | | ID | |
| | | | | | | | | NO: | |
| | | | | | | | | 33) | |
| 276 | | | | | hCPN1 | | | GSSG | HSA |
| | | | | | (1-398); | | | GSSG | |
| | | | | | E298A; | | | GSSG | |
| | | | | | | | | (SEQ | |
| | | | | | | | | ID | |
| | | | | | | | | NO: | |
| | | | | | | | | 33) | |
| 277 | | | | | hCPN1 | | | GSSG | HSA |
| | | | | | (1-398); | | | GSSG | |
| | | | | | E299A; | | | GSSG | |
| | | | | | | | | (SEQ | |
| | | | | | | | | ID | |
| | | | | | | | | NO: | |
| | | | | | | | | 33) | |
| 278 | | | | | hCPN1 | | | GSSG | HSA |
| | | | | | (1-398); | | | GSSG | |
| | | | | | R302A; | | | GSSG | |
| | | | | | | | | (SEQ | |
| | | | | | | | | ID | |
| | | | | | | | | NO: | |
| | | | | | | | | 33) | |
| 279 | | | | | hCPN1 | | | GSSG | HSA |
| | | | | | (1-398); | | | GSSG | |
| | | | | | K293A; | | | GSSG | |
| | | | | | | | | (SEQ | |
| | | | | | | | | ID | |
| | | | | | | | | NO: | |
| | | | | | | | | 33) | |
| 280 | | | | | hCPN1 | | | GSSG | HSA |
| | | | | | (1-398); | | | GSSG | |
| | | | | | P296A; | | | GSSG | |
| | | | | | | | | (SEQ | |
| | | | | | | | | ID | |
| | | | | | | | | NO: | |
| | | | | | | | | 33) | |
| 281 | | | | | hCPN1 | | | GSSG | HSA |
| | | | | | (1-398); | | | GSSG | |
| | | | | | G126A; | | | GSSG | |
| | | | | | | | | (SEQ | |
| | | | | | | | | ID | |
| | | | | | | | | NO: | |
| | | | | | | | | 33) | |
| 282 | | | | | hCPN1 | | | GSSG | HSA |
| | | | | | (1-398); | | | GSSG | |
| | | | | | Y127A; | | | GSSG | |
| | | | | | | | | (SEQ | |
| | | | | | | | | ID | |
| | | | | | | | | NO: | |
| | | | | | | | | 33) | |
| 283 | | | | | hCPN1 | | | GSSG | HSA |
| | | | | | (1-398); | | | GSSG | |
| | | | | | L128A; | | | GSSG | |
| | | | | | | | | (SEQ | |
| | | | | | | | | ID | |
| | | | | | | | | NO: | |
| | | | | | | | | 33) | |
| 284 | | | | | hCPN1 | | | GSSG | HSA |
| | | | | | (1-398); | | | GSSG | |
| | | | | | Q247L; | | | GSSG | |
| | | | | | | | | (SEQ | |
| | | | | | | | | ID | |
| | | | | | | | | NO: | |
| | | | | | | | | 33) | |
| 285 | | | | | hCPN1 | | | GSSG | HSA |
| | | | | | (1-398); | | | GSSG | |
| | | | | | W244K; | | | GSSG | |
| | | | | | Q247L; | | | (SEQ | |
| | | | | | | | | ID | |
| | | | | | | | | NO: | |
| | | | | | | | | 33) | |
| 286 | | | | | hCPN1 | | | GSSG | HSA |
| | | | | | (1-398); | | | GSSG | |
| | | | | | W244L; | | | GSSG | |
| | | | | | Q247M; | | | | |
| | | | | | W249Y; | | | (SEQ | |
| | | | | | | | | ID | |
| | | | | | | | | NO: | |
| | | | | | | | | 33) | |
| 287 | | | | | hCPN1 | | | GSSG | HSA |
| | | | | | (1-398); | | | GSSG | |
| | | | | | W244L; | | | GSSG | |
| | | | | | Q247L; | | | (SEQ | |
| | | | | | W249Y; | | | ID | |
| | | | | | | | | NO: | |
| | | | | | | | | 33) | |
| 288 | | | | | hCPN1 | | | GSSG | HSA |
| | | | | | (1-398); | | | GSSG | |
| | | | | | W244F; | | | GSSG | |
| | | | | | Q247T; | | | (SEQ | |
| | | | | | | | | ID | |
| | | | | | | | | NO: | |
| | | | | | | | | 33 | |
| 289 | | | | | hCPN1 | | | GSSG | HSA |
| | | | | | (1-398); | | | GSSG | |
| | | | | | Q247M; | | | GSSG | |
| | | | | | | | | (SEQ | |
| | | | | | | | | ID | |
| | | | | | | | | NO: | |
| | | | | | | | | 33) | |
| 290 | | | | | hCPN1 | | | GSSG | HSA |
| | | | | | (1-398); | | | GSSG | |
| | | | | | W249Y; | | | GSSG | |
| | | | | | | | | (SEQ | |
| | | | | | | | | ID | |
| | | | | | | | | NO: | |
| | | | | | | | | 33) | |
| 291 | | | | | hCPN1 | | | GSSG | HSA |
| | | | | | (1-398); | | | GSSG | |
| | | | | | W244D; | | | GSSG | |
| | | | | | Q247L; | | | (SEQ | |
| | | | | | | | | ID | |
| | | | | | | | | NO: | |
| | | | | | | | | 33) | |
| 292 | | | | | hCPN1 | | | GSSG | HSA |
| | | | | | (1-398); | | | GSSG | |
| | | | | | Y254R; | | | GSSG | |
| | | | | | | | | (SEQ | |
| | | | | | | | | ID | |
| | | | | | | | | NO: | |
| | | | | | | | | 33) | |
| 293 | | | | | hCPN1 | | | GSSG | HSA |
| | | | | | (1-398); | | | GSSG | |
| | | | | | Q247L; | | | GSSG | |
| | | | | | Y254R; | | | (SEQ | |
| | | | | | | | | ID | |
| | | | | | | | | NO: | |
| | | | | | | | | 33) | |
| 294 | | | | | hCPN1 | | | GSSG | HSA |
| | | | | | (1-398); | | | GSSG | |
| | | | | | W244K; | | | GSSG | |
| | | | | | Q247L; | | | (SEQ | |
| | | | | | D253T; | | | ID | |
| | | | | | | | | NO: | |
| | | | | | | | | 33) | |
| 295 | | | | | hCPN1 | | | GSSG | HSA |
| | | | | | (1-398); | | | GSSG | |
| | | | | | Q247L; | | | GSSG | |
| | | | | | D253T; | | | (SEQ | |
| | | | | | | | | ID | |
| | | | | | | | | NO: | |
| | | | | | | | | 33) | |
| 296 | | | | | hCPN1 | | | GSSG | HSA |
| | | | | | (1-398); | | | GSSG | |
| | | | | | D292L; | | | GSSG | |
| | | | | | | | | (SEQ | |
| | | | | | | | | ID | |
| | | | | | | | | NO: | |
| | | | | | | | | 33) | |
| 297 | | | | | hCPN1 | | | GSSG | HSA |
| | | | | | (1-398); | | | GSSG | |
| | | | | | R302M; | | | GSSG | |
| | | | | | | | | (SEQ | |
| | | | | | | | | ID | |
| | | | | | | | | NO: | |
| | | | | | | | | 33) | |
| 298 | | | | | hCPN1 | | | GSSG | HSA |
| | | | | | (1-398); | | | GSSG | |
| | | | | | R302W; | | | GSSG | |
| | | | | | | | | (SEQ | |
| | | | | | | | | ID | |
| | | | | | | | | NO: | |
| | | | | | | | | 33) | |
| 299 | | | | | hCPN1 | | | GSSG | HSA |
| | | | | | (1-398); | | | GSSG | |
| | | | | | R.302K; | | | GSSG | |
| | | | | | | | | (SEQ | |
| | | | | | | | | ID | |
| | | | | | | | | NO: | |
| | | | | | | | | 33) | |
| 300 | | | | | hCPN1 | | | GSSG | HSA |
| | | | | | (1-398); | | | GSSG | |
| | | | | | R302L; | | | GSSG | |
| | | | | | | | | (SEQ | |
| | | | | | | | | ID | |
| | | | | | | | | NO: | |
| | | | | | | | | 33) | |
| 301 | | | | | hCPN1 | | | GSSG | HSA |
| | | | | | (1-398); | | | GSSG | |
| | | | | | R302Q; | | | GSSG | |
| | | | | | | | | (SEQ | |
| | | | | | | | | ID | |
| | | | | | | | | NO: | |
| | | | | | | | | 33) | |
| 302 | | | | | hCPN1 | | | GSSG | HSA |
| | | | | | (1-398); | | | GSSG | |
| | | | | | K293P; | | | GSSG | |
| | | | | | R302M; | | | (SEQ | |
| | | | | | | | | ID | |
| | | | | | | | | NO: | |
| | | | | | | | | 33) | |
| 303 | | | | | hCPN1 | | | GSSG | HSA |
| | | | | | (1-398); | | | GSSG | |
| | | | | | D292L; | | | GSSG | |
| | | | | | K293A; | | | (SEQ | |
| | | | | | | | | ID | |
| | | | | | | | | NO: | |
| | | | | | | | | 33) | |
| 304 | | | | | hCPN1 | | | GSSG | HSA |
| | | | | | (1-398); | | | GSSG | |
| | | | | | K293Y; | | | GSSG | |
| | | | | | | | | (SEQ | |
| | | | | | | | | ID | |
| | | | | | | | | NO: | |
| | | | | | | | | 33) | |
| 305 | | | | | hCPN1 | | | GSSG | HSA |
| | | | | | (1-398); | | | GSSG | |
| | | | | | K293Y; | | | GSSG | |
| | | | | | P296D; | | | (SEQ | |
| | | | | | | | | ID | |
| | | | | | | | | NO: | |
| | | | | | | | | 33) | |
| 306 | | | | | hCPN1 | | | GSSG | HSA |
| | | | | | (1-398); | | | GSSG | |
| | | | | | K293P; | | | GSSG | |
| | | | | | R302I; | | | (SEQ | |
| | | | | | | | | ID | |
| | | | | | | | | NO: | |
| | | | | | | | | 33) | |
| 307 | | | | | hCPN1 | | | GSSG | HSA |
| | | | | | (1-398); | | | GSSG | |
| | | | | | K293M; | | | GSSG | |
| | | | | | P296D; | | | (SEQ | |
| | | | | | | | | ID | |
| | | | | | | | | NO: | |
| | | | | | | | | 33) | |
| 308 | | | | | hCPN1 | | | GSSG | HSA |
| | | | | | (1-398); | | | GSSG | |
| | | | | | Y127L; | | | GSSG | |
| | | | | | K293P; | | | (SEQ | |
| | | | | | | | | ID | |
| | | | | | | | | NO: | |
| | | | | | | | | 33) | |
| 309 | | | | | hCPN1 | | | GSSG | HSA |
| | | | | | (1-398); | | | GSSG | |
| | | | | | Y127A; | | | GSSG | |
| | | | | | K293Y; | | | (SEQ | |
| | | | | | | | | ID | |
| | | | | | | | | NO: | |
| | | | | | | | | 33) | |
| 310 | | | | | hCPN1 | | | GSSG | HSA |
| | | | | | (1-398); | | | GSSG | |
| | | | | | D292L; | | | GSSG | |
| | | | | | K293A; | | | (SEQ | |
| | | | | | | | | ID | |
| | | | | | | | | NO: | |
| | | | | | | | | 33) | |
| 311 | | | | | hCPN1 | | | GSSG | HSA |
| | | | | | (1-398); | | | GSSG | |
| | | | | | Y127A; | | | GSSG | |
| | | | | | K293M; | | | (SEQ | |
| | | | | | | | | ID | |
| | | | | | | | | NO: | |
| | | | | | | | | 33) | |
| 312 | | | | | hCPN1 | | | GSSG | HSA |
| | | | | | (1-398); | | | GSSG | |
| | | | | | Y127K; | | | GSSG | |
| | | | | | K293P; | | | | |
| | | | | | | | | (SEQ | |
| | | | | | | | | ID | |
| | | | | | | | | NO: | |
| | | | | | | | | 33) | |
| 313 | | | | | hCPN1 | | | GSSG | HSA |
| | | | | | (1-398); | | | GSSG | |
| | | | | | D292V; | | | GSSG | |
| | | | | | K293A; | | | (SEQ | |
| | | | | | | | | ID | |
| | | | | | | | | NO: | |
| | | | | | | | | 33) | |
| 314 | | | | | hCPN1 | | | GSSG | HSA |
| | | | | | (1-398); | | | GSSG | |
| | | | | | K293P; | | | GSSG | |
| | | | | | | | | (SEQ | |
| | | | | | | | | ID | |
| | | | | | | | | NO: | |
| | | | | | | | | 33) | |
| 315 | | | | | hCPN1 | | | GSSG | HSA |
| | | | | | (1-398); | | | GSSG | |
| | | | | | Y127K; | | | GSSG | |
| | | | | | L128D; | | | (SEQ | |
| | | | | | K293Y; | | | ID | |
| | | | | | | | | NO: | |
| | | | | | | | | 33) | |
| 316 | | | | | hCPN1 | | | GSSG | HSA |
| | | | | | (1-398); | | | GSSG | |
| | | | | | Y127L; | | | GSSG | |
| | | | | | L128H; | | | (SEQ | |
| | | | | | K293P; | | | ID | |
| | | | | | | | | NO: | |
| | | | | | | | | 33) | |
| 317 | | | | | hCPN1 | | | GSSG | HSA |
| | | | | | (1-398); | | | GSSG | |
| | | | | | Y127K; | | | GSSG | |
| | | | | | L128H; | | | (SEQ | |
| | | | | | K293P; | | | ID | |
| | | | | | | | | NO: | |
| | | | | | | | | 33) | |
| 318 | | | | | hCPN1 | | | GSSG | HSA |
| | | | | | (1-398); | | | GSSG | |
| | | | | | L128D; | | | GSSG | |
| | | | | | | | | (SEQ | |
| | | | | | | | | ID | |
| | | | | | | | | NO: | |
| | | | | | | | | 33) | |
| 319 | | | | | hCPN1 | | | GSSG | HSA |
| | | | | | (1-398); | | | GSSG | |
| | | | | | C251S; | | | GSSG | |
| | | | | | C291S; | | | (SEQ | |
| | | | | | | | | ID | |
| | | | | | | | | NO: | |
| | | | | | | | | 33) | |
| 320 | | | | | hCPN1 | | | GSSG | HSA |
| | | | | | (1-398); | | | GSSG | |
| | | | | | T223C; | | | GSSG | |
| | | | | | A263C; | | | (SEQ | |
| | | | | | | | | ID | |
| | | | | | | | | NO: | |
| | | | | | | | | 33) | |
| 321 | | | | | hCPN1 | | | GSSG | HSA |
| | | | | | (1-398); | | | GSSG | |
| | | | | | S209C; | | | GSSG | |
| | | | | | A263C; | | | (SEQ | |
| | | | | | | | | ID | |
| | | | | | | | | NO: | |
| | | | | | | | | 33) | |
| 322 | | | | | hCPN1 | | | GSSG | HSA |
| | | | | | (1-398); | | | GSSG | |
| | | | | | S209C; | | | GSSG | |
| | | | | | S267C; | | | (SEQ | |
| | | | | | | | | ID | |
| | | | | | | | | NO: | |
| | | | | | | | | 33) | |
| 323 | | | | | hCPN1 | | | GSSG | HSA |
| | | | | | (1-398); | | | GSSG | |
| | | | | | S267C; | | | GSSG | |
| | | | | | S269C; | | | (SEQ | |
| | | | | | | | | ID | |
| | | | | | | | | NO: | |
| | | | | | | | | 33) | |
| 324 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | T223C; | | | NO: | |
| | | | | | A263C; | | | 33) | |
| | | | | | N276V; | | | | |
| | | | | | E284I; | | | | |
| 325 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | S209C; | | | NO: | |
| | | | | | A263C; | | | 33) | |
| | | | | | N276V; | | | | |
| | | | | | E284I; | | | | |
| 326 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | S209C; | | | NO: | |
| | | | | | S267C; | | | 33) | |
| | | | | | N276V; | | | | |
| | | | | | E284I; | | | | |
| 327 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | S267C; | | | NO: | |
| | | | | | S269C; | | | 33) | |
| | | | | | N276V; | | | | |
| | | | | | E284I; | | | | |
| 328 | | | | | hCPN1 | | | GSSG | HSA |
| | | | | | (1-398); | | | GSSG | |
| | | | | | G121A; | | | GSSG | |
| | | | | | | | | (SEQ | |
| | | | | | | | | ID | |
| | | | | | | | | NO: | |
| | | | | | | | | 33) | |
| 329 | | | | | hCPN1 | | | GSSG | HSA |
| | | | | | (1-398); | | | GSSG | |
| | | | | | P122A; | | | GSSG | |
| | | | | | | | | (SEQ | |
| | | | | | | | | ID | |
| | | | | | | | | NO: | |
| | | | | | | | | 33) | |
| 330 | | | | | hCPN1 | | | GSSG | HSA |
| | | | | | (1-398); | | | GSSG | |
| | | | | | N123A; | | | GSSG | |
| | | | | | | | | (SEQ | |
| | | | | | | | | ID | |
| | | | | | | | | NO: | |
| | | | | | | | | 33) | |
| 331 | | | | | hCPN1 | | | GSSG | HSA |
| | | | | | (1-398); | | | GSSG | |
| | | | | | K124A; | | | GSSG | |
| | | | | | | | | (SEQ | |
| | | | | | | | | ID | |
| | | | | | | | | NO: | |
| | | | | | | | | 33) | |
| 332 | | | | | hCPN1 | | | GSSG | HSA |
| | | | | | (1-398); | | | GSSG | |
| | | | | | P125A; | | | GSSG | |
| | | | | | | | | (SEQ | |
| | | | | | | | | ID | |
| | | | | | | | | NO: | |
| | | | | | | | | 33) | |
| 333 | | | | | hCPN1 | | | GSSG | HSA |
| | | | | | (1-398); | | | GSSG | |
| | | | | | D138A; | | | GSSG | |
| | | | | | | | | (SEQ | |
| | | | | | | | | ID | |
| | | | | | | | | NO: | |
| | | | | | | | | 33) | |
| 334 | | | | | hCPN1 | | | GSSG | HSA |
| | | | | | (1-398); | | | GSSG | |
| | | | | | V180A; | | | GSSG | |
| | | | | | | | | (SEQ | |
| | | | | | | | | ID | |
| | | | | | | | | NO: | |
| | | | | | | | | 33) | |
| 335 | | | | | hCPN1 | | | GSSG | HSA |
| | | | | | (1-398); | | | GSSG | |
| | | | | | F189A; | | | GSSG | |
| | | | | | | | | (SEQ | |
| | | | | | | | | ID | |
| | | | | | | | | NO: | |
| | | | | | | | | 33) | |
| 336 | | | | | hCPN1 | | | GSSG | HSA |
| | | | | | (1-398); | | | GSSG | |
| | | | | | A199A; | | | GSSG | |
| | | | | | | | | (SEQ | |
| | | | | | | | | ID | |
| | | | | | | | | NO: | |
| | | | | | | | | 33) | |
| 337 | | | | | hCPN1 | | | GSSG | HSA |
| | | | | | (1-398); | | | GSSG | |
| | | | | | N276A; | | | GSSG | |
| | | | | | | | | (SEQ | |
| | | | | | | | | ID | |
| | | | | | | | | NO: | |
| | | | | | | | | 33) | |
| 338 | | | | | hCPN1 | | | GSSG | HSA |
| | | | | | (1-398); | | | GSSG | |
| | | | | | E284A; | | | GSSG | |
| | | | | | | | | (SEQ | |
| | | | | | | | | ID | |
| | | | | | | | | NO: | |
| | | | | | | | | 33) | |
| 339 | | | | | hCPN1 | | | GSSG | HSA |
| | | | | | (1-398); | | | GSSG | |
| | | | | | E297A; | | | GSSG | |
| | | | | | | | | (SEQ | |
| | | | | | | | | ID | |
| | | | | | | | | NO: | |
| | | | | | | | | 33) | |
| 340 | | | | | hCPN1 | | | GSSG | HSA |
| | | | | | (1-398); | | | GSSG | |
| | | | | | L300A; | | | GSSG | |
| | | | | | | | | (SEQ | |
| | | | | | | | | ID | |
| | | | | | | | | NO: | |
| | | | | | | | | 33) | |
| 341 | | | | | hCPN1 | | | GSSG | HSA |
| | | | | | (1-398); | | | GSSG | |
| | | | | | Q301A; | | | GSSG | |
| | | | | | | | | (SEQ | |
| | | | | | | | | ID | |
| | | | | | | | | NO: | |
| | | | | | | | | 33) | |
| 342 | | | | | hCPN1 | | | GSSG | HSA |
| | | | | | (1-398); | | | GSSG | |
| | | | | | K60A; | | | GSSG | |
| | | | | | | | | (SEQ | |
| | | | | | | | | ID | |
| | | | | | | | | NO: | |
| | | | | | | | | 33) | |
| 343 | | | | | hCPN1 | | | GSSG | HSA |
| | | | | | (1-398); | | | GSSG | |
| | | | | | H66A; | | | GSSG | |
| | | | | | | | | (SEQ | |
| | | | | | | | | ID | |
| | | | | | | | | NO: | |
| | | | | | | | | 33) | |
| 344 | | | | | hCPN1 | | | GSSG | HSA |
| | | | | | (1-398); | | | GSSG | |
| | | | | | Q78A; | | | GSSG | |
| | | | | | | | | (SEQ | |
| | | | | | | | | ID | |
| | | | | | | | | NO: | |
| | | | | | | | | 33) | |
| 345 | | | | | hCPN1 | | | GSSG | HSA |
| | | | | | (1-398); | | | GSSG | |
| | | | | | K60F; | | | GSSG | |
| | | | | | V180F; | | | (SEQ | |
| | | | | | S192A; | | | ID | |
| | | | | | N276V; | | | NO: | |
| | | | | | E284I; | | | 33) | |
| 346 | | | | | hCPN1 | | | GSSG | HSA |
| | | | | | (1-398); | | | GSSG | |
| | | | | | K60F; | | | GSSG | |
| | | | | | A70V; | | | (SEQ | |
| | | | | | V180F; | | | ID | |
| | | | | | | | | NO: | |
| | | | | | | | | 33) | |
| | | | | | S192A; | | | | |
| | | | | | N276V; | | | | |
| | | | | | E284I | | | | |
| 347 | | | | | hCPN1 | | | GSSG | HSA |
| | | | | | (1-398); | | | GSSG | |
| | | | | | K60F; | | | GSSG | |
| | | | | | N68D; | | | (SEQ | |
| | | | | | P125S; | | | ID | |
| | | | | | V180F; | | | NO: | |
| | | | | | S192A; | | | 33) | |
| | | | | | N276V; | | | | |
| | | | | | E284I | | | | |
| 348 | | | | | hCPN1 | | | GSSG | HSA |
| | | | | | (1-398); | | | GSSG | |
| | | | | | K60F; | | | GSSG | |
| | | | | | N123D; | | | (SEQ | |
| | | | | | K124D; | | | ID | |
| | | | | | P125S; | | | NO: | |
| | | | | | G126Q; | | | 33) | |
| | | | | | V180F; | | | | |
| | | | | | S192A; | | | | |
| | | | | | N276V; | | | | |
| | | | | | E284I | | | | |
| 349 | | | | | hCPN1 | | | GSSG | HSA |
| | | | | | (1-398); | | | GSSG | |
| | | | | | K60F; | | | GSSG | |
| | | | | | G121T; | | | (SEQ | |
| | | | | | P122Y; | | | ID | |
| | | | | | K124Q; | | | NO: | |
| | | | | | P125D; | | | 33) | |
| | | | | | V180F; | | | | |
| | | | | | S192A; | | | | |
| | | | | | N276V; | | | | |
| | | | | | E284I | | | | |
| 350 | | | | | hCPN1 | | | GSSG | HSA |
| | | | | | (1-398); | | | GSSG | |
| | | | | | K60F; | | | GSSG | |
| | | | | | N68D; | | | (SEQ | |
| | | | | | A70V; | | | ID | |
| | | | | | P125S; | | | NO: | |
| | | | | | V180F; | | | 33) | |
| | | | | | S192A; | | | | |
| | | | | | N276V; | | | | |
| | | | | | E284I | | | | |
| 351 | | | | | hCPN1 | | | GSSG | HSA |
| | | | | | (1-398) | | | GSSG | |
| | | | | | | | | (SEQ | |
| | | | | | | | | ID | |
| | | | | | | | | NO: | |
| | | | | | | | | 42) | |
| 352 | | | | | hCPN1 | | | GSSG | HSA |
| | | | | | (1-398); | | | GSSG | |
| | | | | | W244Y; | | | GSSG | |
| | | | | | Q247S; | | | (SEQ | |
| | | | | | | | | ID | |
| | | | | | | | | NO: | |
| | | | | | | | | 33) | |
| 353 | | | | | hCPN1 | | | GSSG | HSA |
| | | | | | (1-398); | | | GSSG | |
| | | | | | W244Y; | | | GSSG | |
| | | | | | Q247L; | | | (SEQ | |
| | | | | | W249L; | | | ID | |
| | | | | | | | | NO: | |
| | | | | | | | | 33) | |
| 354 | | | | | hCPN1 | | | GSSG | HSA |
| | | | | | (1-398); | | | GSSG | |
| | | | | | W244Y; | | | GSSG | |
| | | | | | Q247L; | | | (SEQ | |
| | | | | | W249M; | | | ID | |
| | | | | | | | | NO: | |
| | | | | | | | | 33) | |
| 355 | | | | | hCPN1 | | | GSSG | HSA |
| | | | | | (1-398); | | | GSSG | |
| | | | | | Q247Y; | | | GSSG | |
| | | | | | W249M; | | | (SEQ | |
| | | | | | | | | ID | |
| | | | | | | | | NO: | |
| | | | | | | | | 33) | |
| 356 | | | | | hCPN1 | | | GSSG | HSA |
| | | | | | (1-398); | | | GSSG | |
| | | | | | Q247Y; | | | GSSG | |
| | | | | | | | | (SEQ | |
| | | | | | | | | ID | |
| | | | | | | | | NO: | |
| | | | | | | | | 33) | |
| 357 | | | | | hCPN1 | | | GSSG | HSA |
| | | | | | (1-398); | | | GSSG | |
| | | | | | W244Y; | | | GSSG | |
| | | | | | Q247L; | | | (SEQ | |
| | | | | | W249F; | | | ID | |
| | | | | | | | | NO: | |
| | | | | | | | | 33) | |
| 358 | | | | | hCPN1 | | | GSSG | HSA |
| | | | | | (1-398); | | | GSSG | |
| | | | | | W244Y; | | | GSSG | |
| | | | | | Q247L; | | | (SEQ | |
| | | | | | W249Y; | | | ID | |
| | | | | | | | | NO: | |
| | | | | | | | | 33) | |
| 359 | | | | | hCPN1 | | | GSSG | HSA |
| | | | | | (1-398); | | | GSSG | |
| | | | | | Q247Y; | | | GSSG | |
| | | | | | W249L; | | | (SEQ | |
| | | | | | | | | ID | |
| | | | | | | | | NO: | |
| | | | | | | | | 33) | |
| 360 | | | | | hCPN1 | | | GSSG | HSA |
| | | | | | (1-398); | | | GSSG | |
| | | | | | W244Y; | | | GSSG | |
| | | | | | Q247M; | | | (SEQ | |
| | | | | | W249L; | | | ID | |
| | | | | | | | | NO: | |
| | | | | | | | | 33) | |
| 361 | | | | | hCPN1 | | | GSSG | HSA |
| | | | | | (1-398); | | | GSSG | |
| | | | | | W244D; | | | GSSG | |
| | | | | | Q247F; | | | (SEQ | |
| | | | | | W249H; | | | ID | |
| | | | | | | | | NO: | |
| | | | | | | | | 33) | |
| 362 | | | | | hCPN1 | | | GSSG | HSA |
| | | | | | (1-398); | | | GSSG | |
| | | | | | W244Y; | | | GSSG | |
| | | | | | Q247N; | | | (SEQ | |
| | | | | | W249F; | | | ID | |
| | | | | | | | | NO: | |
| | | | | | | | | 33) | |
| 363 | | | | | hCPN1 | | | GSSG | HSA |
| | | | | | (1-398); | | | GSSG | |
| | | | | | G243E; | | | GSSG | |
| | | | | | W244Y; | | | (SEQ | |
| | | | | | F246Y; | | | ID | |
| | | | | | Q247L; | | | NO: | |
| | | | | | W249Y; | | | 33) | |
| | | | | | N250S; | | | | |
| 364 | | | | | hCPN1 | | | GSSG | HSA |
| | | | | | (1-398); | | | GSSG | |
| | | | | | G243A; | | | GSSG | |
| | | | | | W244Y; | | | (SEQ | |
| | | | | | F246W; | | | ID | |
| | | | | | Q247S; | | | NO: | |
| | | | | | D253L; | | | 33) | |
| 365 | | | | | hCPN1 | | | GSSG | HSA |
| | | | | | (1-398); | | | GSSG | |
| | | | | | G243Y; | | | GSSG | |
| | | | | | W244M; | | | (SEQ | |
| | | | | | F246W; | | | ID | |
| | | | | | Q247S; | | | NO: | |
| | | | | | D253L; | | | 33) | |
| 366 | | | | | hCPN1 | | | GSSG | HSA |
| | | | | | (1-398); | | | GSSG | |
| | | | | | G243A; | | | GSSG | |
| | | | | | W244Y; | | | (SEQ | |
| | | | | | F246W; | | | ID | |
| | | | | | Q247A; | | | NO: | |
| | | | | | D253L; | | | 33) | |
| 367 | | | | | hCPN1 | | | GSSG | HSA |
| | | | | | (1-398); | | | GSSG | |
| | | | | | G243W; | | | GSSG | |
| | | | | | F246Y; | | | (SEQ | |
| | | | | | Q247L; | | | ID | |
| | | | | | N250S; | | | NO: | |
| | | | | | D253N; | | | 33) | |
| 368 | | | | | hCPN1 | | | GSSG | HSA |
| | | | | | (1-398); | | | GSSG | |
| | | | | | G243E; | | | GSSG | |
| | | | | | W244Y; | | | (SEQ | |
| | | | | | F246A; | | | ID | |
| | | | | | W249Y; | | | NO: | |
| | | | | | N250S; | | | 33) | |
| 369 | | | | | hCPN1 | | | GSSG | HSA |
| | | | | | (1-398); | | | GSSG | |
| | | | | | G243Y; | | | GSSG | |
| | | | | | W244I; | | | (SEQ | |
| | | | | | F246W; | | | ID | |
| | | | | | Q247S; | | | NO: | |
| | | | | | D253L; | | | 33) | |
| 370 | | | | | hCPN1 | | | GSSG | HSA |
| | | | | | (1-398); | | | GSSG | |
| | | | | | G243W; | | | GSSG | |
| | | | | | Q247Y; | | | (SEQ | |
| | | | | | N250S; | | | ID | |
| | | | | | D253N; | | | NO: | |
| | | | | | | | | 33) | |
| 371 | | | | | hCPN1 | | | GSSG | HSA |
| | | | | | (1-398); | | | GSSG | |
| | | | | | G243W; | | | GSSG | |
| | | | | | F246W; | | | (SEQ | |
| | | | | | Q247L; | | | ID | |
| | | | | | N250S; | | | NO: | |
| | | | | | D253N; | | | 33) | |
| 372 | | | | | hCPN1 | | | GSSG | HSA |
| | | | | | (1-398); | | | GSSG | |
| | | | | | G243W; | | | GSSG | |
| | | | | | F246L; | | | (SEQ | |
| | | | | | Q247Y; | | | ID | |
| | | | | | W249L; | | | NO: | |
| | | | | | N250S; | | | 33) | |
| | | | | | D253N; | | | | |
| 373 | | | | | hCPN1 | | | GSSG | HSA |
| | | | | | (1-398); | | | GSSG | |
| | | | | | G243W; | | | GSSG | |
| | | | | | W244D; | | | (SEQ | |
| | | | | | F246R; | | | ID | |
| | | | | | Q247F; | | | NO: | |
| | | | | | W249L; | | | 33) | |
| | | | | | N250S; | | | | |
| | | | | | D253N; | | | | |
| 374 | | | | | hCPN1 | | | GSSG | HSA |
| | | | | | (1-398); | | | GSSG | |
| | | | | | G243W; | | | GSSG | |
| | | | | | Q247Y; | | | (SEQ | |
| | | | | | W249L; | | | ID | |
| | | | | | N250S; | | | NO: | |
| | | | | | D253N; | | | 33) | |
| 375 | | | | | hCPN1 | | | GSSG | HSA |
| | | | | | (1-398); | | | GSSG | |
| | | | | | G243W; | | | GSSG | |
| | | | | | F246R; | | | (SEQ | |
| | | | | | Q247F; | | | ID | |
| | | | | | W249L; | | | NO: | |
| | | | | | N250S; | | | 33) | |
| | | | | | D253N; | | | | |
| 376 | | | | | hCPN1 | | | GSSG | HSA |
| | | | | | (1-398); | | | GSSG | |
| | | | | | G243W; | | | GSSG | |
| | | | | | W244D; | | | (SEQ | |
| | | | | | F246W; | | | ID | |
| | | | | | Q247Y; | | | NO: | |
| | | | | | W249F; | | | 33) | |
| | | | | | N250S; | | | | |
| | | | | | D253N; | | | | |
| 377 | | | | | hCPN1 | | | GSSG | HSA |
| | | | | | (1-398); | | | GSSG | |
| | | | | | R302E; | | | GSSG | |
| | | | | | G306A; | | | (SEQ | |
| | | | | | | | | ID | |
| | | | | | | | | NO: | |
| | | | | | | | | 33) | |
| 378 | | | | | hCPN1 | | | GSSG | HSA |
| | | | | | (1-398); | | | GSSG | |
| | | | | | G306Q; | | | GSSG | |
| | | | | | | | | (SEQ | |
| | | | | | | | | ID | |
| | | | | | | | | NO: | |
| | | | | | | | | 33) | |
| 379 | | | | | hCPN1 | | | GSSG | HSA |
| | | | | | (1-398); | | | GSSG | |
| | | | | | R302E; | | | GSSG | |
| | | | | | G306T; | | | (SEQ | |
| | | | | | | | | ID | |
| | | | | | | | | NO: | |
| | | | | | | | | 33) | |
| 380 | | | | | hCPN1 | | | GSSG | HSA |
| | | | | | (1-398); | | | GSSG | |
| | | | | | D292I; | | | GSSG | |
| | | | | | R302L; | | | (SEQ | |
| | | | | | G306H; | | | ID | |
| | | | | | | | | NO: | |
| | | | | | | | | 33) | |
| 381 | | | | | hCPN1 | | | GSSG | HSA |
| | | | | | (1-398); | | | GSSG | |
| | | | | | G306N; | | | GSSG | |
| | | | | | | | | (SEQ | |
| | | | | | | | | ID | |
| | | | | | | | | NO: | |
| | | | | | | | | 33) | |
| 382 | | | | | hCPN1 | | | GSSG | HSA |
| | | | | | (1-398); | | | GSSG | |
| | | | | | D2921; | | | GSSG | |
| | | | | | R302Q; | | | (SEQ | |
| | | | | | G306D; | | | ID | |
| | | | | | | | | NO: | |
| | | | | | | | | 33) | |
| 383 | | | | | hCPN1 | | | GSSG | HSA |
| | | | | | (1-398); | | | GSSG | |
| | | | | | R302E; | | | GSSG | |
| | | | | | G306N; | | | (SEQ | |
| | | | | | | | | ID | |
| | | | | | | | | NO: | |
| | | | | | | | | 33) | |
| 384 | | | | | hCPN1 | | | GSSG | HSA |
| | | | | | (1-398); | | | GSSG | |
| | | | | | D292I; | | | GSSG | |
| | | | | | G306N; | | | (SEQ | |
| | | | | | | | | ID | |
| | | | | | | | | NO: | |
| | | | | | | | | 33) | |
| 385 | | | | | hCPN1 | | | GSSG | HSA |
| | | | | | (1-398); | | | GSSG | |
| | | | | | G306F; | | | GSSG | |
| | | | | | | | | (SEQ | |
| | | | | | | | | ID | |
| | | | | | | | | NO: | |
| | | | | | | | | 33) | |
| 386 | | | | | hCPN1 | | | GSSG | HSA |
| | | | | | (1-398); | | | GSSG | |
| | | | | | D292I; | | | GSSG | |
| | | | | | R302L; | | | (SEQ | |
| | | | | | G306W; | | | ID | |
| | | | | | | | | NO: | |
| | | | | | | | | 33) | |
| 387 | | | | | hCPN1 | | | GSSG | HSA |
| | | | | | (1-398); | | | GSSG | |
| | | | | | D292I; | | | GSSG | |
| | | | | | R302D; | | | (SEQ | |
| | | | | | G306H; | | | ID | |
| | | | | | | | | NO: | |
| | | | | | | | | 33) | |
| 388 | | | | | hCPN1 | | | GSSG | HSA |
| | | | | | (1-398); | | | GSSG | |
| | | | | | D292I; | | | GSSG | |
| | | | | | R302E; | | | (SEQ | |
| | | | | | G306H; | | | ID | |
| | | | | | | | | NO: | |
| | | | | | | | | 33) | |
| 389 | | | | | hCPN1 | | | GSSG | HSA |
| | | | | | (1-398); | | | GSSG | |
| | | | | | Y127E; | | | GSSG | |
| | | | | | L128T; | | | (SEQ | |
| | | | | | | | | ID | |
| | | | | | | | | NO: | |
| | | | | | | | | 33) | |
| 390 | | | | | hCPN1 | | | GSSG | HSA |
| | | | | | (1-398); | | | GSSG | |
| | | | | | Y127L; | | | GSSG | |
| | | | | | L128T; | | | (SEQ | |
| | | | | | | | | ID | |
| | | | | | | | | NO: | |
| | | | | | | | | 33) | |
| 391 | | | | | hCPN1 | | | GSSG | HSA |
| | | | | | (1-398); | | | GSSG | |
| | | | | | Y127T; | | | GSSG | |
| | | | | | L128E; | | | (SEQ | |
| | | | | | | | | ID | |
| | | | | | | | | NO: | |
| | | | | | | | | 33) | |
| 392 | | | | | hCPN1 | | | GSSG | HSA |
| | | | | | (1-398); | | | GSSG | |
| | | | | | Y127T; | | | GSSG | |
| | | | | | L128T; | | | (SEQ | |
| | | | | | | | | ID | |
| | | | | | | | | NO: | |
| | | | | | | | | 33) | |
| 393 | | | | | hCPN1 | | | GSSG | HSA |
| | | | | | (1-398); | | | GSSG | |
| | | | | | Y127R; | | | GSSG | |
| | | | | | L128T; | | | (SEQ | |
| | | | | | | | | ID | |
| | | | | | | | | NO: | |
| | | | | | | | | 33) | |
| 394 | | | | | hCPN1 | | | GSSG | HSA |
| | | | | | (1-398); | | | GSSG | |
| | | | | | Y127L; | | | GSSG | |
| | | | | | L128Y; | | | (SEQ | |
| | | | | | | | | ID | |
| | | | | | | | | NO: | |
| | | | | | | | | 33) | |
| 395 | | | | | hCPN1 | | | GSSG | HSA |
| | | | | | (1-398); | | | GSSG | |
| | | | | | Y127L; | | | GSSG | |
| | | | | | | | | (SEQ | |
| | | | | | | | | ID | |
| | | | | | | | | NO: | |
| | | | | | | | | 33) | |
| 396 | | | | | hCPN1 | | | GSSG | HSA |
| | | | | | (1-398); | | | GSSG | |
| | | | | | Y127D; | | | GSSG | |
| | | | | | L128Y; | | | (SEQ | |
| | | | | | | | | ID | |
| | | | | | | | | NO: | |
| | | | | | | | | 33) | |
| 397 | | | | | hCPN1 | | | GSSG | HSA |
| | | | | | (1-398); | | | GSSG | |
| | | | | | G243A; | | | GSSG | |
| | | | | | | | | (SEQ | |
| | | | | | | | | ID | |
| | | | | | | | | NO: | |
| | | | | | | | | 33) | |
| 398 | | | | | hCPN1 | | | GSSG | HSA |
| | | | | | (1-398); | | | GSSG | |
| | | | | | F246A; | | | GSSG | |
| | | | | | | | | (SEQ | |
| | | | | | | | | ID | |
| | | | | | | | | NO: | |
| | | | | | | | | 33) | |
| 399 | | | | | hCPN1 | | | GSSG | HSA |
| | | | | | (1-398); | | | GSSG | |
| | | | | | N250A; | | | GSSG | |
| | | | | | | | | (SEQ | |
| | | | | | | | | ID | |
| | | | | | | | | NO: | |
| | | | | | | | | 33) | |
| 400 | | | | | hCPN1 | | | GSSG | HSA |
| | | | | | (1-398); | | | GSSG | |
| | | | | | G306A; | | | GSSG | |
| | | | | | | | | (SEQ | |
| | | | | | | | | ID | |
| | | | | | | | | NO: | |
| | | | | | | | | 33) | |
| 401 | | | | | hCPN1 | | | GSSG | HSA |
| | | | | | (1-398); | | | GSSG | |
| | | | | | Y240E; | | | GSSG | |
| | | | | | | | | (SEQ | |
| | | | | | | | | ID | |
| | | | | | | | | NO: | |
| | | | | | | | | 33) | |
| 402 | | | | | hCPN1 | | | GSSG | HSA |
| | | | | | (1-398); | | | GSSG | |
| | | | | | K124E; | | | GSSG | |
| | | | | | | | | (SEQ | |
| | | | | | | | | ID | |
| | | | | | | | | NO: | |
| | | | | | | | | 33) | |
| 403 | | | | | hCPN1 | | | GSSG | HSA |
| | | | | | (1-398); | | | GSSG | |
| | | | | | K293E; | | | GSSG | |
| | | | | | | | | (SEQ | |
| | | | | | | | | ID | |
| | | | | | | | | NO: | |
| | | | | | | | | 33) | |
| 404 | | | | | hCPN1 | | | GSSG | HSA |
| | | | | | (1-398); | | | GSSG | |
| | | | | | K124E; | | | GSSG | |
| | | | | | K293E; | | | (SEQ | |
| | | | | | | | | ID | |
| | | | | | | | | NO: | |
| | | | | | | | | 33) | |
| 405 | | | | | hCPN1 | | | | |
| | | | | | (1-398); | | | | |
| | | | | | A193W; | | | | |
| | | | | | A202V; | | | | |
| | | | | | Y238F; | | | | |
| | | | | | I285L; | | | | |
| | | | | | L287I; | | | | |
| | | | | | L289Y; | | | | |
| | | | | | F314A | | | | |
| 406 | | | | | hCPN1 | | | | |
| | | | | | (1-398); | | | | |
| | | | | | D292I; | | | | |
| | | | | | K293A; | | | | |
| | | | | | F294W | | | | |
| 407 | | | | | hCPN1 | | | | |
| | | | | | (1-398); | | | | |
| | | | | | D292L; | | | | |
| | | | | | K293V; | | | | |
| | | | | | P295L | | | | |
| 408 | | | | | hCPN1 | | | | |
| | | | | | (1-398); | | | | |
| | | | | | N123Y; | | | | |
| | | | | | K124R; | | | | |
| | | | | | Y127E; | | | | |
| | | | | | D292I; | | | | |
| | | | | | K293V; | | | | |
| | | | | | F294M | | | | |
| 409 | | | | | hCPN1 | | | | |
| | | | | | (1-398); | | | | |
| | | | | | G121Q; | | | | |
| | | | | | P122W; | | | | |
| | | | | | N123D; | | | | |
| | | | | | Y127I; | | | | |
| | | | | | D292L; | | | | |
| | | | | | K293A; | | | | |
| | | | | | F294I | | | | |
| 410 | | | | | hCPN1 | | | | |
| | | | | | (1-398); | | | | |
| | | | | | G121D; | | | | |
| | | | | | P122W; | | | | |
| | | | | | N123D; | | | | |
| | | | | | Y127I; | | | | |
| | | | | | D292I; | | | | |
| | | | | | K293A; | | | | |
| | | | | | F294W | | | | |
| 411 | | | | | hCPN1 | | | | |
| | | | | | (1-398); | | | | |
| | | | | | K60F; | | | | |
| | | | | | V62S; | | | | |
| | | | | | V180Y; | | | | |
| | | | | | S192A; | | | | |
| | | | | | N276T; | | | | |
| | | | | | E284I | | | | |
| 412 | | | | | hCPN1 | | | | |
| | | | | | (1-398); | | | | |
| | | | | | K60F; | | | | |
| | | | | | V180F; | | | | |
| | | | | | S192A; | | | | |
| | | | | | N276V; | | | | |
| | | | | | E284I | | | | |
| 413 | | | | | hCPN1 | | | | |
| | | | | | (1-398); | | | | |
| | | | | | K60F; | | | | |
| | | | | | V62M; | | | | |
| | | | | | V180A; | | | | |
| | | | | | S192A; | | | | |
| | | | | | M272F; | | | | |
| | | | | | N276V; | | | | |
| | | | | | E284I | | | | |
| 414 | | | | | hCPN1 | | | | |
| | | | | | (1-320); | | | | |
| | | | | | L55N; | | | | |
| | | | | | H161R; | | | | |
| | | | | | N188D; | | | | |
| | | | | | V190K; | | | | |
| | | | | | K229E; | | | | |
| | | | | | L230W; | | | | |
| | | | | | K233H; | | | | |
| | | | | | N281D; | | | | |
| | | | | | C282S; | | | | |
| | | | | | F283Y; | | | | |
| | | | | | R308K; | | | | |
| | | | | | Q320N | | | | |
| 415 | | | | | hCPN1 | GSSG | HSA | | |
| | | | | | (1-398); | GSSG | | | |
| | | | | | S209G; | GSSG | | | |
| | | | | | ΔF210- | (SEQ | | | |
| | | | | | T220; | ID | | | |
| | | | | | A221R; | NO: | | | |
| | | | | | | 33) | | | |
| 416 | | | | | hCPN1 | GSSG | HSA | | |
| | | | | | (1-398); | GSSG | | | |
| | | | | | S209G; | GSSG | | | |
| | | | | | ΔF210- | (SEQ | | | |
| | | | | | T220; | ID | | | |
| | | | | | A221H; | NO: | | | |
| | | | | | | 33) | | | |
| 417 | | | | | hCPN1 | GSSG | HSA | | |
| | | | | | (1-398); | GSSG | | | |
| | | | | | S209G; | GSSG | | | |
| | | | | | ΔF210- | (SEQ | | | |
| | | | | | R219; | ID | | | |
| | | | | | T220P; | NO: | | | |
| | | | | | A221S; | 33) | | | |
| 418 | | | | | hCPN1 | GSSG | HSA | | |
| | | | | | (1-398); | GSSG | | | |
| | | | | | S209G; | GSSG | | | |
| | | | | | ΔF210- | (SEQ | | | |
| | | | | | R219; | ID | | | |
| | | | | | T220E; | NO: | | | |
| | | | | | A221D; | 33) | | | |
| 419 | | | | | hCPN1 | GSSG | HSA | | |
| | | | | | (1-398); | GSSG | | | |
| | | | | | S209G; | GSSG | | | |
| | | | | | ΔF210- | (SEQ | | | |
| | | | | | R219; | ID | | | |
| | | | | | T220D; | NO: | | | |
| | | | | | A221D; | 33) | | | |
| 420 | | | | | hCPN1 | GSSG | HSA | | |
| | | | | | (1-398); | GSSG | | | |
| | | | | | S209N; | GSSG | | | |
| | | | | | F210W; | (SEQ | | | |
| | | | | | E211G; | ID | | | |
| | | | | | ΔH212- | NO: | | | |
| | | | | | T220; | 33) | | | |
| | | | | | A221G; | | | | |
| 421 | | | | | hCPN1 | GSSG | HSA | | |
| | | | | | (1-398); | GSSG | | | |
| | | | | | S209A; | GSSG | | | |
| | | | | | F210E; | (SEQ | | | |
| | | | | | E211D; | ID | | | |
| | | | | | ΔH212- | NO: | | | |
| | | | | | R219; | 33) | | | |
| | | | | | T220N; | | | | |
| | | | | | A221D; | | | | |
| 422 | | | | | hCPN1 | GSSG | HSA | | |
| | | | | | (1-398); | GSSG | | | |
| | | | | | F210D; | GSSG | | | |
| | | | | | ΔE211- | (SEQ | | | |
| | | | | | R218; | ID | | | |
| | | | | | R219K; | NO: | | | |
| | | | | | T220N; | 33) | | | |
| | | | | | A221D; | | | | |
| 423 | | | | | hCPN1 | GSSG | HSA | | |
| | | | | | (1-398); | GSSG | | | |
| | | | | | F210S; | GSSG | | | |
| | | | | | ΔE211- | (SEQ | | | |
| | | | | | R219; | ID | | | |
| | | | | | A221S; | NO: | | | |
| | | | | | | 33) | | | |
| 424 | | | | | hCPN1 | GSSG | HSA | | |
| | | | | | (1-398); | GSSG | | | |
| | | | | | F210D; | GSSG | | | |
| | | | | | ΔE211- | (SEQ | | | |
| | | | | | R219; | ID | | | |
| | | | | | T220G; | NO: | | | |
| | | | | | A221D; | 33) | | | |
| 425 | | | | | hCPN1 | GSSG | HSA | | |
| | | | | | (1-398); | GSSG | | | |
| | | | | | ΔF210- | GSSG | | | |
| | | | | | R218; | (SEQ | | | |
| | | | | | R219E; | ID | | | |
| | | | | | A221G; | NO: | | | |
| | | | | | | 33) | | | |
| 426 | | | | | hCPN1 | GSSG | HSA | | |
| | | | | | (1-398); | GSSG | | | |
| | | | | | F210D; | GSSG | | | |
| | | | | | ΔE211- | (SEQ | | | |
| | | | | | R219; | ID | | | |
| | | | | | T220S; | NO: | | | |
| | | | | | A221G; | 33) | | | |
| 427 | | | | | hCPN1 | GSSG | HSA | | |
| | | | | | (1-398); | GSSG | | | |
| | | | | | F210E; | GSSG | | | |
| | | | | | E211D; | (SEQ | | | |
| | | | | | ΔH212- | ID | | | |
| | | | | | T220; | NO: | | | |
| | | | | | A221S; | 33) | | | |
| | | | | | S222E; | | | | |

TABLE 3A
Exemplary Modification Strings of hCPN1 (1-398)

| | |
|---|---|
| R37G | G121D; P122H; K124Q |
| K60F; V62S; V180Y; S192A; N276T; E284I | G121I; N123D |
| K60F; V180F; S192A; N276V; E284I | P122W; N123R; K124Y |
| K60F; V62M; V180A; S192A; M272F; N276V; E284I | P122Y; N123R; K124Y |
| K60A; V62L; M184L; S192M; M272L; N276I; E284V | K124Q |
| A193W; A202V; Y238F; I285L; L2871; L289Y; E314A | G121T; P122Y; K124Q; P125D; E297P; E298Q |
| A202V; L287I; L289T | G121T; P122Y; K124Q; P125D; E297P |
| A202I | D292I; K293I; F294W |
| A235L | K293L; E297P; E298Q |
| V1I; F3W; E74L; Q78W | D292L; K293I; P295L |
| V1I; E74L; Q78W | D292L; K293I |
| K60F; V62S; V180Y; S192A; N276T; E284I | D292N; K293A; P295T; E298S |
| K60F; V180F; S192A; N276V; E284I | D292N; K293A; E298S |
| K60F; V62M; V180A; S192A; M272F; N276V; E284I | D292I; K293A; E294W |
| K60A; V62L; M184L; S192M; M272L; N276I; E284V | D292L; K293A; E294W |
| A193W; A202V; Y238F; I285L; L2871; L289Y; E314A | D292L; K293V; E295L |
| A202V; L287I; L289T | D292L; K293V; E298D |
| A202I | K293N |
| A235L | K293L; F294W; E298L |
| V1I; F3W; E74L; Q78W | D292N; K293L; F294W |
| V1I; E74L; Q78W | N123W; K124R; P125A; Y127D; D292I; K293I; |
| A70V | F294Q |
| N68D; P125S | N123Y; K124R; P127E; D292I; K293V; F294M |
| N68D; A70V; P125S | G121T; P122Y; K124Q; P125D; Y127W; D292I; |
| E288Q | K293L; E297P |
| Y266F | N123Y; K124R; Y127D; K293I |
| Y266F; E288Q | G121D; P122S; N123T; K124I; D292N; K293A; E298S |
| A199S | G121Q; P122W; N123D; Y127I; D292L; K293A; |
| K124R; P125S | E294I |
| G121T; P122Y; K124Q; P125D | G121D; P122W; N123D; Y127I; K293A; E294W |
| N123Y; K124R | P122W; N123R; K124H; Y127S; D292L; K293V; |
| G121D; P122D; N123T; K124I | E298W |
| G121D; P122H; K124I | P122W; N123R; K124Y; D292L; K293V; E298D |
| G121R; P122E; N123Y; K124I | K124Q; K293L; F294W; E298L |
| K124D; K293L; F294W; E297D | P122R; |
| P125H | K124I; |
| P125L | G121D; N123D; K124I; |
| P122A; N123D; K124I; P125S | P125S; |
| P122A; N123D; K124S; P125S | P122G; |
| G121E; P122R; N123D; K124I; P125S | K124S; |
| G121D; P122R; N123D; K124T; P125S; G126Q | K124G; |
| N123D; K124D; P125S; G126Q | P125N; |
| N68D | P125G; |
| N68K; K293N | E297Q; |
| V201H | V180F; |
| N203D | S192A; |
| G198D | N276V; |
| G198D; V201Y | E284I; |
| A70V | K60F; |
| N68D; P125S | Y266F; E288Q |
| N68D; A70V; P125S | Y266F; E288Q |
| E288Q | Y266F; E288Q |
| Y266F | CPN1 (1-398) |
| Y266F; E288Q | Y266F; E288Q |
| A199S | A199D; |
| K124R; P125S | G121D; A199D; |
| G121T; P122Y; K124Q; P125D | N123D; A199D; |
| N123Y; K124R | G121D; N123D; A199D; |
| G121D; P122D; N123T; K124I | G121D; N123D; K124I; A199D; |
| G121D; P122H; K124I | H66K; |
| G121R; P122E; N123Y; K124I | Q78E; |
| G121D; P122H; K124Q | P125E; |
| G121I; N123D | P125D; |
| P122W; N123R; K124Y | D138K; |
| P122Y; N123R; K124Y | P296E; |
| K124Q | P296G; |
| G121D; | E298G; |
| N123D; | L300E; |
| G121D; N123D; | Q301R; |
| P122A; | Q301E; |
| R302E; | K60A; V62L; M184L; S192M; M272L; N276I; C282T; |
| D292N; | E284V; |
| F189C; | K60F; V62I; V180I; M184L; S192A; M272L; N276I; |
| R308K; E329Q; | C282T; E284I; |
| A70V; P125S; | K60G; V180F; S192M; N276M; C282T; E284V; |
| P125S; | K60G; V62I; V180I; M184Q; S192Q; N276I; C282S; |
| A70V; P125N; | E284I; |
| A70V; P125I; | K60F; S192A; N276W; E284V; |
| A70V; P125F; | K60G; S192M; N276W; E284V; |
| N68D; A70V; | K60A; S192M; N276I; E284V; |
| N68D; A70V; P125Y; | K60F; S192A; N276W; C282T; E284V; |
| N68D; P125Y; | K60G; S192A; N276W; C282T; E284V; |
| N123D; K124S; P125N; V129T; | K60F; S192A; N276W; C282L; E284V; |
| K124T; P125S; | Y266F; E288Q; |
| G121D; P122R; N123D; K124I; P125S; | Y266F; E288Q; |
| N123D; K124I; P125S; | W244A; |
| N123D; K124S; P125I; V129T; | Q247A; |
| N123D; K124A; P125S; | W249A; |
| K124A; P125S; | D253A; |
| N123D; K124A; P125N; V129T; | Y254A; |
| G121E; P122R; N123D; K124V; P125S; | D292A; |
| N123S; K124T; P125S; | E298A; |
| K60F; V62I; V180I; M184L; S192A; M272L; N276I; | E299A; |
| E284V; | R302A; |
| K60F; V62A; V180F; M184L; S192A; M272A; | K293A; |
| N276V; E284I; | P296A; |
| K60F; V62T; V180F; S192A; N276V; E284I; | G126A; |
| K60G; V62I; V180I; S192M; M272L; N276I; E284I; | Y127A; |
| K60M; V62I; V180I; M184F; S192A; M272L; N276L; | L128A; |
| E284I; | Q247L; |
| K60F; V180Y; S192A; N276T; E284L; | W244K; Q247L; |
| K60F; V62A; V180Y; M184L; S192A; M272A; | W244L; Q247M; W249Y; |
| N276T; E284I; | W244L; Q247L; W249Y; |
| K60F; V180Y; S192A; N276T; E284I; | W244F; Q247T; |
| K60Y; V180Y; M184L; S192A; M272I; N276T; E284I; | Q247M; |
| K60F; V180F; S192A; N276V; C282T; E284I; | W249Y; |
| W244D; Q247L; | K60F; V180F; S192A; S267C; S269C; N276V; E284I; |
| Y254R; | G121A; |
| Q247L; Y254R; | P122A; |
| W244K; Q247L; D253T; | N123A; |
| Q247L; D253T; | K124A; |
| D292L; | P125A; |
| R302M; | D138A; |
| R302W; | V180A; |
| R302K; | F189A; |
| R302L; | A199A; |
| R302Q; | N276A; |
| K293P; R302M; | E284A; |
| D292L; K293A; | E297A; |
| K293Y; | L300A; |
| K293Y; P296D; | Q301A; |
| K293P; R302I; | K60A; |
| K293M; P296D; | H66A; |
| Y127L; K293P; | Q78A; |
| Y127A; K293Y; | K60F; V180F; S192A; N276V; E284I; |
| D292L; K293A; | K60F; A70V; V180F; S192A; N276V; E284I |
| Y127A; K293M; | K60F; N68D; P125S; V180F; S192A; N276V; E284I |
| Y127K; K293P; | K60F; N123D; K124D; P125S; G126Q; V180F; |
| D292V; K293A; | S192A; N276V; E284I |
| K293P; | K60F; G121T; P122Y; K124Q; P125D; V180F; S192A; |
| Y127K; L128D; K293Y; | N276V; E284I |
| Y127L; L128H; K293P; | K60F; N68D; A70V; P125S; V180F; S192A; N276V; |
| Y127K; L128H; K293P; | E284I |
| L128D; | W244Y; Q247S; |
| C251S; C291S; | W244Y; Q247L; W249L; |
| T223C; A263C; | W244Y; Q247L; W249M; |
| S209C; A263C; | Q247Y; W249M; |
| S209C; S267C; | Q247Y; |
| S267C; S269C; | W244Y; Q247L; W249F; |
| K60F; V180F; S192A; T223C; A263C; N276V; E284I; | W244Y; Q247L; W249Y; |
| K60F; V180F; S192A; S209C; A263C; N276V; E284I; | Q247Y; W249L; |
| K60F; V180F; S192A; S209C; S267C; N276V; E284I; | W244Y; Q247M; W249L; |

TABLE 3B
Exemplary Modification Strings of hCPN1 (1-320)

Δ(321-398)
L55N; H161R; N188D; V190K; K229E; L230W; N281D; C282S; F283Y; R308K; Q320N
L55N; H161S; N188D; V190R; K229E; L230W; C282S; F283Y; R308K; Q320N
L55N; H161K; N188D; V190K; K229E; L230W; C282S; F283Y; R308K; Q320N
L55N; H161S; N188D; V190R; K229E; L230K; K233D; C282S; F283Y; R308K; Q320K
L55N; H161R; N188D; V190K; K229E; L230W; K233H; N281D; C282S; F283Y; R308K; Q320N
L55N; H161R; N188D; V190K; K229E; L230K; N281D; C282S; F283Y; R308K; Q320N
L55N; H161R; N188D; V190K; K229E; L230W; N281D; C282S; F283Y; R308K; Q320N
L55N; H161S; N188D; V190R; K229E; L230W; C282S; F283Y; R308K; Q320N
L55N; H161K; N188D; V190K; K229E; L230W; C282S; F283Y; R308K; Q320N
L55N; H161R; N188D; V190K; K229E; L230W; N281D; C282S; F283Y; R308K; Q320N
L55N; H161S; N188D; V190R; K229E; L230W; C282S; F283Y; R308K; Q320N
L55N; H161K; N188D; V190K; K229E; L230W; C282S; F283Y; R308K; Q320N
L55N; H161S; N188D; V190R; K229E; L230K; K233D; C282S; F283Y; R308K; Q320K
L55N; H161R; N188D; V190K; K229E; L230W; K233H; N281D; C282S; F283Y; R308K; Q320N
L55N; H161R; N188D; V190K; K229E; L230K; N281D; C282S; F283Y; R308K; Q320N
L55N; H161R; N188D; V190K; K229E; L230W; K233H; N281D; C282S; F283Y; R308K; Q320N

TABLE 3C
Exemplary Modification Strings of hCPN1 (1-438)

Y266F; E288Q
Y266F; E288Q; 398DDDDK399

TABLE 4A
Exemplary Modifications of hCPN1 (1-398)

| | | | | | | |
|---|---|---|---|---|---|---|
| V1I | V62L | Q78W | P122Y | N123T | K124H | P125A |
| F3W | V62I | Q78E | P122D | N123D | K124D | P125H |
| R37G | V62A | Q78A | P122H | N123R | K124S | P125L |
| K60F | V62T | G121T | P122E | N123W | K124T | P125N |
| K60A | H66K | G121D | P122W | N123S | K124G | P125G |
| K60G | H66A | G121R | P122S | N123A | K124A | P125E |
| K60M | N68D | G121I | P122A | K124R | K124V | P125I |
| K60Y | N68K | G121Q | P122R | K124Q | K124E | P125F |
| V62S | A70V | G121E | P122G | K124I | P125S | P125Y |
| V62M | E74L | G121A | N123Y | K124Y | P125D | G126Q |
| G126A | S192M | F246L | N276T | K293Y | Q301E | A221S |
| Y127D | S192Q | F246R | N276V | K293M | Q301A | T220E |
| P127E | A193W | Q247A | N276I | K293E | R302E | A221D |
| Y127W | G198D | Q247L | N276L | F294W | R302A | T220D |
| Y127I | A199S | Q247M | N276M | E294W | R302M | S209N |
| Y127S | A199D | Q247S | N276W | F294Q | R302W | F210W |
| Y127A | A199A | Q247Y | N276A | F294M | R302K | E211G |
| Y127L | V201H | Q247F | C282T | E294I | R302L | ΔH212- |
| Y127K | V201Y | Q247N | C282S | F294I | R302Q | T220 |
| Y127E | A202V | W249A | C282L | P295L | R302I | A221G |
| Y127T | A202I | W249L | E284I | P295T | R302D | S209A |
| Y127R | N203D | W249M | E284V | E295L | G306A | F210E |
| L128A | S209C | W249F | E284L | P296E | G306Q | E211D |
| L128D | T223C | W249Y | E284A | P296G | G306T | ΔH212- |
| L128H | A235L | W249H | I285L | P296A | G306H | R219 |
| L128T | Y238F | N250S | L287I | P296D | G306N | T220N |
| L128E | Y240E | N250A | E288Q | E297P | G306D | F210D |
| L128Y | G243E | C251S | L289Y | E297D | G306F | ΔE211- |
| V129T | G243A | D253A | L289T | E297Q | G306W | R218 |
| D138K | G243Y | D253T | C291S | E297A | R308K | R219K |
| D138A | G243W | D253L | D292I | E298Q | E314A | F210S |
| V180Y | W244A | D253N | D292L | E298S | F314A | ΔE211- |
| V180F | W244K | Y254A | D292N | E298D | E329Q | R219 |
| V180A | W244L | A263C | D292A | E298L | S209G | T220G |
| V180I | W244Y | Y266F | D292V | E298W | ΔF210- | ΔF210- |
| M184L | W244D | S267C | K293I | E298G | T220 | R218 |
| M184F | W244M | S269C | K293L | E298A | A221R | R219E |
| M184Q | W244I | M272F | K293A | E299A | A221H | T220S |
| F189C | F246Y | M272L | K293V | L300E | ΔF210- | S222E |
| F189A | F246W | M272A | K293N | L300A | R219 | |
| S192A | F246A | M272I | K293P | Q301R | T220P | |

TABLE 4B
Exemplary Modifications of hCPN1 (1-320)

| | | | | | | |
|---|---|---|---|---|---|---|
| L55N | H161S | V190R | L230W | N281D | R308K | Δ(321-398) |
| H161K | N188D | K229E | K233D | C282S | Q320K | |
| H161R | V190K | L230K | K233H | F283Y | Q320N | |

TABLE 4C
Exemplary Modifications of hCPN1 (1-438)

Y266F
E288Q
398DDDDK399 (SEQ ID NO: 44)

TABLE 5
Amino Acid Sequences of Exemplary
Fusion Constructs

| | | Signal | |
|---|---|---|---|
| | | Sequence | Full |
| | | SEQ | Pro- |
| Const. | Signal Sequence | ID | tein |
| No. | (Optional) | (Optional) | SEQ ID |
| 1 | MKWVTFISLLFLFSSAYS | 487 | 73 |
| 2 | MTRLTVLALLAGLLASSRA | 19 | 74 |
| 3 | MYRMQLLSCIALSLALVTNS | 20 | 75 |
| 4 | MGWSLILLFLVAVATRVHS | 21 | 76 |
| 5 | MGWSLILLFLVAVATRVHS | 21 | 77 |
| 6 | MGWSLILLFLVAVATRVHS | 21 | 78 |
| 7 | MGWSLILLFLVAVATRVHS | 21 | 79 |
| 8 | MGWSLILLFLVAVATRVHS | 21 | 80 |
| 9 | MGWSLILLFLVAVATRVHS | 21 | 81 |
| 10 | MGWSLILLFLVAVATRVHS | 21 | 82 |
| 11 | MGWSLILLFLVAVATRVHS | 21 | 83 |
| 12 | MGWSLILLFLVAVATRVHS | 21 | 84 |
| 13 | MGWSLILLFLVAVATRVHS | 21 | 85 |
| 14 | MGWSLILLFLVAVATRVHS | 21 | 86 |
| 15 | MGWSLILLFLVAVATRVHS | 21 | 87 |
| 16 | MGWSLILLFLVAVATRVHS | 21 | 88 |
| 17 | MGWSLILLFLVAVATRVHS | 21 | 89 |
| 18 | MGWSLILLFLVAVATRVHS | 21 | 90 |
| 19 | MGWSLILLFLVAVATRVHS | 21 | 91 |
| 20 | MGWSLILLFLVAVATRVHS | 21 | 92 |
| 21 | MGWSLILLFLVAVATRVHS | 21 | 93 |
| 22 | MGWSLILLFLVAVATRVHS | 21 | 94 |
| 23 | MGWSLILLFLVAVATRVHS | 21 | 95 |
| 24 | MGWSLILLFLVAVATRVHS | 21 | 96 |
| 25 | MGWSLILLFLVAVATRVHS | 21 | 97 |
| 26 | MGWSLILLFLVAVATRVHS | 21 | 98 |
| 27 | MGWSLILLFLVAVATRVHS | 21 | 99 |
| 28 | MGWSLILLFLVAVATRVHS | 21 | 100 |
| 29 | MGWSLILLFLVAVATRVHS | 21 | 101 |
| 30 | MGWSLILLFLVAVATRVHS | 21 | 102 |
| 31 | MGWSLILLFLVAVATRVHS | 21 | 103 |
| 32 | MGWSLILLFLVAVATRVHS | 21 | 104 |
| 33 | MGWSLILLFLVAVATRVHS | 21 | 105 |
| 34 | MGWSLILLFLVAVATRVHS | 21 | 106 |
| 35 | MGWSLILLFLVAVATRVHS | 21 | 107 |
| 36 | MGWSLILLFLVAVATRVHS | 21 | 108 |
| 37 | no signal seq | | 109 |
| 38 | MGWSLILLFLVAVATRVHS | 21 | 110 |
| 39 | MGWSLILLFLVAVATRVHS | 21 | 111 |
| 40 | MGWSLILLFLVAVATRVHS | 21 | 112 |
| 41 | TMGWSLILLFLVAVATRVHS | 488 | 113 |
| 42 | TMGWSLILLFLVAVATRVHS | 488 | 114 |
| 43 | TMGWSLILLFLVAVATRVHS | 488 | 115 |
| 44 | TMGWSLILLFLVAVATRVHS | 488 | 116 |
| 45 | TMGWSLILLFLVAVATRVHS | 488 | 117 |
| 46 | TMGWSLILLFLVAVATRVHS | 488 | 118 |
| 47 | TMGWSLILLFLVAVATRVHS | 488 | 119 |
| 48 | TMGWSLILLFLVAVATRVHS | 488 | 120 |
| 49 | TMGWSLILLFLVAVATRVHS | 488 | 121 |
| 50 | TMGWSLILLFLVAVATRVHS | 488 | 122 |
| 51 | TMGWSLILLFLVAVATRVHS | 488 | 123 |
| 52 | TMGWSLILLFLVAVATRVHS | 488 | 124 |
| 53 | TMGWSLILLFLVAVATRVHS | 488 | 125 |
| 54 | TMGWSLILLFLVAVATRVHS | 488 | 126 |
| 55 | TMGWSLILLFLVAVATRVHS | 488 | 127 |
| 56 | TMGWSLILLFLVAVATRVHS | 488 | 128 |
| 57 | TMGWSLILLFLVAVATRVHS | 488 | 129 |
| 58 | TMGWSLILLFLVAVATRVHS | 488 | 130 |
| 59 | TMGWSLILLFLVAVATRVHS | 488 | 131 |
| 60 | TMGWSLILLFLVAVATRVHS | 488 | 132 |
| 61 | TMGWSLILLFLVAVATRVHS | 488 | 133 |
| 62 | TMGWSLILLFLVAVATRVHS | 488 | 134 |
| 63 | TMGWSLILLFLVAVATRVHS | 488 | 135 |
| 64 | TMGWSLILLFLVAVATRVHS | 488 | 136 |
| 65 | TMGWSLILLFLVAVATRVHS | 488 | 137 |
| 66 | TMGWSLILLFLVAVATRVHS | 488 | 138 |
| 67 | TMGWSLILLFLVAVATRVHS | 488 | 139 |
| 68 | TMGWSLILLFLVAVATRVHS | 488 | 140 |
| 69 | TMGWSLILLFLVAVATRVHS | 488 | 141 |
| 70 | TMGWSLILLFLVAVATRVHS | 488 | 142 |
| 71 | TMGWSLILLFLVAVATRVHS | 488 | 143 |
| 72 | TMGWSLILLFLVAVATRVHS | 488 | 144 |
| 73 | TMGWSLILLFLVAVATRVHS | 488 | 145 |
| 74 | TMGWSLILLFLVAVATRVHS | 488 | 146 |
| 75 | TMGWSLILLFLVAVATRVHS | 488 | 147 |
| 76 | TMGWSLILLFLVAVATRVHS | 488 | 148 |
| 77 | TMGWSLILLFLVAVATRVHS | 488 | 149 |
| 78 | TMGWSLILLFLVAVATRVHS | 488 | 150 |
| 79 | TMGWSLILLFLVAVATRVHS | 488 | 151 |
| 80 | TMGWSLILLFLVAVATRVHS | 488 | 152 |
| 81 | TMGWSLILLFLVAVATRVHS | 488 | 153 |
| 82 | TMGWSLILLFLVAVATRVHS | 488 | 154 |
| 83 | TMGWSLILLFLVAVATRVHS | 488 | 155 |
| 84 | TMGWSLILLFLVAVATRVHS | 488 | 156 |
| 85 | TMGWSLILLFLVAVATRVHS | 488 | 157 |
| 86 | TMGWSLILLFLVAVATRVHS | 488 | 158 |
| 87 | TTTMGWSLILLFLVAVATRVHS | 489 | 159 |
| 88 | TTTMGWSLILLFLVAVATRVHS | 489 | 160 |
| 89 | TTTMGWSLILLFLVAVATRVHS | 489 | 161 |
| 90 | TTTMGWSLILLFLVAVATRVHS | 489 | 162 |
| 91 | TTTMGWSLILLFLVAVATRVHS | 489 | 163 |
| 92 | TTTMGWSLILLFLVAVATRVHS | 489 | 164 |
| 93 | TTTMGWSLILLFLVAVATRVHS | 489 | 165 |
| 94 | TTTMGWSLILLFLVAVATRVHS | 489 | 166 |
| 95 | TTTMGWSLILLFLVAVATRVHS | 489 | 167 |
| 96 | TTTMGWSLILLFLVAVATRVHS | 489 | 168 |
| 97 | TTTMGWSLILLFLVAVATRVHS | 489 | 169 |
| 98 | TTTMGWSLILLFLVAVATRVHS | 489 | 170 |
| 99 | TTTMGWSLILLFLVAVATRVHS | 489 | 171 |
| 100 | TTTMGWSLILLFLVAVATRVHS | 489 | 172 |
| 101 | TTTMGWSLILLFLVAVATRVHS | 489 | 173 |
| 102 | TTTMGWSLILLFLVAVATRVHS | 489 | 174 |
| 103 | TTTMGWSLILLFLVAVATRVHS | 489 | 175 |
| 104 | TTTMGWSLILLFLVAVATRVHS | 489 | 176 |
| 105 | TTTMGWSLILLFLVAVATRVHS | 489 | 177 |
| 106 | TTTMGWSLILLFLVAVATRVHS | 489 | 178 |
| 107 | TTTMGWSLILLFLVAVATRVHS | 489 | 179 |
| 108 | TTTMGWSLILLFLVAVATRVHS | 489 | 180 |
| 109 | TTTMGWSLILLFLVAVATRVHS | 489 | 181 |
| 110 | TTTMGWSLILLFLVAVATRVHS | 489 | 182 |
| 111 | TTTMGWSLILLFLVAVATRVHS | 489 | 183 |
| 112 | TTTMGWSLILLFLVAVATRVHS | 489 | 184 |
| 113 | TTTMGWSLILLFLVAVATRVHS | 489 | 185 |
| 114 | TTTMGWSLILLFLVAVATRVHS | 489 | 186 |
| 115 | TTTMGWSLILLFLVAVATRVHS | 489 | 187 |
| 116 | TTTMGWSLILLFLVAVATRVHS | 489 | 188 |
| 117 | TTTMGWSLILLFLVAVATRVHS | 489 | 189 |
| 118 | TTTMGWSLILLFLVAVATRVHS | 489 | 190 |
| 119 | TTTMGWSLILLFLVAVATRVHS | 489 | 191 |
| 120 | TTTMGWSLILLFLVAVATRVHS | 489 | 192 |
| 121 | TTTMGWSLILLFLVAVATRVHS | 489 | 193 |
| 122 | TTTMGWSLILLFLVAVATRVHS | 489 | 194 |
| 123 | TTTMGWSLILLFLVAVATRVHS | 489 | 195 |
| 124 | TTTMGWSLILLFLVAVATRVHS | 489 | 196 |
| 125 | TTTMGWSLILLFLVAVATRVHS | 489 | 197 |
| 126 | TTTMGWSLILLFLVAVATRVHS | 489 | 198 |
| 127 | TTTMGWSLILLFLVAVATRVHS | 489 | 199 |
| 128 | TTTMGWSLILLFLVAVATRVHS | 489 | 200 |
| 129 | TTTMGWSLILLFLVAVATRVHS | 489 | 201 |
| 130 | TTTMGWSLILLFLVAVATRVHS | 489 | 202 |
| 131 | TTTMGWSLILLFLVAVATRVHS | 489 | 203 |
| 132 | TTTMGWSLILLFLVAVATRVHS | 489 | 204 |
| 133 | TTTMGWSLILLFLVAVATRVHS | 489 | 205 |
| 134 | TTTMGWSLILLFLVAVATRVHS | 489 | 206 |
| 135 | TTTMGWSLILLFLVAVATRVHS | 489 | 207 |
| 136 | TTTMGWSLILLFLVAVATRVHS | 489 | 208 |
| 137 | TTTMGWSLILLFLVAVATRVHS | 489 | 209 |
| 138 | TMGWSLILLFLVAVATRVHS | 488 | 210 |
| 139 | TMGWSLILLFLVAVATRVHS | 488 | 211 |
| 140 | TMGWSLILLFLVAVATRVHS | 488 | 212 |
| 141 | TMGWSLILLFLVAVATRVHS | 488 | 213 |
| 142 | TMGWSLILLFLVAVATRVHS | 488 | 214 |
| 143 | TMGWSLILLFLVAVATRVHS | 488 | 215 |
| 144 | TTTMGWSLILLFLVAVATRVHS | 489 | 216 |
| 145 | TTTMGWSLILLFLVAVATRVHS | 489 | 217 |
| 146 | TTTMGWSLILLFLVAVATRVHS | 489 | 218 |
| 147 | TTTMGWSLILLFLVAVATRVHS | 489 | 219 |
| 148 | TTTMGWSLILLFLVAVATRVHS | 489 | 220 |
| 149 | TTTMGWSLILLFLVAVATRVHS | 489 | 221 |
| 150 | TTTMGWSLILLFLVAVATRVHS | 489 | 222 |
| 151 | TTTMGWSLILLFLVAVATRVHS | 489 | 223 |
| 152 | TTTMGWSLILLFLVAVATRVHS | 489 | 224 |
| 153 | TTTMGWSLILLFLVAVATRVHS | 489 | 225 |
| 154 | TTTMGWSLILLFLVAVATRVHS | 489 | 226 |
| 155 | TTTMGWSLILLFLVAVATRVHS | 489 | 227 |
| 156 | TTTMGWSLILLFLVAVATRVHS | 489 | 228 |
| 157 | TTTMGWSLILLFLVAVATRVHS | 489 | 229 |
| 158 | TTTMGWSLILLFLVAVATRVHS | 489 | 230 |
| 159 | AATMGWSLILLFLVAVATRVHS | 486 | 231 |
| 160 | AATMGWSLILLFLVAVATRVHS | 486 | 232 |
| 161 | AATMGWSLILLFLVAVATRVHS | 486 | 233 |
| 162 | AATMGWSLILLFLVAVATRVHS | 486 | 234 |
| 163 | AATMGWSLILLFLVAVATRVHS | 486 | 235 |
| 164 | AATMGWSLILLFLVAVATRVHS | 486 | 236 |
| 165 | AATMGWSLILLFLVAVATRVHS | 486 | 237 |
| 166 | AATMGWSLILLFLVAVATRVHS | 486 | 238 |
| 167 | AATMGWSLILLFLVAVATRVHS | 486 | 239 |
| 168 | AATMGWSLILLFLVAVATRVHS | 486 | 240 |
| 169 | AATMGWSLILLFLVAVATRVHS | 486 | 241 |
| 170 | AATMGWSLILLFLVAVATRVHS | 486 | 242 |
| 171 | AATMGWSLILLFLVAVATRVHS | 486 | 243 |
| 172 | AATMGWSLILLFLVAVATRVHS | 486 | 244 |
| 173 | AATMGWSLILLFLVAVATRVHS | 486 | 245 |
| 174 | AATMGWSLILLFLVAVATRVHS | 486 | 246 |
| 175 | AATMGWSLILLFLVAVATRVHS | 486 | 247 |
| 176 | AATMGWSLILLFLVAVATRVHS | 486 | 248 |
| 177 | AATMGWSLILLFLVAVATRVHS | 486 | 249 |
| 178 | AATMGWSLILLFLVAVATRVHS | 486 | 250 |
| 179 | AATMGWSLILLFLVAVATRVHS | 486 | 251 |
| 180 | AATMGWSLILLFLVAVATRVHS | 486 | 252 |
| 181 | AATMGWSLILLFLVAVATRVHS | 486 | 253 |
| 182 | AATMGWSLILLFLVAVATRVHS | 486 | 254 |
| 183 | AATMGWSLILLFLVAVATRVHS | 486 | 255 |
| 184 | AATMGWSLILLFLVAVATRVHS | 486 | 256 |
| 185 | AATMGWSLILLFLVAVATRVHS | 486 | 257 |
| 186 | AATMGWSLILLFLVAVATRVHS | 486 | 258 |
| 187 | AATMGWSLILLFLVAVATRVHS | 486 | 259 |
| 188 | AATMGWSLILLFLVAVATRVHS | 486 | 260 |
| 189 | AATMGWSLILLFLVAVATRVHS | 486 | 261 |
| 190 | AATMGWSLILLFLVAVATRVHS | 486 | 262 |
| 191 | AATMGWSLILLFLVAVATRVHS | 486 | 263 |
| 192 | AATMGWSLILLFLVAVATRVHS | 486 | 264 |
| 193 | AATMGWSLILLFLVAVATRVHS | 486 | 265 |
| 194 | AATMGWSLILLFLVAVATRVHS | 486 | 266 |
| 195 | AATMGWSLILLFLVAVATRVHS | 486 | 267 |
| 196 | AATMGWSLILLFLVAVATRVHS | 486 | 268 |
| 197 | AATMGWSLILLFLVAVATRVHS | 486 | 269 |
| 198 | AATMGWSLILLFLVAVATRVHS | 486 | 270 |
| 199 | AATMGWSLILLFLVAVATRVHS | 486 | 271 |
| 200 | AATMGWSLILLFLVAVATRVHS | 486 | 272 |
| 201 | AATMGWSLILLFLVAVATRVHS | 486 | 273 |
| 202 | AATMGWSLILLFLVAVATRVHS | 486 | 274 |
| 203 | AATMGWSLILLFLVAVATRVHS | 486 | 275 |
| 204 | AATMGWSLILLFLVAVATRVHS | 486 | 276 |
| 205 | AATMGWSLILLFLVAVATRVHS | 486 | 277 |
| 206 | AATMGWSLILLFLVAVATRVHS | 486 | 278 |
| 207 | AATMGWSLILLFLVAVATRVHS | 486 | 279 |
| 208 | AATMGWSLILLFLVAVATRVHS | 486 | 280 |
| 209 | AATMGWSLILLFLVAVATRVHS | 486 | 281 |
| 210 | AATMGWSLILLFLVAVATRVHS | 486 | 282 |
| 211 | AATMGWSLILLFLVAVATRVHS | 486 | 283 |
| 212 | AATMGWSLILLFLVAVATRVHS | 486 | 284 |
| 213 | AATMGWSLILLFLVAVATRVHS | 486 | 285 |
| 214 | AATMGWSLILLFLVAVATRVHS | 486 | 286 |
| 215 | GSATMETDTLLLWVLLLWVPGSTG | 490 | 287 |
| 216 | AATMGWSLILLFLVAVATRVHS | 486 | 288 |
| 217 | AATMGWSLILLFLVAVATRVHS | 486 | 289 |
| 218 | AATMGWSLILLFLVAVATRVHS | 486 | 290 |
| 219 | AATMGWSLILLFLVAVATRVHS | 486 | 291 |
| 220 | AATMGWSLILLFLVAVATRVHS | 486 | 292 |
| 22 | AATMGWSLILLFLVAVATRVHS | 486 | 293 |
| 222 | AATMGWSLILLFLVAVATRVHS | 486 | 294 |
| 223 | AATMGWSLILLFLVAVATRVHS | 486 | 295 |
| 224 | AATMGWSLILLFLVAVATRVHS | 486 | 296 |
| 225 | AATMGWSLILLFLVAVATRVHS | 486 | 297 |
| 226 | AATMGWSLILLFLVAVATRVHS | 486 | 298 |
| 227 | AATMGWSLILLFLVAVATRVHS | 486 | 299 |
| 228 | AATMGWSLILLFLVAVATRVHS | 486 | 300 |
| 229 | AATMGWSLILLFLVAVATRVHS | 486 | 301 |
| 230 | AATMGWSLILLFLVAVATRVHS | 486 | 302 |
| 231 | AATMGWSLILLFLVAVATRVHS | 486 | 303 |
| 232 | AATMGWSLILLFLVAVATRVHS | 486 | 304 |
| 233 | AATMGWSLILLFLVAVATRVHS | 486 | 305 |
| 234 | AATMGWSLILLFLVAVATRVHS | 486 | 306 |
| 235 | AATMGWSLILLFLVAVATRVHS | 486 | 307 |
| 236 | AATMGWSLILLFLVAVATRVHS | 486 | 308 |
| 237 | AATMGWSLILLFLVAVATRVHS | 486 | 309 |
| 238 | AATMGWSLILLFLVAVATRVHS | 486 | 310 |
| 239 | AATMGWSLILLFLVAVATRVHS | 486 | 311 |
| 240 | AATMGWSLILLFLVAVATRVHS | 486 | 312 |
| 241 | AATMGWSLILLFLVAVATRVHS | 486 | 313 |
| 242 | AATMGWSLILLFLVAVATRVHS | 486 | 314 |
| 243 | AATMGWSLILLFLVAVATRVHS | 486 | 315 |
| 244 | AATMGWSLILLFLVAVATRVHS | 486 | 316 |
| 245 | AATMGWSLILLFLVAVATRVHS | 486 | 317 |
| 246 | AATMGWSLILLFLVAVATRVHS | 486 | 318 |
| 247 | AATMGWSLILLFLVAVATRVHS | 486 | 319 |
| 248 | AATMGWSLILLFLVAVATRVHS | 486 | 320 |
| 249 | AATMGWSLILLFLVAVATRVHS | 486 | 321 |
| 250 | AATMGWSLILLFLVAVATRVHS | 486 | 322 |
| 251 | AATMGWSLILLFLVAVATRVHS | 486 | 323 |
| 252 | AATMGWSLILLFLVAVATRVHS | 486 | 324 |
| 253 | AATMGWSLILLFLVAVATRVHS | 486 | 325 |
| 254 | AATMGWSLILLFLVAVATRVHS | 486 | 326 |
| 255 | AATMGWSLILLFLVAVATRVHS | 486 | 327 |
| 256 | AATMGWSLILLFLVAVATRVHS | 486 | 328 |
| 257 | AATMGWSLILLFLVAVATRVHS | 486 | 329 |
| 258 | AATMGWSLILLFLVAVATRVHS | 486 | 330 |
| 259 | AATMGWSLILLFLVAVATRVHS | 486 | 331 |
| 260 | AATMGWSLILLFLVAVATRVHS | 486 | 332 |
| 261 | AATMGWSLILLFLVAVATRVHS | 486 | 333 |
| 262 | AATMGWSLILLFLVAVATRVHS | 486 | 334 |
| 263 | AATMGWSLILLFLVAVATRVHS | 486 | 335 |
| 264 | AATMGWSLILLFLVAVATRVHS | 486 | 336 |
| 265 | AATMGWSLILLFLVAVATRVHS | 486 | 337 |
| 266 | AATMGWSLILLFLVAVATRVHS | 486 | 338 |
| 267 | AATMGWSLILLFLVAVATRVHS | 486 | 339 |
| 268 | AATMGWSLILLFLVAVATRVHS | 486 | 340 |
| 269 | AATMGWSLILLFLVAVATRVHS | 486 | 341 |
| 270 | AATMGWSLILLFLVAVATRVHS | 486 | 342 |
| 271 | AATMGWSLILLFLVAVATRVHS | 486 | 343 |
| 272 | AATMGWSLILLFLVAVATRVHS | 486 | 344 |
| 273 | AATMGWSLILLFLVAVATRVHS | 486 | 345 |
| 274 | AATMGWSLILLFLVAVATRVHS | 486 | 346 |
| 275 | AATMGWSLILLFLVAVATRVHS | 486 | 347 |
| 276 | AATMGWSLILLFLVAVATRVHS | 486 | 348 |
| 277 | AATMGWSLILLFLVAVATRVHS | 486 | 349 |
| 278 | AATMGWSLILLFLVAVATRVHS | 486 | 350 |
| 279 | AATMGWSLILLFLVAVATRVHS | 486 | 351 |
| 280 | AATMGWSLILLFLVAVATRVHS | 486 | 352 |
| 281 | AATMGWSLILLFLVAVATRVHS | 486 | 353 |
| 282 | AATMGWSLILLFLVAVATRVHS | 486 | 354 |
| 283 | AATMGWSLILLFLVAVATRVHS | 486 | 355 |
| 284 | AATMGWSLILLFLVAVATRVHS | 486 | 356 |
| 285 | AATMGWSLILLFLVAVATRVHS | 486 | 357 |
| 286 | AATMGWSLILLFLVAVATRVHS | 486 | 358 |
| 287 | AATMGWSLILLFLVAVATRVHS | 486 | 359 |
| 288 | AATMGWSLILLFLVAVATRVHS | 486 | 360 |
| 289 | AATMGWSLILLFLVAVATRVHS | 486 | 361 |
| 290 | AATMGWSLILLFLVAVATRVHS | 486 | 362 |
| 291 | AATMGWSLILLFLVAVATRVHS | 486 | 363 |
| 292 | AATMGWSLILLFLVAVATRVHS | 486 | 364 |
| 293 | AATMGWSLILLFLVAVATRVHS | 486 | 365 |
| 294 | AATMGWSLILLFLVAVATRVHS | 486 | 366 |
| 295 | AATMGWSLILLFLVAVATRVHS | 486 | 367 |
| 296 | AATMGWSLILLFLVAVATRVHS | 486 | 368 |
| 297 | AATMGWSLILLFLVAVATRVHS | 486 | 369 |
| 298 | AATMGWSLILLFLVAVATRVHS | 486 | 370 |
| 299 | AATMGWSLILLFLVAVATRVHS | 486 | 371 |
| 300 | AATMGWSLILLFLVAVATRVHS | 486 | 372 |
| 301 | AATMGWSLILLFLVAVATRVHS | 486 | 373 |
| 302 | AATMGWSLILLFLVAVATRVHS | 486 | 374 |
| 303 | AATMGWSLILLFLVAVATRVHS | 486 | 375 |
| 304 | AATMGWSLILLFLVAVATRVHS | 486 | 376 |
| 305 | AATMGWSLILLFLVAVATRVHS | 486 | 377 |
| 306 | AATMGWSLILLFLVAVATRVHS | 486 | 378 |
| 307 | AATMGWSLILLFLVAVATRVHS | 486 | 379 |
| 308 | AATMGWSLILLFLVAVATRVHS | 486 | 380 |
| 309 | AATMGWSLILLFLVAVATRVHS | 486 | 381 |
| 310 | AATMGWSLILLFLVAVATRVHS | 486 | 382 |
| 311 | AATMGWSLILLFLVAVATRVHS | 486 | 383 |
| 312 | AATMGWSLILLFLVAVATRVHS | 486 | 384 |
| 313 | AATMGWSLILLFLVAVATRVHS | 486 | 385 |
| 314 | AATMGWSLILLFLVAVATRVHS | 486 | 386 |
| 315 | AATMGWSLILLFLVAVATRVHS | 486 | 387 |
| 316 | AATMGWSLILLFLVAVATRVHS | 486 | 388 |
| 317 | AATMGWSLILLFLVAVATRVHS | 486 | 389 |
| 318 | AATMGWSLILLFLVAVATRVHS | 486 | 390 |
| 319 | AATMGWSLILLFLVAVATRVHS | 486 | 391 |
| 320 | AATMGWSLILLFLVAVATRVHS | 486 | 392 |
| 321 | AATMGWSLILLFLVAVATRVHS | 486 | 393 |
| 322 | AATMGWSLILLFLVAVATRVHS | 486 | 394 |
| 323 | AATMGWSLILLFLVAVATRVHS | 486 | 395 |
| 324 | AATMGWSLILLFLVAVATRVHS | 486 | 396 |
| 325 | AATMGWSLILLFLVAVATRVHS | 486 | 397 |
| 326 | AATMGWSLILLFLVAVATRVHS | 486 | 398 |
| 327 | AATMGWSLILLFLVAVATRVHS | 486 | 399 |
| 328 | AATMGWSLILLFLVAVATRVHS | 486 | 400 |
| 329 | AATMGWSLILLFLVAVATRVHS | 486 | 401 |
| 330 | AATMGWSLILLFLVAVATRVHS | 486 | 402 |
| 331 | AATMGWSLILLFLVAVATRVHS | 486 | 403 |
| 332 | AATMGWSLILLFLVAVATRVHS | 486 | 404 |
| 333 | AATMGWSLILLFLVAVATRVHS | 486 | 405 |
| 334 | AATMGWSLILLFLVAVATRVHS | 486 | 406 |
| 335 | AATMGWSLILLFLVAVATRVHS | 486 | 407 |
| 336 | AATMGWSLILLFLVAVATRVHS | 486 | 408 |
| 337 | AATMGWSLILLFLVAVATRVHS | 486 | 409 |
| 338 | AATMGWSLILLFLVAVATRVHS | 486 | 410 |
| 339 | AATMGWSLILLFLVAVATRVHS | 486 | 411 |
| 340 | AATMGWSLILLFLVAVATRVHS | 486 | 412 |
| 341 | AATMGWSLILLFLVAVATRVHS | 486 | 413 |
| 342 | AATMGWSLILLFLVAVATRVHS | 486 | 414 |
| 343 | AATMGWSLILLFLVAVATRVHS | 486 | 415 |
| 344 | AATMGWSLILLFLVAVATRVHS | 486 | 416 |
| 345 | AATMGWSLILLFLVAVATRVHS | 486 | 417 |
| 346 | AATMGWSLILLFLVAVATRVHS | 486 | 418 |
| 347 | AATMGWSLILLFLVAVATRVHS | 486 | 419 |
| 348 | AATMGWSLILLFLVAVATRVHS | 486 | 420 |
| 349 | AATMGWSLILLFLVAVATRVHS | 486 | 421 |
| 350 | AATMGWSLILLFLVAVATRVHS | 486 | 422 |
| 351 | AATMGWSLILLFLVAVATRVHS | 486 | 423 |
| 352 | AATMGWSLILLFLVAVATRVHS | 486 | 424 |
| 353 | AATMGWSLILLFLVAVATRVHS | 486 | 425 |
| 354 | AATMGWSLILLFLVAVATRVHS | 486 | 426 |
| 355 | AATMGWSLILLFLVAVATRVHS | 486 | 427 |
| 356 | AATMGWSLILLFLVAVATRVHS | 486 | 428 |
| 357 | AATMGWSLILLFLVAVATRVHS | 486 | 429 |
| 358 | AATMGWSLILLFLVAVATRVHS | 486 | 430 |
| 359 | AATMGWSLILLFLVAVATRVHS | 486 | 431 |
| 360 | AATMGWSLILLFLVAVATRVHS | 486 | 432 |
| 361 | AATMGWSLILLFLVAVATRVHS | 486 | 433 |
| 362 | AATMGWSLILLFLVAVATRVHS | 486 | 434 |
| 363 | AATMGWSLILLFLVAVATRVHS | 486 | 435 |
| 364 | AATMGWSLILLFLVAVATRVHS | 486 | 436 |
| 365 | AATMGWSLILLFLVAVATRVHS | 486 | 437 |
| 366 | AATMGWSLILLFLVAVATRVHS | 486 | 438 |
| 367 | AATMGWSLILLFLVAVATRVHS | 486 | 439 |
| 368 | AATMGWSLILLFLVAVATRVHS | 486 | 440 |
| 369 | AATMGWSLILLFLVAVATRVHS | 486 | 441 |
| 370 | AATMGWSLILLFLVAVATRVHS | 486 | 442 |
| 371 | AATMGWSLILLFLVAVATRVHS | 486 | 443 |
| 372 | AATMGWSLILLFLVAVATRVHS | 486 | 444 |
| 373 | AATMGWSLILLFLVAVATRVHS | 486 | 445 |
| 374 | AATMGWSLILLFLVAVATRVHS | 486 | 446 |
| 375 | AATMGWSLILLFLVAVATRVHS | 486 | 447 |
| 376 | AATMGWSLILLFLVAVATRVHS | 486 | 448 |
| 377 | AATMGWSLILLFLVAVATRVHS | 486 | 449 |
| 378 | AATMGWSLILLFLVAVATRVHS | 486 | 450 |
| 379 | AATMGWSLILLFLVAVATRVHS | 486 | 451 |
| 380 | AATMGWSLILLFLVAVATRVHS | 486 | 452 |
| 381 | AATMGWSLILLFLVAVATRVHS | 486 | 453 |
| 382 | AATMGWSLILLFLVAVATRVHS | 486 | 454 |
| 383 | AATMGWSLILLFLVAVATRVHS | 486 | 455 |
| 384 | AATMGWSLILLFLVAVATRVHS | 486 | 456 |
| 385 | AATMGWSLILLFLVAVATRVHS | 486 | 457 |
| 386 | AATMGWSLILLFLVAVATRVHS | 486 | 458 |
| 387 | AATMGWSLILLFLVAVATRVHS | 486 | 459 |
| 388 | AATMGWSLILLFLVAVATRVHS | 486 | 460 |
| 389 | AATMGWSLILLFLVAVATRVHS | 486 | 461 |
| 390 | AATMGWSLILLFLVAVATRVHS | 486 | 462 |
| 391 | AATMGWSLILLFLVAVATRVHS | 486 | 463 |
| 392 | AATMGWSLILLFLVAVATRVHS | 486 | 464 |
| 393 | AATMGWSLILLFLVAVATRVHS | 486 | 465 |
| 394 | AATMGWSLILLFLVAVATRVHS | 486 | 466 |
| 395 | AATMGWSLILLFLVAVATRVHS | 486 | 467 |
| 396 | AATMGWSLILLFLVAVATRVHS | 486 | 468 |
| 397 | AATMGWSLILLFLVAVATRVHS | 486 | 469 |
| 398 | AATMGWSLILLFLVAVATRVHS | 486 | 470 |
| 399 | AATMGWSLILLFLVAVATRVHS | 486 | 471 |
| 400 | AATMGWSLILLFLVAVATRVHS | 486 | 472 |
| 415 | AATMGWSLILLFLVAVATRVHS | 486 | 473 |
| 416 | AATMGWSLILLFLVAVATRVHS | 486 | 474 |
| 417 | AATMGWSLILLFLVAVATRVHS | 486 | 475 |
| 418 | AATMGWSLILLFLVAVATRVHS | 486 | 476 |
| 419 | AATMGWSLILLFLVAVATRVHS | 486 | 477 |
| 420 | AATMGWSLILLFLVAVATRVHS | 486 | 478 |
| 421 | AATMGWSLILLFLVAVATRVHS | 486 | 479 |
| 422 | AATMGWSLILLFLVAVATRVHS | 486 | 480 |
| 423 | AATMGWSLILLFLVAVATRVHS | 486 | 481 |
| 424 | AATMGWSLILLFLVAVATRVHS | 486 | 482 |
| 425 | AATMGWSLILLFLVAVATRVHS | 486 | 483 |
| 426 | AATMGWSLILLFLVAVATRVHS | 486 | 484 |
| 427 | AATMGWSLILLFLVAVATRVHS | 486 | 485 |

The present description sets forth numerous exemplary configurations, methods, parameters, and the like. It should be recognized, however, that such description is not intended as a limitation on the scope of the present disclosure, but is instead provided as a description of exemplary embodiments. Embodiments of the present subject matter described above may be beneficial alone or in combination, with one or more other aspects or embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

The following Enumerated Embodiments and Examples are merely illustrative and are not meant to limit any aspects of the present disclosure in any way.

### ENUMERATED EMBODIMENTS

### Set I

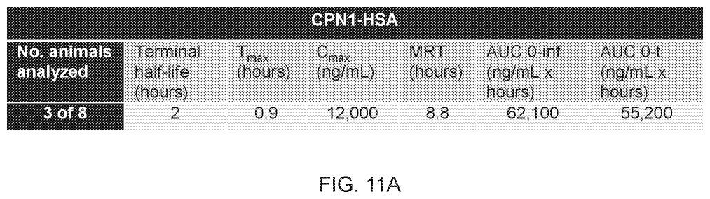

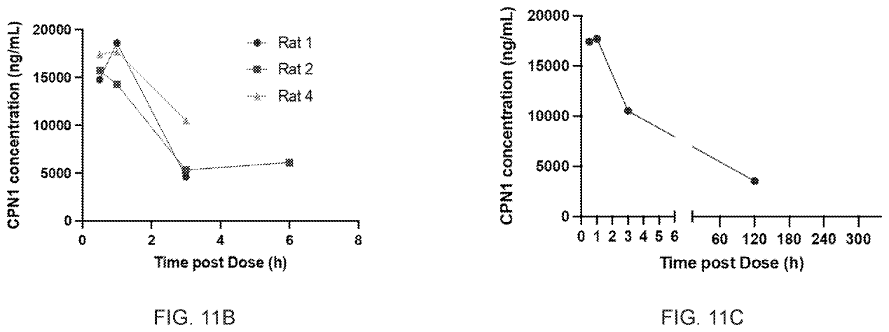

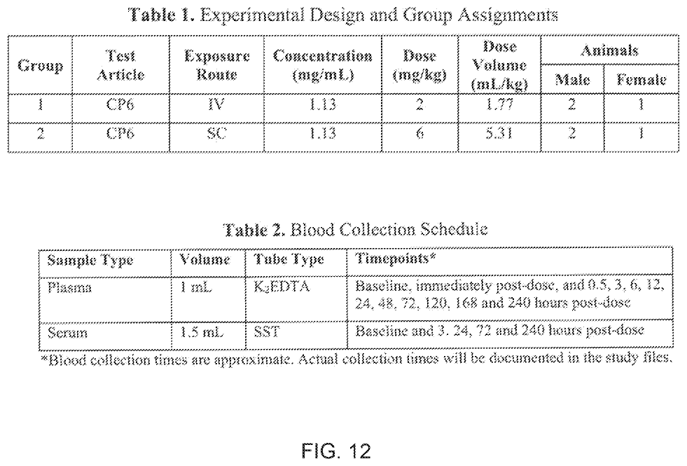

Embodiment I-1. A variant of a carboxypeptidase N (CPN) comprising at least one modification with respect to a wild type CPN, wherein the variant has at least one improved characteristic as compared to the wild type CPN.
Embodiment I-2. The variant of embodiment I-1, wherein the improved characteristic is selected from an increase or a decrease in any one or more of: half-life, activity, potency, affinity for one or more substrates, sensitivity, cofactor affinity, catalytic capability, and selectivity.
Embodiment I-3. The variant of embodiment I-2, wherein the at least one improved characteristic comprises an increase in affinity for one or more substrates, and wherein at least one substrate is C3a.
Embodiment I-4. The variant of any one of embodiments I-2 to I-3, wherein the at least one improved characteristic comprises an increase in affinity for one or more substrates, and wherein at least one substrate is C5a.
Embodiment I-5. The variant of any one of embodiments I-2 to I-4, wherein an increase in activity comprises an increase in the cleavage of C3a and/or C5a.
Embodiment I-6. The variant of any one of embodiments I-2 to I-5, wherein the increase in sensitivity comprises an increase in the sensitivity to any one or more of: MASP1, MASP3, Factor D, mast cell degranulation, mCPA3, and neutrophil degranulation cathepsin G.
Embodiment I-7. The variant of any one of embodiments I-2 to I-6, wherein the increase in activity comprises an increased k_{cat}/K_{M} (M⁻¹ s⁻¹) for cleavage of C3a and/or C5a.
Embodiment I-8. The variant of embodiment I-7, wherein the increased k_{cat}/K_{M} (M⁻¹ s⁻¹) for cleavage of C3a and/or C5a is about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, or about 100-fold greater than that of the wild type CPN.
Embodiment I-9. The variant of embodiment I-7, wherein the increase in activity comprises an increase in k_{cat} with a decrease in K_{M}.
Embodiment I-10. The variant of any one of embodiments I-1 to I-9, wherein the increased activity comprises a decreased K_{D} (nM) value for cleavage of C3a and/or C5a.
Embodiment I-11. The variant of embodiment I-10, wherein the decreased K_{D} (nM) value is about 2-fold, about 3-fold, about 4-fold, about 5-fold, about 6-fold, about 7-fold, about 8-fold, about 9-fold, about 10-fold less than that of the wild type CPN.
Embodiment I-12. The variant of any one of embodiments I-2 to I-11, wherein the increased activity comprises a decreased EC₅₀ (nM) value for cleavage of C3a and/or C5a, compared to the wild type CPN.
Embodiment I-13. The variant of embodiment I-12, wherein the decreased EC₅₀ (nM) value for cleavage of C3a and/or C5a is about 10, about 15, about 20, or less than about 20.
Embodiment I-14. The variant of any one of embodiments I-2 to I-13, wherein the increased half-life is half-life in plasma.
Embodiment I-15. The variant of embodiment I-14, wherein the increased half-life in plasma is greater than about 24 hours.
Embodiment I-16. The variant of any one of embodiments I-2 to I-15, wherein the increased half-life in plasma is from about 70 hours to about 150 hours.
Embodiment I-17. The variant of any one of embodiments I-2 to I-16, wherein the increased sensitivity comprises increased sensitivity for complement activation.
Embodiment I-18. The variant of any one of embodiments I-1 to I-17, wherein the variant comprises at least one modification corresponding to a wild type non-human CPN.
Embodiment I-19. The variant of any one of embodiments I-1 to I-17, wherein the variant comprises at least one modification corresponding to a wild type human CPN.
Embodiment I-20. The variant of embodiment I-19, wherein the CPN variant comprises at least one modification corresponding to a wild type CPN comprising the amino acid sequence as set forth in SEQ ID NO: 1.
Embodiment I-21. The variant of any one of embodiments I-1 to I-20, comprising one or more of domains of a wild type CPN selected from: a first peptide signal, a catalytic domain (CPN1), a second peptide signal, and a regulatory subunit (CPN2).
Embodiment I-22. The variant of any one of embodiments I-1 to I-21, wherein the modification with respect to a wild type CPN comprises any one or more of: a deletion of one or more amino acid residues, a deletion of one or more CPN domains, a substitution of one or more amino acid residues, an insertion of one or more amino acid residues, an insertion of one or more CPN domains, a swapping of one or more CPN domains, and an insertion of at least one non-CPN domain or component.
Embodiment I-23. The variant of embodiment I-22, wherein the at least one non-CPN domain or component comprises at least one domain of any one or more of: carboxypeptidase B2 (CPB2), carboxypeptidase A4 (CPA4), and carboxypeptidase A1 (CPA1).
Embodiment I-24. The variant of embodiment I-23, wherein the at least one non-CPN domain is at least one domain of CPB2 comprising an activation peptide.
Embodiment I-25. The variant of embodiment I-23, wherein the at least one non-CPN domain is at least one domain of CPA4 comprising an activation peptide.
Embodiment I-26. The variant of embodiment I-23, wherein the at least one non-CPN domain is at least one domain of CPA1 comprising an activation peptide.
Embodiment I-27. The variant of any one of embodiments I-24 to I-26, wherein the activation peptide increases sensitivity of the CPN variant for any one or more of: thrombin-thrombomodulin, MASP1, MASP3, Factor D, mCPA3, cathepsin G.
Embodiment I-28. The variant of any one of embodiments I-24 to I-27, wherein the activation peptide increases sensitivity of the CPN variant for any one or more of: mast cell degranulation, neutrophil degranulation, and inflammatory cell activation.
Embodiment I-29. The variant of any one of embodiments I-24 to I-28, wherein the activation peptide increases sensitivity of the CPN variant to complement activation.
Embodiment I-30. The variant of any one of embodiments I-1 to I-29, wherein the deletion of one or more CPN domains comprises deletion of a CPN2 domain.
Embodiment I-31. The variant of any one of embodiments I-23 to I-29, wherein the at least one CPB2 domain comprises a CPB2 catalytic domain.
Embodiment I-32. The variant of any one of embodiments I-23 to I-31, wherein the CPN variant comprises a structural arrangement from N-terminus to C-terminus as (first peptide signal)-(CPB2 activation peptide)-(CPN2).
Embodiment I-33. The variant of any one of embodiments I-1 to I-32, wherein the CPN variant is further fused to a second component.
Embodiment I-34. The variant of embodiment I-33, wherein second component comprises a half-life extender.
Embodiment I-35. The variant of embodiment I-34, wherein the half-life extender is selected from the group consisting of: PEGylation, PASylation, carbohydrates, albumin, and Fc.
Embodiment I-36. The variant of any one of embodiments I-22 to I-35, wherein the insertion of at least one non-CPN domain or component comprises a first non-CPN domain or component a second non-CPN domain or component.
Embodiment I-37. The variant of embodiment I-36, wherein the CPN variant comprises a structural arrangement from N-terminus to C-terminus as (first peptide signal)-(first non-CPN domain or component)-(CPN2)-(second non-CPN domain or component).
Embodiment I-38. The variant of embodiment I-37, wherein the first non-CPN domain or component is an activation peptide or a half-life extender.
Embodiment I-39. The variant of any one of embodiments I-37 to I-38, wherein the second non-CPN domain or component is a half-life extender or activation peptide.
Embodiment I-40. The variant of any one of embodiments I-1 to I-39, wherein the variant is a tetramer comprising two heterodimers.
Embodiment I-41. The variant of any one of embodiments I-1 to I-39, wherein the variant is a single heterodimer.
Embodiment I-42. The variant of any one of embodiments I-1 to I-41, wherein the variant is non-immunogenic.
Embodiment I-43. The variant of any one of embodiments I-1 to I-42, wherein the variant is in a zymogen form.
Embodiment I-44. The variant of any one of embodiments I-1 to I-42, wherein the variant is in an active form.
Embodiment I-45. A method of treating a disease or condition in a subject in need thereof, comprising administering to the subject any one of the variants of embodiments I-1 to I-44.
Embodiment I-46. The method of embodiment I-45, wherein the disease or condition is selected from the group consisting of: congenital complement deficiency, control protein deficiency, secondary complement disorder, immunity related disorder, chronic renal disorder, acute inflammatory disorder, antineutrophil cytoplasmic antibody (ANCA)-associated vasculitis (AAV), C3 glomerulopathy (C3G), lupus nephritis, skin disorder, intestinal ischemia and reperfusion (I/R) injury, sepsis, mast cell related disorders, and solid tumors refractory to immunotherapy agents such as pembrolizumab and ipilimumab.
Embodiment I-47. The method of embodiment I-45, wherein the disease or condition is an acute condition selected from the group consisting of: acute respiratory distress syndrome (ARDS), COVID-19, multisystem organ failure, and sepsis.
Embodiment I-48. The method of embodiment I-45, wherein the disease or condition is a chronic condition selected from the group consisting of: anti-neutrophil cytoplasmic autoantibody (ANCA) vasculitis, atypical hemolytic uremic syndrome (aHUS), and IgA nephropathy.
Embodiment I-49. The method of embodiment I-46, wherein the disease or condition is a skin disorder selected from the group consisting of: hidradenitis suppurativa (HS), bullous pemphigoid (BP), and Pyoderma Gangrenosum.
Embodiment I-50. The method of any one of embodiments I-45 to I-49, wherein the variant is administered in a zymogen form.
Embodiment I-51. The method of any one of embodiments I-45 to I-49, wherein the variant is administered in an active form.
Embodiment I-52. The method of any one of embodiments I-45 to I-49, wherein the administration is a subcutaneous administration.
Embodiment I-53. The method of any one of embodiments I-45 to I-49, wherein the administration is an intravenous administration.
Embodiment I-54. The method of any one of embodiments I-45 to I-49, wherein the administration comprises administering a vector comprising a nucleic acid encoding any one of the variants of embodiments I-1 to I-44.
Embodiment I-55. A nucleic acid encoding any one of the variants of embodiments I-1 to I-44.
Embodiment I-56. A pharmaceutical composition comprising any one of the variants of embodiments I-1 to I-44, and optionally a pharmaceutically acceptable carrier.
Embodiment I-57. A variant of a carboxypeptidase N1 (CPN1) comprising at least one modification with respect to a wild type CPN1, wherein the variant has at least one improved characteristic as compared to the wild type CPN1.
Embodiment I-58. A CPN1 variant selected from FIG. 11 of U.S. Ser. No. 63/222,929.
Embodiment I-59. A CPN1 construct selected from FIG. 12 of U.S. Ser. No. 63/222,929.
Embodiment I-60. A CPN1 construct comprising a CPN1 variant of FIG. 11 of U.S. Ser. No. 63/222,929.

### Set II

Embodiment II-1. A variant of a carboxypeptidase N catalytic subunit (CPN1) comprising at least one modification with respect to a wild type CPN1, wherein the variant has at least one improved characteristic as compared to the wild type CPN1.
Embodiment II-2. The variant of embodiment II-1, wherein the modification with respect to a wild type CPN1 comprises any one or more of: a substitution of one or more amino acid residues, a deletion of one or more amino acid residues, an insertion of one or more amino acid residues, an insertion of one or more CPN domains, and an insertion of one ore more non-CPN domains or components.
Embodiment II-3. The variant of any one of embodiments II-1 to II-2, wherein the improved characteristic is selected from an increase or a decrease in any one or more of: half-life, activity, potency, substrate affinity, substrate specificity, substrate selectivity, proteolytic sensitivity, cofactor affinity, and catalytic capability.
Embodiment II-4. The variant of embodiment II-3, wherein the at least one improved characteristic comprises an increase in affinity for one or more substrates, and wherein at least one substrate is C3a.
Embodiment II-5. The variant of any one of embodiments II-3 to II-4, wherein the at least one improved characteristic comprises an increase in affinity for one or more substrates, and wherein at least one substrate is C5a.
Embodiment II-6. The variant of any one of embodiments II-3 to II-5, wherein an increase in activity comprises an increase in the cleavage of C3a and/or C5a.
Embodiment II-7. The variant of any one of embodiments II-3 to II-6, wherein the increase in activity comprises an increased k_{cat}/K_{M} (M⁻¹ s⁻¹) for cleavage of C3a and/or C5a.
Embodiment II-8. The variant of embodiment II-7, wherein the increased k_{cat}/K_{M} (M⁻¹ s⁻¹) for cleavage of C3a and/or C5a is about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, or about 100-fold greater than that of the wild type CPN.
Embodiment II-9. The variant of embodiment II-7, wherein the increase in activity comprises an increase in k_{cat} with a decrease in K_{M}.
Embodiment II-10. The variant of any one of embodiments II-1 to II-9, wherein the increased activity comprises a decreased K_{D} (nM) value for cleavage of C3a and/or C5a.
Embodiment II-11. The variant of embodiment II-10, wherein the decreased K_{D} (nM) value is about 2-fold, about 3-fold, about 4-fold, about 5-fold, about 6-fold, about 7-fold, about 8-fold, about 9-fold, about 10-fold less than that of the wild type CPN.
Embodiment II-12. The variant of any one of embodiments II-3 to II-11, wherein the increased activity comprises a decreased EC₅₀ (nM) value for cleavage of C3a and/or C5a, compared to the wild type CPN.
Embodiment II-13. The variant of embodiment II-12, wherein the decreased EC₅₀ (nM) value for cleavage of C3a and/or C5a is about 10, about 15, about 20, or less than about 20.
Embodiment II-14. The variant of any one of embodiments II-3 to II-13, wherein the increased half-life is observed in plasma.
Embodiment II-15. The variant of embodiment II-14, wherein the increased half-life in plasma is greater than about 24 hours.
Embodiment II-16. The variant of any one of embodiments II-3 to II-15, wherein the increased half-life in plasma is from about 70 hours to about 150 hours.
Embodiment II-17. The variant of any one of embodiments II-1 to II-16, wherein the variant comprises at least one modification corresponding to a wild type non-human CPN1.
Embodiment II-18. The variant of any one of embodiments II-1 to II-16, wherein the variant comprises at least one modification corresponding to a wild type human CPN1.
Embodiment II-19. The variant of embodiment II-18, wherein the CPN1 variant comprises at least one modification corresponding to a wild type CPN1 comprising the amino acid sequence as set forth in SEQ ID NO: 6.
Embodiment II-20. The variant of any one of embodiments II-1 to II-19, wherein the CPN1 variant comprises an amino acid having at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 6.
Embodiment II-21. The variant of any one of embodiments II-1 to II-19, wherein the CPN1 variant comprises the amino acid sequence as set forth in SEQ ID NO: 7, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-22. The variant of any one of embodiments II-1 to II-19, wherein the CPN1 variant comprises the amino acid sequence as set forth in SEQ ID NO: 8, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-23. The variant of any one of embodiments II-1 to II-19, wherein the CPN1 variant comprises the amino acid sequence as set forth in SEQ ID NO: 11, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-24. The variant of any one of embodiments II-1 to II-19, wherein the CPN1 variant comprises the amino acid sequence as set forth in SEQ ID NO: 12, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-25. The variant of any one of embodiments II-1 to II-19, wherein the CPN1 variant comprises SEQ ID NO: 6, SEQ ID NO: 7, SEQ ID NO: 8, SEQ ID NO: 11, or SEQ ID NO:12, comprising one of the modification strings selected from the group consisting of the modification strings provided in Table 4A, Table 4B, and Table 4C.
Embodiment II-26. The variant of any one of embodiments II-1 to II-19, wherein the CPN1 variant comprises SEQ ID NO: 6, SEQ ID NO: 7, SEQ ID NO: 8, SEQ ID NO: 11, or SEQ ID NO:12, comprising one of the modification strings selected from the group consisting of the modification strings provided in Table 3A, Table 3B, and Table 3C.
Embodiment II-27. A fusion construct comprising a carboxypeptidase N catalytic subunit (CPN1) or variant thereof.
Embodiment II-28. The fusion construct of embodiment II-27, selected from the group consisting of SEQ ID NO: 73-SEQ ID NO: 485.
Embodiment II-29. The fusion construct of embodiment II-27, wherein the construct comprises a CPN1 of SEQ ID NO: 6.
Embodiment II-30. The fusion construct of embodiment II-27, wherein the CPN1 variant comprises an amino acid having at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to SEQ ID NO: 6.
Embodiment II-31. The fusion construct of embodiment II-27, wherein the CPN1 variant is selected from any one of the variants of embodiments II-1 to II-26.
Embodiment II-32. The fusion construct of embodiment II-27, wherein the CPN1 variant comprises at least one modification corresponding to a wild type CPN1 comprising the amino acid sequence as set forth in SEQ ID NO: 6.
Embodiment II-33. The fusion construct of embodiment II-27, wherein the CPN1 variant comprises the amino acid sequence as set forth in SEQ ID NO: 7, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-34. The fusion construct of embodiment II-27, wherein the CPN1 variant comprises the amino acid sequence as set forth in SEQ ID NO: 8, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-35. The fusion construct of embodiment II-27, wherein the CPN1 variant comprises the amino acid sequence as set forth in SEQ ID NO: 11, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-36. The fusion construct of embodiment II-27, wherein the CPN1 variant comprises the amino acid sequence as set forth in SEQ ID NO: 12, or at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity thereto.
Embodiment II-37. The fusion construct of embodiment II-27, wherein the CPN1 variant comprises SEQ ID NO: 6, SEQ ID NO: 7, SEQ ID NO: 8, SEQ ID NO: 11, or SEQ ID NO:12, comprising one or more modifications selected from the group consisting of the modifications provided in Table 4A, Table 4B, and Table 4C.
Embodiment II-38. The fusion construct of embodiment II-27, wherein the CPN1 variant comprises SEQ ID NO: 6, SEQ ID NO: 7, SEQ ID NO: 8, SEQ ID NO: 11, or SEQ ID NO:12, comprising one of the modification strings selected from the group consisting of the modification strings provided in Table 3A, Table 3B, and Table 3C.
Embodiment II-39. The fusion construct of any one of embodiments II-27 to II-38, wherein the fusion construct comprises a Glutathione S transferase (GST) amino acid sequence.
Embodiment II-40. The fusion construct of embodiment II-39, wherein the GST amino acid sequence comprises SEQ ID NO: 31.
Embodiment II-41. The fusion construct of any one of embodiments II-27 to II-40, wherein the fusion construct comprises a mammalian maltose binding protein (mMBP) amino acid sequence.
Embodiment II-42. The fusion construct of embodiment II-41, wherein the mMBP amino acid sequence comprises SEQ ID NO: 30.
Embodiment II-43. The fusion construct of any one of embodiments II-27 to II-42, wherein the fusion construct comprises a small ubiquitin modifying enzyme (SUMO) amino acid sequence.
Embodiment II-44. The fusion construct of embodiment II-43, wherein the SUMO amino acid sequence comprises SEQ ID NO: 23 or SEQ ID NO: 24.
Embodiment II-45. The fusion construct of any one of embodiments II-27 to II-44, wherein the fusion construct comprises a Tobacco Etch Virus protease cleavage site (TEV) amino acid sequence.
Embodiment II-46. The fusion construct of embodiment II-45, wherein the TEV amino acid sequence comprises SEQ ID NO: 25.
Embodiment II-47. The fusion construct of any one of embodiments II-27 to II-46, wherein the fusion construct comprises an activation peptide of CBP2 (the N-terminal 96aa of a CBP2 protease) amino acid sequence.
Embodiment II-48. The fusion construct of embodiment II-47, wherein the amino acid sequence of the activation peptide of CBP2 comprises SEQ ID NO: 22.
Embodiment II-49. The fusion construct of any one of embodiments II-27 to II-48, wherein the fusion construct comprises an Factor Xa protease cleavage site (Xa) amino acid sequence.
Embodiment II-50. The fusion construct of embodiment II-49, wherein the Factor Xa protease cleavage site (Xa) comprises the amino acid sequence of SEQ ID NO: 29.
Embodiment II-51. The fusion construct of any one of embodiments II-27 to II-50, wherein the fusion construct comprises a portion of a regulatory CPN2 subunit amino acid sequence.
Embodiment II-52. The fusion construct of embodiment II-51, wherein the CPN2 amino acid sequence is selected from the group consisting of: CPN2 (1-367), CPN2 (1-370), CPN2 (1-425), CPN2 (1-456), and CPN2 (1-524).
Embodiment II-53. The fusion construct of any one of embodiments II-27 to II-52, wherein the fusion construct comprises an CD180 amino acid sequence.
Embodiment II-54. The fusion construct of any one of embodiments II-27 to II-53, wherein the fusion construct comprises an CD180 amino acid sequence.
Embodiment II-55. The fusion construct of any one of embodiments II-27 to II-54, wherein the fusion construct comprises LR1G1 amino acid sequence.
Embodiment II-56. The fusion construct of any one of embodiments II-27 to II-38, wherein the fusion construct comprises at least one non-CPN1 or non-CPN2 domain or component.
Embodiment II-57. The fusion construct of embodiment II-56, wherein the at least one non-CPN1 or non-CPN2 domain or component comprises at least one domain of any one or more of: carboxypeptidase B2 (CPB2), carboxypeptidase A4 (CPA4), and carboxypeptidase A1 (CPA1).
Embodiment II-58. The fusion construct of any one of embodiments II-27 to II-57, wherein the fusion construct comprises an activation peptide that increases sensitivity of the fusion construct.
Embodiment II-59. The fusion construct of embodiment II-58, wherein the construct comprises an activation peptide that increases sensitivity of the fusion construct for any one or more of: mast cell degranulation, neutrophil degranulation, and inflammatory cell activation.
Embodiment II-60. The fusion construct of embodiment II-58, wherein the activation peptide increases sensitivity of the fusion construct for any one or more of: thrombin-thrombomodulin, MASP1, MASP3, Factor D, mCPA3, complement activation, and cathepsin G.
Embodiment II-61. The fusion construct of any one of embodiments II-27 to II-60, wherein the fusion construct comprises a half-life extender.
Embodiment II-62. The fusion construct of embodiment II-61, wherein the half-life extender is selected from the group consisting of: PEG, PAS, carbohydrates, albumin, and Fc.
Embodiment II-63. The fusion construct of embodiment II-62, wherein the albumin comprises human serum albumin.
Embodiment II-64. The fusion construct of any one of embodiments II-27 to II-63, wherein the fusion construct is non-immunogenic.
Embodiment II-65. The fusion construct of any one of embodiments II-27 to II-64, wherein the fusion construct is in a zymogen form.
Embodiment II-66. The fusion construct of any one of embodiments II-27 to II-65, wherein the fusion construct is in an active form.
Embodiment II-67. A method of treating a disease or condition in a subject in need thereof, comprising administering to the subject any one of the CPN1 variants or fusion constructs of embodiments II-1 to II-66.
Embodiment II-68. The method of embodiment II-67, wherein the disease or condition is selected from the group consisting of: congenital complement deficiency, control protein deficiency, secondary complement disorder, immunity related disorder, chronic renal disorder, acute inflammatory disorder, antineutrophil cytoplasmic antibody (ANCA)-associated vasculitis (AAV), C3 glomerulopathy (C3G), lupus nephritis, skin disorder, intestinal ischemia and reperfusion (I/R) injury, sepsis, mast cell related disorders, and solid tumors refractory to immunotherapy agents such as pembrolizumab and ipilimumab.
Embodiment II-69. The method of embodiment II-67, wherein the disease or condition is an acute condition selected from the group consisting of: acute respiratory distress syndrome (ARDS), COVID-19, multisystem organ failure, and sepsis.
Embodiment II-70. The method of embodiment II-67, wherein the disease or condition is a chronic condition selected from the group consisting of: anti-neutrophil cytoplasmic autoantibody (ANCA) vasculitis, atypical hemolytic uremic syndrome (aHUS), and IgA nephropathy.
Embodiment II-71. The method of embodiment II-68, wherein the disease or condition is a skin disorder selected from the group consisting of: hidradenitis suppurativa (HS), bullous pemphigoid (BP), and Pyoderma Gangrenosum.
Embodiment II-72. The method of any one of embodiments II-67 to II-71, wherein the fusion construct as administered is in a zymogen form.
Embodiment II-73. The method of any one of embodiments II-67 to II-71, wherein the CPN1 variant fusion construct as administered is in an active form.
Embodiment II-74. The method of any one of embodiments II-67 to II-71, wherein the administration of the CPN1 variant or fusion construct is a subcutaneous administration.
Embodiment II-75. The method of any one of embodiments II-67 to II-71, wherein the administration of the CPN1 variant or fusion construct is an intravenous administration.
Embodiment II-76. The method of any one of embodiments II-67 to II-71, wherein the administration comprises administering a vector comprising a nucleic acid encoding any one of the CPN1 variants or fusion constructs of embodiments II-1 to II-66.
Embodiment II-77. A nucleic acid encoding any one of the CPN1 variants or fusion constructs of embodiments II-1 to II-66.
Embodiment II-78. A pharmaceutical composition comprising any one of the CPN1 variants or fusion constructs of embodiments II-1 to II-66, and optionally a pharmaceutically acceptable carrier.

### EXAMPLES

### Example 1: Activation Peptide Screening for Fusion With CPN

A peptide library can be used for the selection of effective activation peptide sequences, which can be expressed as an 8-mer, 10-mer, or a 12-mer, and so on. Purified MASP1, MASP3, Factor D, mCPA3, and cathepsin G can be used to screen for the activation peptide sequences. A mass spectrometry-based method can be used for each protease to evaluate the effectiveness of each activation peptide sequence tested.

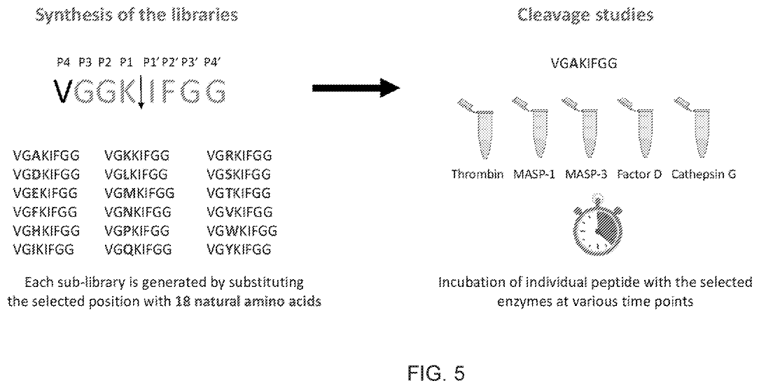

In order to identify sequences efficiently cleaved by a selected enzyme, the positional scanning approach is used. The linear 8-mer (i.e., 8-amino acid long) peptide libraries covering the P4 to P4′ positions are generated for each enzyme based on the sequence of the specific substrates described in the literature. For example, the library for MASP-1 will be based on the cleavage sequence of protease-activated receptor 4 (YPGK↓F, SEQ ID NO: 67)⁵, for MASP-3 on sequence derived of the combinatorial peptide library (GGK↓IFGG, SEQ ID NO: 68)⁶, for Factor D on the cleavage sequence of Factor B (QQKR↓KIVL, SEQ ID NO: 69)⁷, and for cathepsin G on the cleavage sequence of interleukin-33 (VECF↓AFGI, SEQ ID NO: 70)⁸. The selected sequence will be incorporated into the thrombin cleavage site from CPB2 activation peptide (i.e., VSPR↓ASAS, SEQ ID NO: 71). For each library, the screening for the best substitutions (residues with the highest affinity towards the selected enzyme and selectivity over others) will be performed as follows: each of the selected position (with the exception of the positions indicated in red which are necessary for enzyme recognition) will be replaced, one by one, with natural amino acid residues (except for Cys) to generate 18 individual peptides per one position screened. For instance, the library for MASP-3 will consist of 7 sub-libraries for the following positions: P4, P3, P2, P1, P1′, P2′, P3′, P4′, wherein the positions P are counted from the point of cleavage, which is between P1 and P′, each containing 18 individual peptides (total of 126 peptides). Subsequently, the cleavage studies are performed by incubating the obtained analogs with the selected enzymes at different time points (at least 6 time points). FIG. 5 depicts a general schematic diagram of the screening process, and examples of libraries for the activation peptide screening.

### Methodology

Peptides are prepared by manual and automated solid-phase peptide synthesis based on standard Fmoc strategy protocols using 2-chlorotrityl chloride resin. After completion of the synthesis, peptides are cleaved using trifluoroacetic acid (TFA) and required scavengers (selected based on the peptide sequence). The obtained crudes are purified using high performance liquid chromatography (HPLC) and their purity (>95%) will be characterized by HPLC. Enzymes will be purchased from commercial suppliers (i.e., R&D Systems, Enzo Life Science). The conditions (e.g., enzyme and peptide concentrations, pH) for the cleavage studies will be optimized for each enzyme using commercially available fluorogenic substrates. The cleavage experiments will be performed by incubating each peptide with the selected enzyme at different time points (0, 5, 15, 30, 60, 120 and 240 min). The reactions will be quenched with formic acid. The samples will be centrifuged, supernatant will be collected recovered, and stored in −80° C. Cleavage analysis will be done by HPLC and mass spectrometry analysis.

### Example 2: Screening for Activated CPN Variants

Assays are performed to evaluate the activation of a CPN zymogen, followed by C3a and/or C5a digestion, for selection of effective CPN variants. The assays are used to determine the k_{cat}/K_{M} of the activated CPN. A first assay is used to screen the CPN variants' k_{cat}/K_{M} of towards C3a versus C5a, and a second assay is used to screen the zymogen forms of CPN chimeras for their activation by complement enzymes.

Briefly, CPB2, CPN wild type and CPN chimeras are incubated with their respective complement activation enzyme and the negative control T-TM complex for a period of time before the reaction is terminated by the addition of PPACK. Kinetic analysis of hydrolysis of 8-10-mer C3a and C5a peptides by CPB2 and CPN is performed, and Michaelis-Menten kinetics is used to determine the k_{cat}/K_{M} for the hydrolysis by CPB2 or CPN of the 10-mer peptide. The portion of the C3a and C5a peptides used for the assay are indicated in bold in Table 2 below.

TABLE 2.1
C3a and C5a Peptide Substrate Sequences

| | |
|---|---|
| C3a | SVQLTEKRMDKVGKYPKELRKCCEDGMRENPMRFSCQ |
| | RRTRFISLGEACKKVELDCCNYITELRRQHARASHLG |
| | LAR |
| | (SEQ ID NO: 4) |
| C5a | TLQKKIEEIAAKYKHSVVKKCCYDGACVNNDETCEQR |
| | AARISLGPRCIKAFTECCVVASQLRANISHKDMQLGR |
| | (SEQ ID NO: 5) |

The cleaved peptide is resolved by HPLC, and the nmol of the peptide generated is determined from the peak area of cleaved peptide. The values for K_{M} and k_{cat} are then determined by plotting the initial velocities of cleavage against the different substrate concentrations tested, and fitting to the Michaelis-Menten equation by non-linear regression, as is known in the art.

### Example: 3: Receptor Activation Assay

A beta-arrestin GPCR assay can be used to measure the activity of the CPN variants for their substrates, which can be C3a and/or C5a. C3a and/or C5a receptor activation upon binding of C3a and/or C5a is measured by the amount of luminescence generated, indicative of recruitment of beta-arrestin, which is directly related to receptor activation, thus providing a determination of the activity level of the C3a and/or C5a. Briefly, CPN variants are incubated with C3a and/or C5a and the digestion is stopped with a CPN inhibitor. Digests are then incubated with cells and detecting reagents are added. Luminescence is read to measure the amount of residual C3a or C5a resulting from the CPN variant digestion.

### Example 4: Functional Assay—Calcium Signaling

A functional assay measuring calcium signaling can be performed to assess the level of C3a or C5a inhibition by CPN variants. Imaging can be done to show the calcium signaling taking place. Briefly, cells transfected with C3aR or C5aR are incubated with the CPN variants to be tested, to determine whether the CPN variants are capable of directly activating C3aR or C5aR, and whether the CPN variants are capable of interfering with C3a or C5a to thus avoid C3aR or C5aR activation. The main readout for cell line activation will be the monitoring of intracellular calcium levels (increased after C3aR or C5aR activation) by real-time fluorescence microscopy in a temperature-controlled atmosphere. Following treatments, intracellular calcium will be visualized with a time-lapse fluorescent microscope. Levels of calcium signaling shown through fluorescent imaging will determine whether the CPN variants are effectively interfering with C3a or C5a to thus prevent or reduce C3aR or C5aR activation. HEK cells expressing or not expressing the receptors C3a and C5a are loaded with Fluo-4, a calcium sensitive probe, then imaged under an Apotome microscope wide field, 20× objective with a temperature-controlled atmosphere. Quantification of fluorescent signals are performed using the software ImageJ, and global increase in mean fluorescence intensity (MFI) is evaluated.

### Example 5: In Vivo Models of Disease

Animal models can be used for further study of diseases related to dysregulation of complement. Chronic models include: lupus nephritis using MRL/lpr and NZB/W F1 mice, C3G using rats, ANCA AAV using mice, bullous pemphigoid using mice. Acute models include: cecal ligation and puncture using rats, peritonitis using mice, LPS lung injury using mice, and kidney transplant I/R injury using mice.

### Example 6: Expression of CPN1 Variants

In general, the Expi293™ Expression System Kit (A14635 From Thermo Fisher Scientific) was used to transfect and transiently express CPN1 and its variants. Briefly, cells were diluted in pre-warmed media to indicated desired starting density in an appropriate vessel size based on volume. Optionally, cells were pre-diluted to desired transfection density for multiple transfections and desired total mL column used. DNA/optimem and Expifectamine/optimem were prepared as two separate mixtures, inverted, and incubated for 5 mins at RT. Optionally, expifectamine/optimem master mix with ˜3% extra volume for multiple transfections can be made. Expifectamine mix was added to DNA mix, mixed by inversion, and allowed to complex for 10 mins. Complexes were added dropwise to cells while swirling. Cells were placed in an incubator at an appropriate shake speed for vessel size.

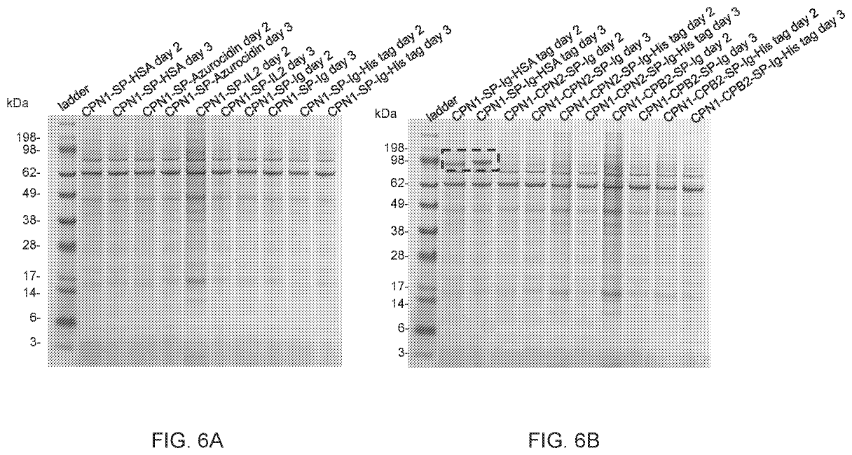

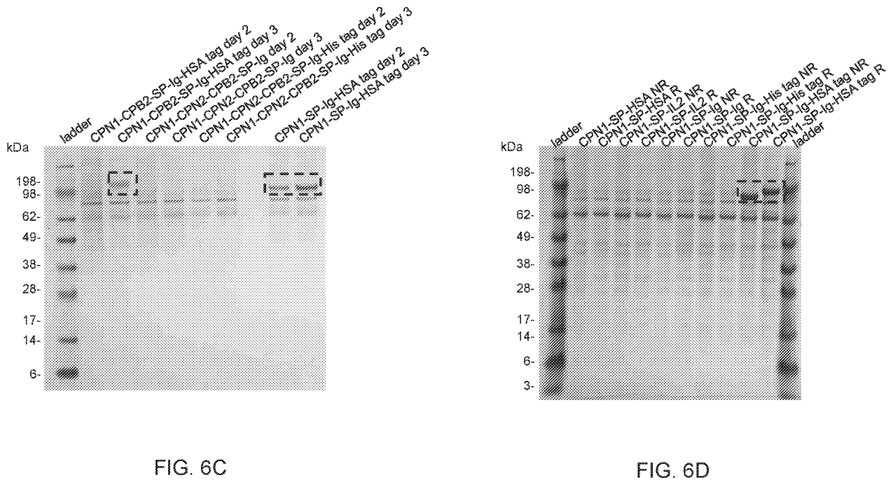

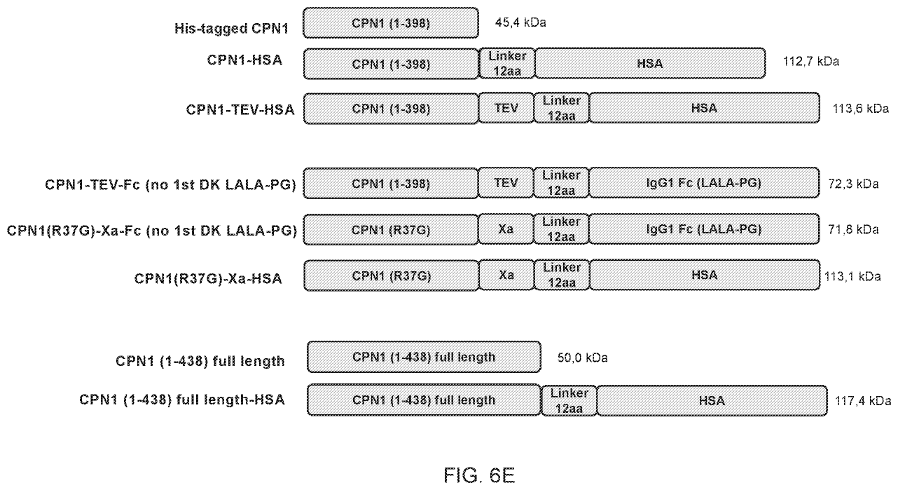

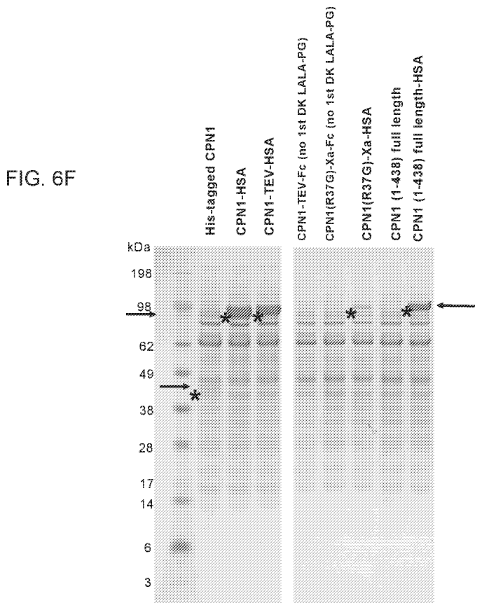

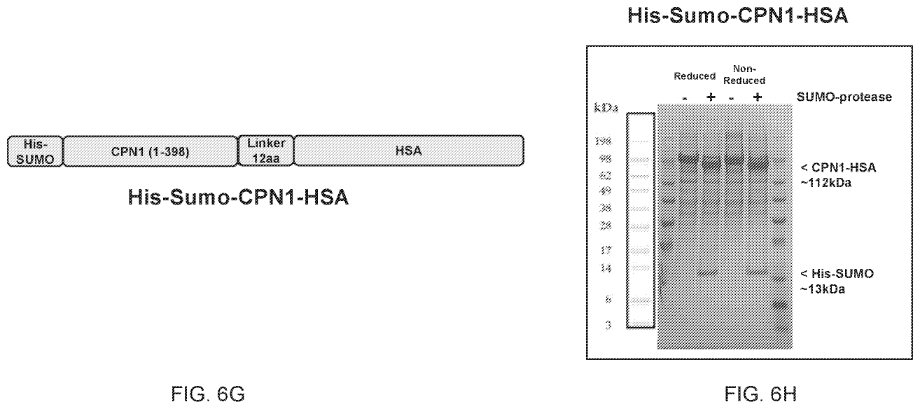

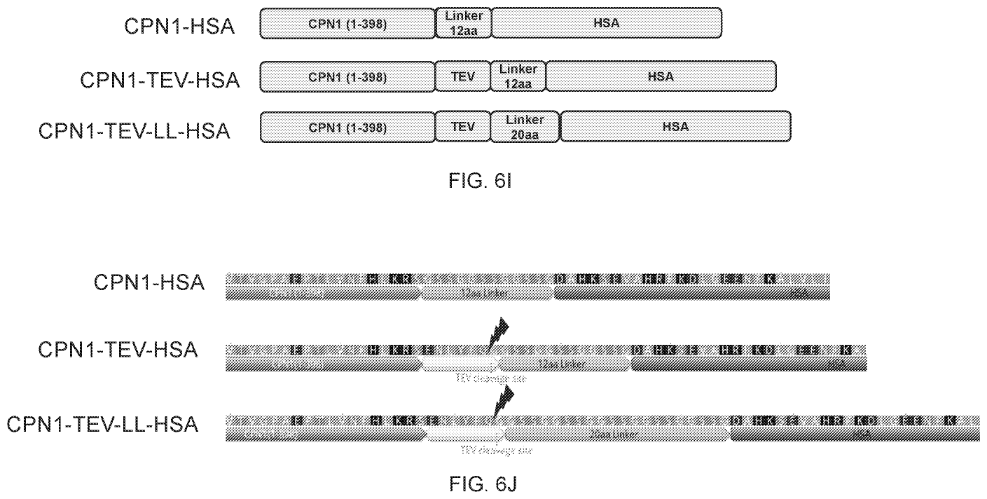

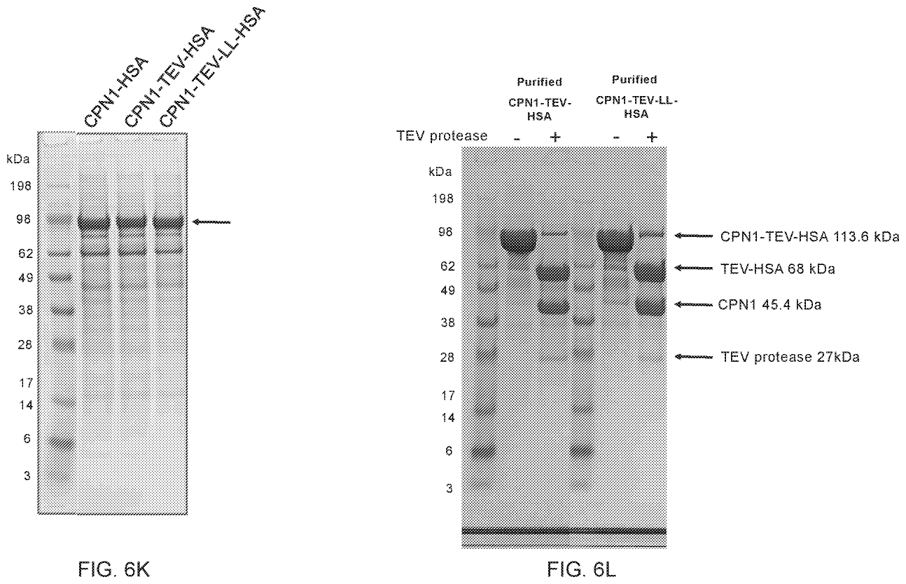

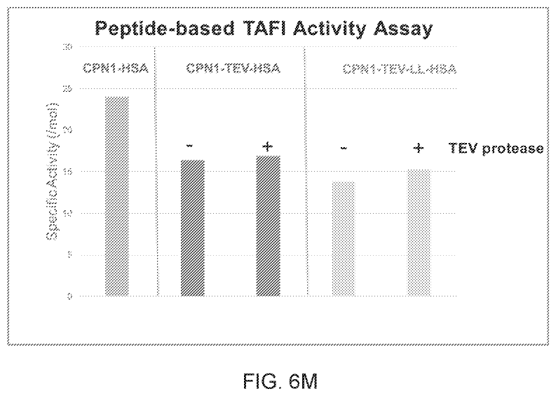

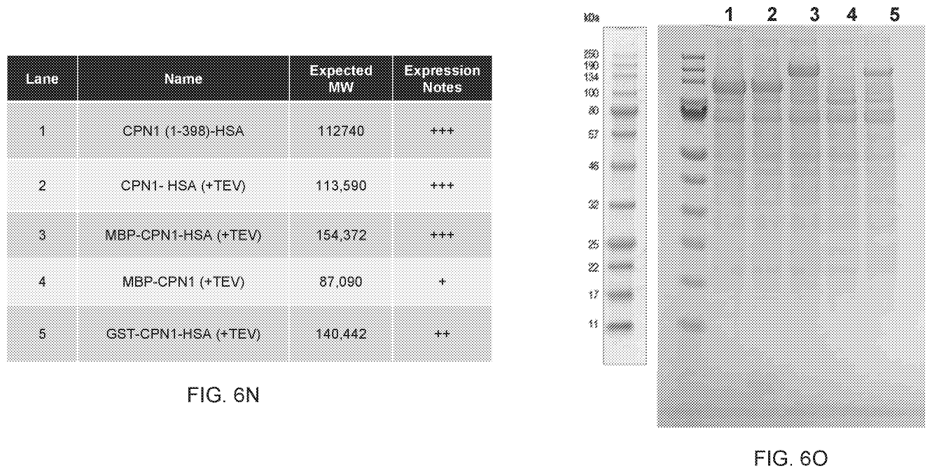

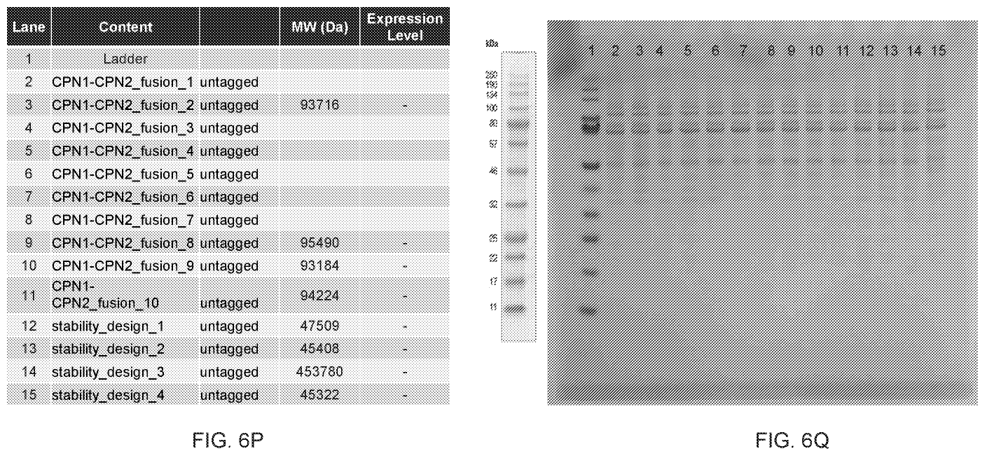

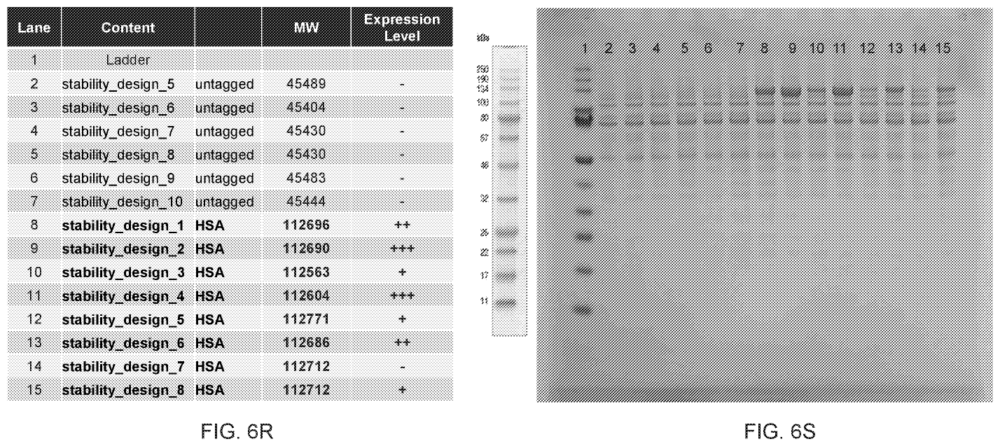

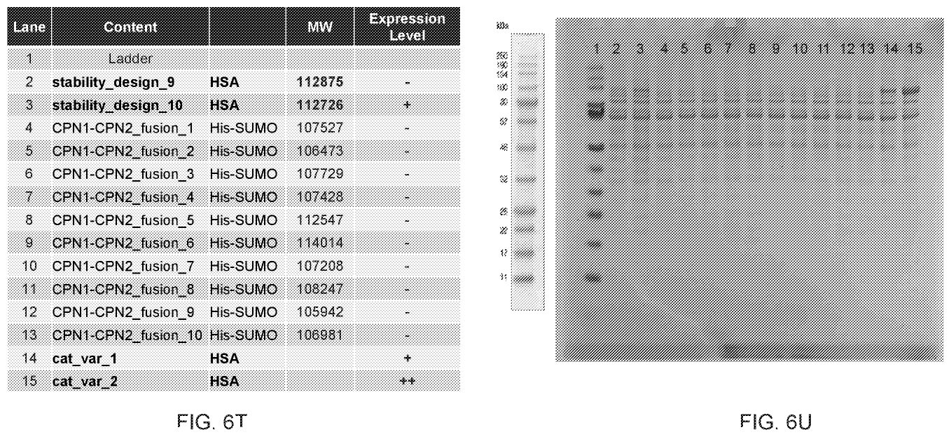

FIGS. 6A-6C depict Coomassie staining from SDS-PAGE analysis showing various CPN chimeras expressed in Expi293 cells. Generally, the expression of CPN1-HSA and CPN1-CPB2-HSA were shown to have better expression in Expi293 cells than the other tested constructs.

Thirteen CPN1 constructs were transiently transfected in Expi293 cells. After 2 or 3 days of transient expression, cell supernatants were collected for SDS PAGE gel analysis. Cell supernatants were mixed with NuPage loading dye containing reducing agent and samples heated 95° C. for 5 minutes. Samples were loaded in Tris-Bis gel 4-12% and run at 150V for 1 hour. Gels were incubated with SimplyBlue Safe stain to detect proteins. Among the 13 constructs tested CPN1-SP-Ig-HSA tag and CPN1-CPB2-SP-Ig-HSA tag expressed best.

FIG. 6D shows a Coomassie staining from SDS-PAGE analysis of CPN1 construct expression, using a similar process as depicted in FIGS. 6A-6C, with five CPN1 constructs. These five CPN1 constructs were also transiently transfected in Expi293 cells. After 3 days of transient expression, cell supernatants were collected for SDS-PAGE gel analysis. Cell supernatants were mixed with NuPage loading dye with (R) or without (NR) reducing agent and samples heated 95° C. for 5 minutes. Samples were loaded in Tris-Bis gel 4-12% and run at 150V for 1 hour. Gels were incubated with SimplyBlue Safe stain to detect proteins. Overall, among these five constructs, CPN1-SP-Ig-HSA tag expressed best.

FIGS. 6E-6F show eight exemplary CPN1 constructs and their corresponding SDS-PAGE analysis of cell culture supernatants after 4 days of transient expression in Expi293 cells. Generally, the constructs with HSA fusion showed the best expression. Similarly, FIG. 6G-6H shows the construct His-SUMO-CPN1-HSA and its SDS-PAGE analysis. The results show that His-SUMO-CPN1-HSA can be expressed and purified by His-Trap and SUMO cleavage.

FIGS. 61-6J shows exemplary CPN1 constructs with a TEV protease cleavage site (CPN1-TEV-HSA and CPN1-TEV-LL-HSA) and without a TEV protease cleavage site (CPN1-HSA). High expression of all three constructs was seen in SDS-PAGE analysis of cell culture supernatants after 4 days of transient expression in Expi293 (FIG. 6K). Further, FIG. 6L shows SDS-PAGE analysis of purified CPN1-TEV-HSA and CPN1-TEV-LL-HSA proteins treated with or without TEV protease. The cleavage of the constructs into CPN1 and TEV-HSA components in samples exposed to TEV protease is clearly seen. Further, to test the CPN1 catalytic activity of CPN1-TEV-HSA and CPN1-TEV-LL-HSA after HSA cleavage by TEV protease, a peptide based TAFI activity assay (Pefakit®) TAFI, Catalogue Number: 800186) was carried out. CPN1-HSA, which has no TEV cleavage site, served as control. Results of the TAFI activity assay are shown in FIG. 6M, which show that CPN1 preserves its catalytic activity after HSA cleavage in both CPN1-TEV-HSA and CPN1-TEV-LL-HSA constructs. Further, FIGS. 6N-60 show additional five exemplary CPN1 constructs and their corresponding SDS-PAGE expression analysis.

FIGS. 6P-6W show the SDS-PAGE expression analysis of further exemplary CPN1 constructs. Samples for the gels were generally prepared with 10 μL 4×LDS/DTT and 30 μL sample. The samples were heated to 90° C. for 5 minutes. 8 μL ladder loaded and 15 μL sample were loaded in relevant lanes. 4-12% Bis-Tris Gels were used and run at 150 V for 50 min. SimplyBlue Safe Stain was used to detect proteins.

### Example 7A: Purification of CPN1 Variants by a 2-Step Purification Process

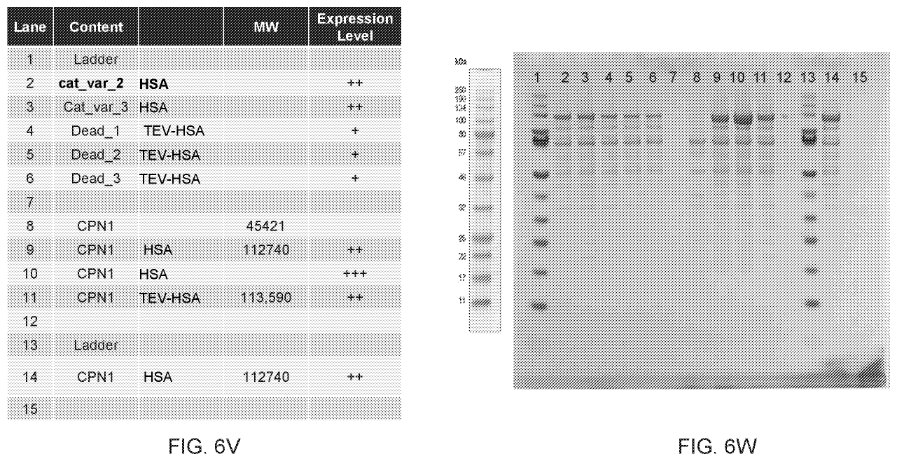

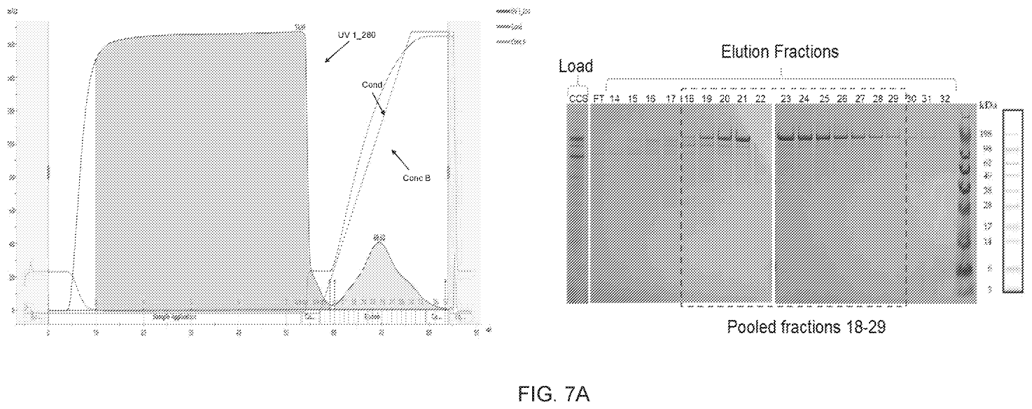

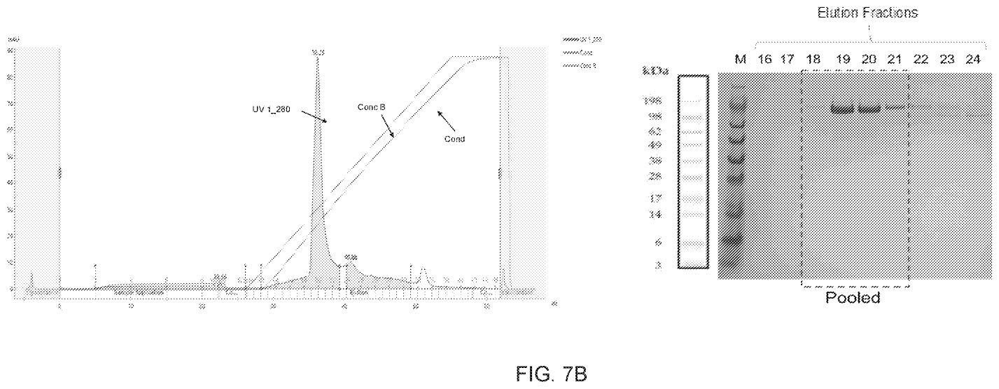

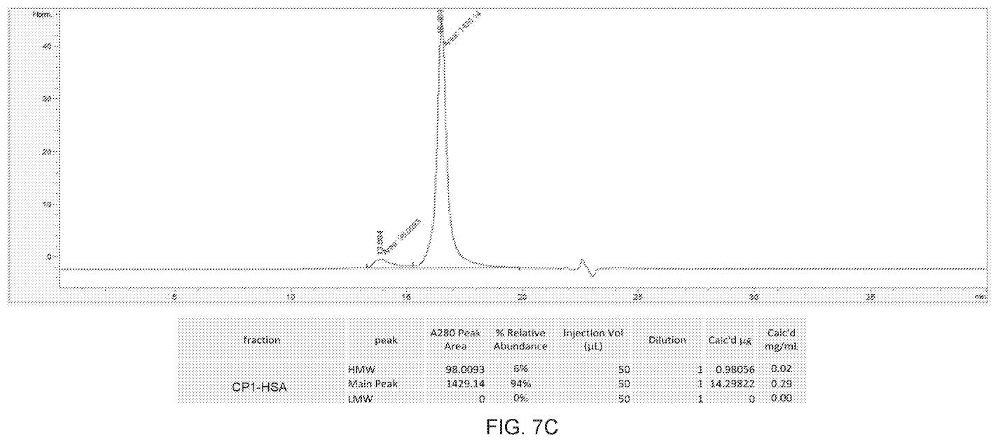

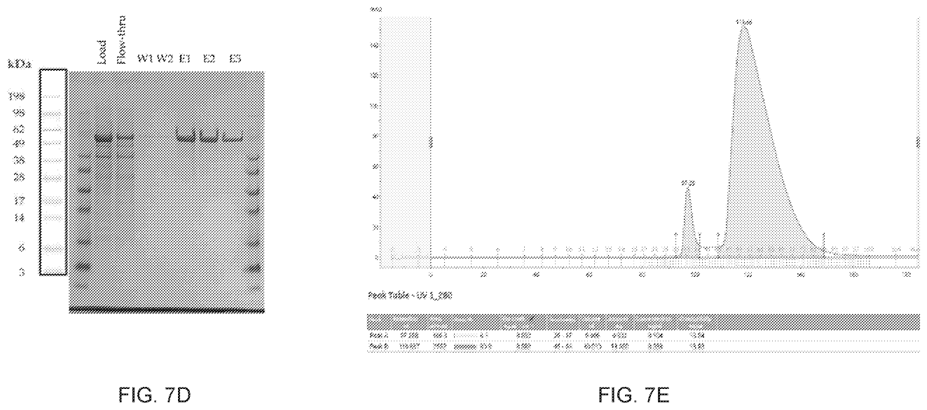

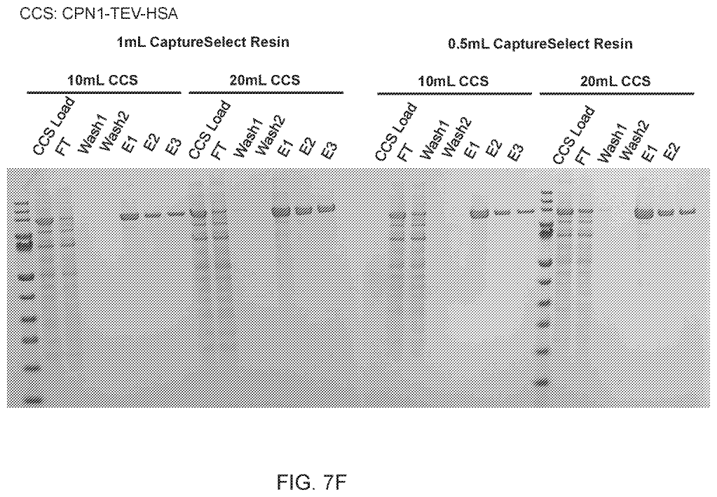

FIGS. 7A-7C depict various chromatography and SDS-PGE results from purification of CPN variants. The CPN variants provided herein can be purified by a generic 2-step purification process negating the need for an affinity purification step with expensive resins. Briefly, clarified culture supernatant (with or without flocculant pretreatment) is diluted 1/10 in Buffer A (50 mM Tris-HCl, 500 mM NaCl pH 7.5) and applied to a Benzamidine-Sepharose column. The column is washed with 4CV of Buffer A to baseline and then eluted with a 20 CV linear gradient from Buffer A to 100% Buffer B (50 mM Tris-HCl, 500 mM NaCl, 1 M Arginine pH 7.5). Peak fractions were pooled corresponding to relative purity on a SDS-PAGE gel (FIG. 7A). Purity was assessed by absolute size exclusion chromatography (aSEC) with major peak purity of 82%, HMWS 5% and LMWS 13%. Pooled fractions were desalted to AEX Buffer A (25 mM Tris-HCl pH 7.5 and loaded onto a AEX column. The column was washed with 4CV of AEX Buffer A to baseline and then eluted with a 30 CV linear gradient. From AEX Buffer A to 100% AEX Buffer B (25 mM Tris-HCl, 1 M NaCl, pH 7.5). Fractions were pooled based o relative purity from a SDS-PAGE gel (FIG. 7B). An increase in purity was increased to 94% main peak fraction and 6% HMWS (FIG. 7C).

### Example 7B: Purification of CPN1 Variants by Affinity Purification

CPN1 constructs may also be purified using affinity purification-based methods. For example, CPN1-HSA (SEQ ID NO: 9) purification and high molecular weight removal was carried out as described below. CPN1-HSA was expressed using the Expi293 expression system, according to manufacturer's recommendations. After expression, cell culture supernatant was harvested and clarified by centrifugation at 3000×g for 30 min followed by vacuum filtration through a 0.22 μm filter.

CaptureSelect human albumin affinity matrix resin (ThermoFisher Scientific, Catalog No 191297050) was equilibrated by incubating the resin slurry with 20 mM Tris, pH 7.4 in a conical bottom centrifuge tube. The mixture was centrifuged at 3000×g for 25 min at room temperature and the supernatant was decanted. This process was repeated 3 times. After equilibration, the resin was loaded with cell culture supernatant, incubated for 30 mins, and centrifuged at 3000×g for 30 mins at room temperature. The supernatant flow-thru was decanted. The resin was then washed 3 times by incubating with 20 mM Tris, pH 7.4, centrifuging at 3000×g for 25 mins and pouring off the supernatant. Finally, the target protein was eluted by incubating the resin with 20 mM Tris, 1 M NaCl, 0.5 M Arg HCl, pH 7.4, spinning down at 3000×g for 10 min at room temperature, and collecting the supernatant. An exemplary SDS-PAGE gel stained with Coomassie showing load, flow-through, and 3 elutions from this procedure using a 500 mL cell culture supernatant load with 25 mL resin slurry is shown in FIG. 7D.

Elution fractions were pooled and dialyzed in 1×PBS and subsequently concentrated to >5 mg/mL using Amicon 10 K 15 mL filters (Millipore Sigma, Catalog No 901024). After concentration, preparative SEC was performed to remove high molecular weight (HMW) species. To perform the preparative SEC, A HiPrep 26/60 Sepharcyl S-200 High Resolution column (Cytiva, Catalog No 17119501) was first equilibrated with 4 column volumes (CV) of 1×PBS. Then, 3 mL concentrated protein sample was loaded via a sample loop onto the column. Separation was performed by running 4 CV 1×PBS over the loaded column. An exemplary chromatogram in FIG. 7E shows a clear separation between HMW (rt˜97 min) and main peak (rt˜118 min). The main peak fractions (rt 113 min-149 min) were pooled and concentrated for endotoxin removal and finishing.

CPN1-TEV-HSA was similarly purified using the CaptureSelect spin column method though at a smaller scale. FIG. 7F shows SDS-PAGE gels stained with Coomassie showing load, flow-through, and 3 elutions at different load and resin volumes.

### Example 8A: C3a and C5a Activity Assay

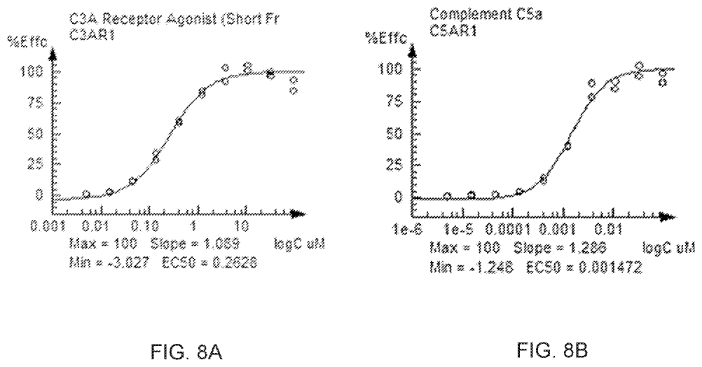

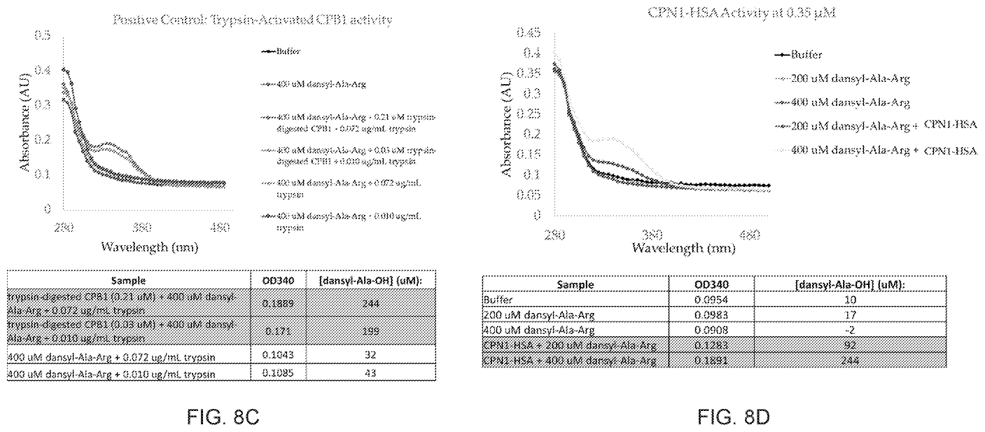

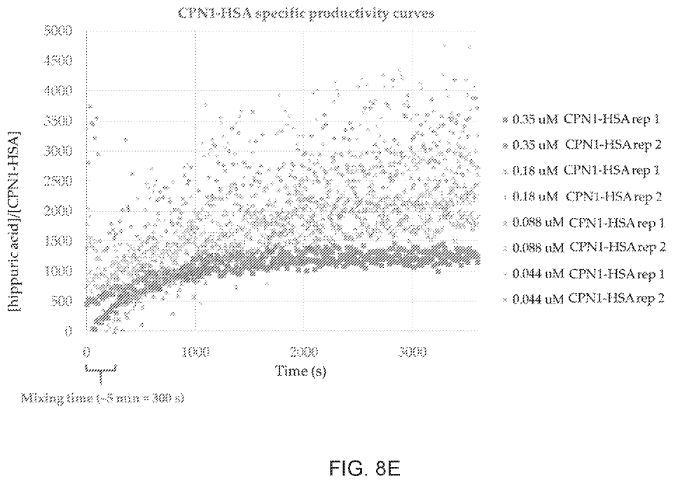

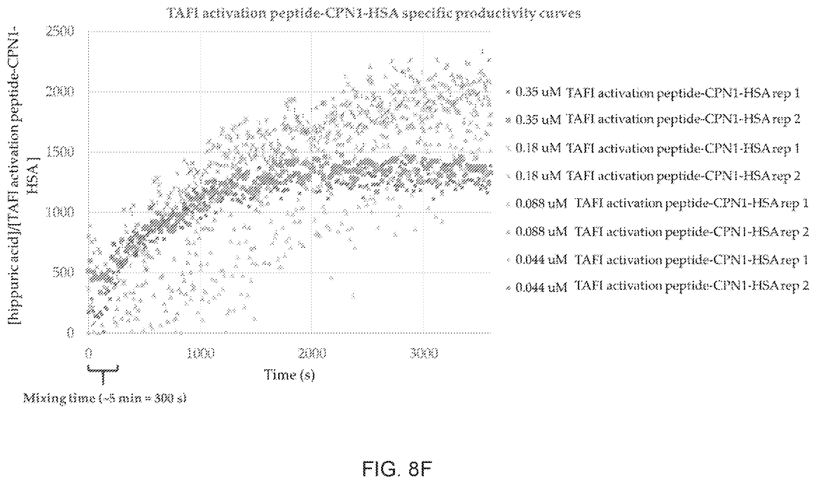

FIGS. 8A-8B depict the results of an activity assay measuring the activation of C3aR and C5aR, respectively. The activity assay is used to examine the activation of C3aR and C5aR by C3a and C5a, and can also be used to screen for CPN activity on C3a and C5a. Briefly, target cells are infected with Retroparticles containing β-Arrestin Enzyme Acceptor (“EA”). Next, the PathHunter β-Arrestin parental cell line is transfected with the GPCR-PK plasmid (containing the GPCR of interest and the β-gal ProLink peptide tag). Next, the ligand to be tested, C3a or C5a, is added. Then, the substrate for active b-Arrestin EA is added. Luminescence is read to measure the amount of residual C3a or C5a and EC₅₀ calculated.

### Example 8B: Activity Characterization Assay

Cell culture supernatants (CPN1-HSA and TAFI activation peptide-CPN1-HSA) or purified protein (CPN1-HSA) were evaluated for carboxypeptidase activity against anaphylatoxin substrates C3a or C5a. CPN1-HSA and TAFI activation peptide-CPN1-HSA amino acid sequences are shown in Table 1.

Carboxypeptidase enzymatic cleavage of terminal arginine from specific substrates (C3a or C5a, Complement Technology, Texas, USA) was assessed by mass spectrometric detection of released arginine. Following in vitro cleavage at 37° C., reactions were stopped via addition of acid (0.4 M perchloric acid, Sigma, Missouri, USA) or specific inhibitors (250 nM 1,10-phenanthroline or EDTA, Sigma, Missouri, USA). Reactions were derivatized (SymDAQ, Charles Rivers Laboratories, California, USA), internal standard (13C6-Arginine, Sigma, Missouri, USA) spiked into reactions and products separated via liquid chromatography-mass spectrometry (LC-MS/MS). Released arginine and spiked calibrator were then measured by mass transition. Released arginine was quantified by linear regression against the spiked in calibrator. Reactant and buffer alone controls were included for background correction.

To further characterize the activity of purified carboxypeptidases cleavage of C3a or C5a anaphylatoxin substrate was assessed using the PathHunter® β-Arrestin assay (Eurofins DiscoveRx, California, USA) for GPCR C3aR1 or C5aR1 cell lines. To provide an approximation for percent cleavage, the cleavage reaction mixtures were run with a full titration curve and compared to native ligand (C3a or C5a) and fully cleaved native ligand (C3a-desArg and C5a-desArg) on their respective receptors. Substrate concentration was varied to generate response curves used to calculate EC50 by nonlinear regression (Prism 9, log (agonist) vs. response-4 parameter variable slope model). Using the calculated EC50's, percent cleavage was estimated by the equation 100%−(native substrate EC50/test article EC50×100). Percent cleavage reported was normalized by subtracting C3a-desArg or C5a-desArg alone background signaling. Native anaphylatoxins and their native-desArg versions, buffer and vehicle alone controls were included for background evaluation.

TABLE 8.1
In vitro carboxypeptidase activity of CPN1-HSA
variants for the anaphyltoxin substrate C3a.

| Variant | Enzyme | Assay | Metric | Measurement |
|---|---|---|---|---|
| CPN1-HSA | Purified | Arg LC-MS/MS | K_{cat}/K_{M} | 8 × 10⁵ M⁻¹ s⁻¹ |
| CPN1-HSA | Purified | Arg LC-MS/MS | EC₅₀ | 4.42 × 10⁻⁹ M |
| CPN1-HSA | Supernatant | Arg LC-MS/MS | % Arg released | 100% |
| TAFI activation | Supernatant | Arg LC-MS/MS | % Arg released | 72% |
| peptide-CPN1-HSA | | | | |
| CPN1-HSA | Purified | GPCR | % Cleavage | 100% |
| Variant | Enzyme | Assay | Metric | Measurement |
| CPN1-HSA | Supernatant | GPCR | % Cleavage | 100% |

TABLE 4
In vitro carboxypeptidase activity of CPN1-HSA
variants for the anaphylotoxin substrate C5a

| Variant | Enzyme | Assay | Metric | Measurement |
|---|---|---|---|---|
| CPN1-HSA | Purified | Arg LC-MS/MS | K_{cat}/K_{M} | ND |
| CPN1-HSA | Purified | Arg LC-MS/MS | EC₅₀ | 3.53 × 10⁻⁸ M |
| CPN1-HSA | Supernatant | Arg LC-MS/MS | % Arg released | 20% |
| TAFI activation | Supernatant | Arg LC-MS/MS | % Arg released | 14% |
| peptide-CPN1-HSA | | | | |
| CPN1-HSA | Purified | GPCR | % Cleavage | 82% |
| CPN1-HSA | Supernatant | GPCR | % Cleavage | 97% |

ND = not determined

### Example 8C: Dansyl-Ala-Arg Activity Assay

Assay principle: CPN1 cleaves C-terminal arginine from dansyl-ala-arg (CAS #: 87687-46-5) substrate. Activity was measured in an end-point assay format. The product dansyl-ala-OH was separated from dansyl-ala-arg after acidification and extraction with chloroform. The dansyl-ala-OH product (CAS #: 53332-27-7) was measured via absorbance at λ=340 nm. Fluorescence of the chloroform-extracted dansyl-ala-OH product can also be measured at λex/λem 340/495 nm.

Stock materials included 10 mM dansyl-ala-arg dissolved in DMSO, 10 mM dansyl-ala-OH dissolved in DMSO for calibration curve, Activity assay buffer: 100 mM Tris, pH 7.4, 50 mM NaCl, 0.01% Tween 80 Quartz cuvette (10 cm path length).

Each reaction mixture (200 μL) was prepared in 1.5 mL Eppendorf tubes and included 400 uM dansyl-ala-arg, activity assay buffer, and 0.35 uM of relevant CPN1 enzyme. The reaction was allowed to proceed at 37° C. for 2 h. To terminate and acidify the reaction, 20 uL of 1 M HCl was added to the reaction tubes. 1 mL of chloroform was then added to each reaction mixture and vortexed for 15 s to extract the product (dansyl-ala-OH) into the chloroform layer. 0.8 mL of chloroform (the bottom phase) was pipetted into the quartz cuvette and absorbance at 340 nm was measured (or scanned for better visualization). The cuvette was rinsed twice with 0.8 mL chloroform between sample measurements. A calibration curve with danysl-ala-OH absorbance at 340 nm was used to quantify amount of product. The substrate dansyl-ala-arg did not extract into chloroform, even at high (2.5 mM) concentrations; in contrast, dansyl-ala-OH provided large signal (λₘₐₓ=340 nm) after extraction. The calibration curve of dansyl-ala-OH was observed to be linear (˜100 μM-1.5 mM).

CPB1 (positive control) and CPN1-HSA were tested using the above protocol. Both CPB1 and CPN1-HSA showed activity as seen in FIG. 8C and FIG. 8D respectively. The corresponding tables in FIG. 8C (for CPB1 positive control) and FIG. 8D (for CPN1-HSA) depict the signal levels and concentrations for CPB1 and CPN1-HSA, respectively.

### Example 8D: Hippuryl-Arg Activity Assay

Assay principle: CPN1 cleaves CPN1 cleaves C-terminal arginine. Both hippuryl-arg (CAS: 744-46-7) and hippuric acid (CAS: 495-69-2) absorb at 254 nm, but hippuric acid has a slightly higher ε. The amount of product produced is quantified by ΔOD254 between sample and [S]₀.

Stocks/Materials included 10 mM hippuryl-arg dissolved in water, 10 mM hippuric acid dissolved in water for calibration curve, activity assay buffer: 100 mM Tris, pH 7.4, 50 mM NaCl, 0.01% Tween 80, and UV-detectable 96-well plates.

2× enzyme solution in a non-binding plate (i.e. 0.7 uM [E]) was prepared. Serial dilutions in this plate were performed when multiple [E] were tested. 50 μL of buffer (for substrate and product standards) or 2× enzyme solution to a UV-detectable plate were added. 2× solutions of substrate and product (i.e. 2 mM hippuryl-arg and 2 mM hippuric acid, respectively) were prepared. 50 μL of substrate and product standard wells were added, followed by 50 μL of substrate to wells with enzyme to start the reaction. The plates were loaded in the plate reader as soon as possible, typically within 20 s of starting the reactions. Absorbance at 254 nm was read in 20 s intervals for 1 h. For analysis the extent of reaction and amount of product produced at each time point was quantified. Mixing effects were seen up to 5 min, so productivity curves (integrated rate equations) were used to fit kinetic parameters. CPB1 was found to generate signal from hippuryl-arg substrate in a dose-dependent manner in above assay protocol.

Activities of CPN1-HSA and TAFI activation peptide-CPN1-HSA were tested using the above assay protocol at various concentrations ranging from 0.044 uM to 0.35 uM, as shown in FIGS. 8E-8F, respectively. In FIG. 8E each concentration of CPN1-HSA was tested in duplicate. Similarly, in FIG. 8F, each concentration of TAFI activation peptide-CPN1-HSA was tested in duplicate.

### Example 9: CPN1-HSA Efficacy after Intravenous Administration in a Rodent Model of Complement Activation

Examination of in vivo activity of CPN1-HSA in Acute Respiratory Distress Syndrome and evaluation of the therapeutic effects of CPN1-HSA and anti-C5 in an LPS-induced acute respiratory distress syndrome (ARDS) mouse model was carried out. The purpose of this study was to assess the efficacy of CPN1-HSA and anti-murine C5 antibody to limit complement mediated acute pulmonary inflammation in a mouse model of ARDS induced by a single administration of lipopolysaccharide (LPS).

Method description: A mouse model of aseptic ARDS was used to study complement involvement following an intratracheal instillation (IT) of LPS. Male C57BL/6 mice (Charles River Laboratories) weighing 20 to 25 g at enrolment were anesthetized under isoflurane and intratracheally instilled with 50 μg LPS (1 mg/mL LPS isolated from E. coli 0111: B4 in 0.9% saline solution, Sigma).

Immediately prior to IT LPS administration, animals received an IV injection of 5 mg/kg CPN1-HSA (as shown in FIG. 12, SEQ ID NO: 9, n=8) or control article (PBST with 0.01% P80; n=8) at a dosing volume of 5 mL/kg. Positive control animals received 1 mg/animal anti-mouse C5 recombinant antibody (Clone BB5.1, Creative Biolabs, n=8) intraperitoneally at a dose volume of 1 mL. This antibody is highly specific for mouse C5 and, like the FDA-approved anti-human C5 monoclonal antibody, eculizumab, BB5.1 binding to C5 efficiently inhibits cleavage of C5 to C5a and C5b. All animals were sacrificed 24 hours post-LPS IT.

Whole body plethysmography was performed prior to LPS instillation, as well as 6- and 24-hours post-instillation. Bronchoalveolar lavage fluid (BALF) was harvested in three 300 μL perfusions of the right lung with cold PBS 1× containing Protease Inhibitor 1× (SigmaFAST®). Evaluation of complement component fragments by mass spectrometry (MS) was performed on K₂-EDTA plasma samples collected at baseline (>5 days prior to study start) and sacrifice as well as lung tissue and BALF samples collected at sacrifice. Cytokine and chemokine levels (Mouse 31 plex Multiplex Immunoassay analyzed with a BioPlex 200 Cytokine Array, Assay Kit Millipore MILLIPLEX, performed by Eve Technologies, Calgary, Canada) were assessed in K₂-EDTA plasma, BALF, and lung tissue (homogenized in PBS 1×+0.1% Triton X-100 with protease cocktail inhibitors) collected at sacrifice and baseline (plasma only). A cell count differential was performed on BALF samples to assess leukocyte recruitment to the lung. Myeloperoxidase (MPO) and histamine levels were assessed in BALF samples by commercially available ELISA kits (Abcam ab155458 and Abcam ab213975).

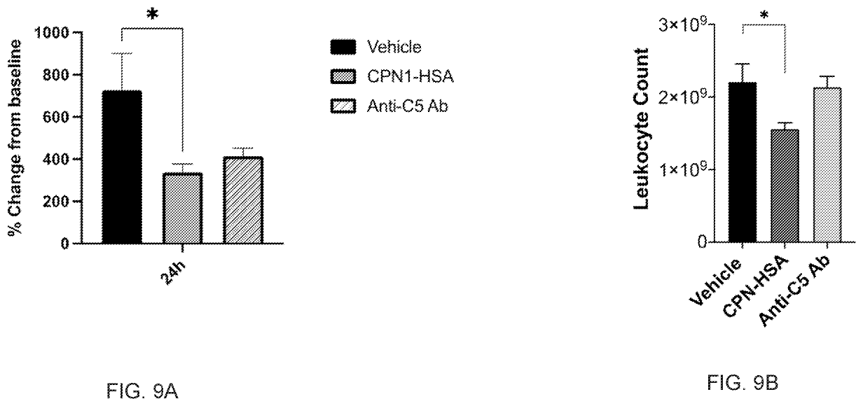

Conclusions: CPN1-HSA administered prophylactically at the time of LPS intratracheal instillation significantly protected against the development of ARDS-like phenotype in mice. PenH, or enhanced respiratory pause, an index of pulmonary congestion associated with ARDS disease severity, increased on average 726% from healthy baseline levels in untreated animals (FIG. 9A). In animals receiving CPN1-HSA this was significantly reduced to an average of 337% greater than baseline (FIG. 9A). A similar but non-significant trend was observed in anti-C5 antibody treated animals. Both treatments demonstrated trends toward protection at 6 hours. The less prominent protection of anti-C5 antibody after 24 hours compared to CPN1-HSA suggests the protease had a longer pharmacodynamic window, possibly due to a longer circulating half-life or increased substrate processing.

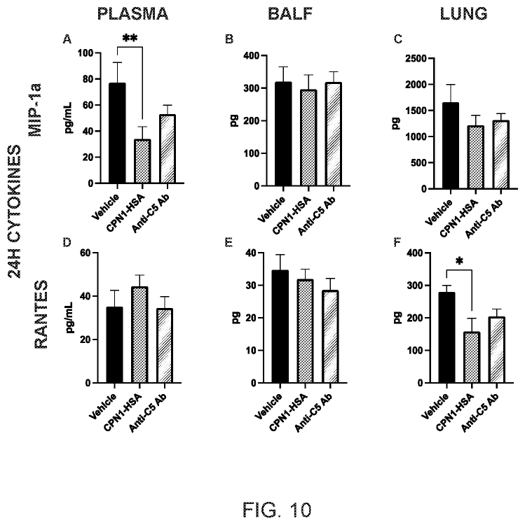

Consistent with the physiologically protective role of CPN1-HSA, administration was associated with significant reductions in pro-inflammatory chemokines in key body compartments at 24 hours (FIGS. 10A-F). Specifically, circulating macrophage inflammatory protein-1 alpha (MIP-1α) was significantly reduced in CPN1-HSA treated animal plasma with slight reductions also observed in the lung but not BALF (FIG. 10A-C). Levels of RANTES (Regulated upon Activation, Normal T Cell Expressed and Presumably Secreted), a proinflammatory multi-functional chemokine secreted by lung epithelial cells, were significantly lower in the lung tissue in CPN1-HSA treated animals compared to untreated controls with a similar non-significant trend observed in anti-C5 antibody treated animals (FIG. 10F). These effects were not observed in plasma levels, consistent with the epithelial surface role of this chemokine in lung injury (FIG. 10D). Mice engineered to constitutively overexpress RANTES demonstrate increased neutrophil migration to the airways. In mice, deletion of CCR1, a receptor that detects MIP-la and RANTES, is associated with protection from inflammatory lung injury secondary to pancreatitis. The ability to modulate this signaling pathway speaks to the potential role of CPN1-HSA to similarly regulate other inflammatory pathologies due to complement dysregulation.

The findings demonstrated in this model confirm that CPN1-HSA administration is as efficacious, if not more, than anti-C5 antibody treatment to protect against LPS-induced development of ARDS in mice. The number of molecules of CPN1-HSA administered required to produce a comparable or superior effect was over 7-fold less compared to anti-C5 antibody dosing (Table 5, columns C and D). The assumptions for this calculation are described in Table 5 columns A and B below.

TABLE 9.1
In vivo study parameters

| | | | | D |
|---|---|---|---|---|
| | A | | | Dose Level Fold- |
| | Molecular | B | C | Difference |
| | Weight | Dose | Dose | (Anti-C5 |
| Treatment | (g/mol) | (mg/kg) | μmol/kg | Ab:CPN1-HSA) |
| CPN1-HSA | 112,700 | 5.0 | 0.04 | 7.25 |
| Anti-C5 Antibody | 150,000^{A} | 44.05^{B} | 0.29 | |

^{A}Molecular weight estimated based on standard murine IgG;
^{B}Dose level calculated using the mean body weight of anti-C5 treated animals at time of dosing (22.7 g)

### Example 10: In Vivo Pharmacokinetics Assays in Rat Model

Examination of in vivo pharmacokinetics of CPN1-HSA was carried out in rats. The pharmacokinetics profile of pooled CPN1-HSA construct numbers 351 and 6 (the only difference being the presence of an 8 or 12 linker-length, respectively) was assessed following a single intravenous injection (IV) in adult male and female Sprague Dawley rats, four each for a total of eight animals. Animals were dosed at 2 mg/kg intravenously via tail vein injection. Blood samples were collected into EDTA pre-dose and at 0.5 h, 1 h, 3 h, 6 h, 12 h, 24 h, 48 h, 72 h, 120 h, 168 h, 240 h, and 336 h followed by plasma separation for PK bioassay analysis.

CPN1-HSA concentration in plasma was assessed via a quantitative sandwich enzyme electrochemiluminescence (ECL) antigen assay for detection of construct numbers 351 and 6 in rat EDTA plasma. For quantification, Meso Scale Discovery (MSD, Rockville, MA) assay plates were prepared by coating with 1 μg/ml of immunogen purified rabbit polyclonal capture antibody specific to the antigen (MyBioSource, San Diego, CA). Standards, QCs, and plasma samples were added to the plate wells and incubated to facilitate capture. Plates were washed followed by addition of 1 μg/ml of a biotinylated human HSA monoclonal antibody (Invitrogen, Waltham, MA) and 0.3 μg/ml SULFO-TAG Streptavidin (MSD, Rockville, MD) for detection. The plates were once again washed and MSD Read Buffer T (2×) added. Plates were read using a MSD Sector S 600 reporting ECL relative light units (RLU).

CPN1-HSA plasma concentrations were interpolated via standard curve and pharmacokinetic (PK) parameters derived from a noncompartmental analysis performed in Excel based on the determined concentrations. Area Under the Curve (AUC) was calculated using the linear trapezoidal method, AUC 0-inf was calculated from the AUC 0-t (determined up to the last measurable concentration) and then extrapolated to infinity using the estimated half-life. Mean Residence Time (MRT) was calculated using the moment theory method (Area Under the Moment Curve [AUMC 0-inf] divided by AUC 0-inf). Sufficient sampling was determined by confirming that the extrapolated AUC did not exceed 20% of total measurable AUC.

For PK parameter estimates, three subjects were included in the analysis. Of the eight total animals, one was excluded due to inconsistently high baseline detection compared to the other animals and four animals did not meet model criteria due to too few datapoints above assay Lower Limit Of Quantitation (LLOQ). Of note, a sample collected immediately post-dose was not obtained. Given the immediate and complete absorption of an IV dose into circulation, the Cₘₐₓ is likely under-estimated and the Tₘₐₓ over-estimated for this report. PK parameter estimates from the three animals assessed are summarized in FIG. 11A. The PK profiles of the 3 animals analyzed are depicted in FIG. 11B, and the representative PK profile is depicted in FIG. 11C.

The pharmacokinetic profile of CPN1-HSA is determined following a single subcutaneous injection in Sprague Dawley rats. Adult male and female rats weighing between 200 g and 250 g at the time are used and pair-housed during the acclimation period and experimental phase of the study. Prior to the study, a blood sample is taken for pre-dose measurement a minimum of one day prior to dosing.

Animals are injected subcutaneously with test compounds. Experimental groups are dosed at 6 mg/kg of CPN1-HSA subcutaneously. Subcutaneous doses are administered via bolus injection between the skin and underlying layers of tissue in the scapular region on the back of each animal.

### Example 11: In Vivo Pharmacokinetics Assays in Primate Model

The pharmacokinetics profile of CPN1-HSA are determined following a single intravenous injection or single subcutaneous injection in cynomolgus macaques. Adult male and female macaques age 2-4 years at study start and weighing between 2-4 kg at study start are used.

On study day 0, the test article is delivered by a single intravenous bolus or a single subcutaneous injection to alert, chair-restrained adult male and female macaques. Experimental groups are dosed at either 2 mg/kg of CPN1-HSA intravenously or 6 mg/kg of CPN1-HSA subcutaneously. FIG. 12, Table 1 summarizes the dose levels and regimen of the test articles and vehicles.

Test articles and vehicles are administered by single intravenous or subcutaneous injection. For intravenous doses, 2 mg/kg of CPN1-HSA are administered as a bolus via catheter followed by approximately 5 mL sterile saline flush. The dose time is recorded at the time the saline flush is completed. For subcutaneous doses, 6 mg/kg of CPN1-HSA are administered to alert, chair-restrained animals by injection between shoulder blades.

For both intravenous and subcutaneous administration, baseline blood is collected immediately prior to dosing. Blood collections are performed in alert, chair-restrained animals. For animals dosed intravenously, blood is collected from the leg opposite the intravenous dosing site. FIG. 12, Table 2 summarizes the blood collection schedule.

Samples for plasma isolation are collected into tubes containing K₂-EDTA until processing. Samples are centrifuged at 2000×g for 10 minutes at 4° C. within 1 hour of collection. The presence of hemolysis is documented following centrifugation, and the maximum amount of plasma is recovered and frozen prior to analysis.

Samples for serum isolation are collected into serum separator tubes, stored at ambient temperature for at least 15 minutes or until blood is clotted, and then processed. Samples are centrifuged at 2000×g for 10 minutes at 4° C. within 1 hour of collection. The presence of hemolysis is documented following centrifugation, and the maximum amount of serum is recovered and frozen prior to analysis.

### Example 12: Serum Stability Assays

Serum stability was measured for CPN1-HSA constructs at 37° C. for extended times. To quantify protein levels, semi-quantitative Western blots were performed with CPN1 primary antibodies. Constructs were dosed into serum and diluted 1:50, diluted in LDS, reduced with a reducing agent, and boiled for 5 minutes.

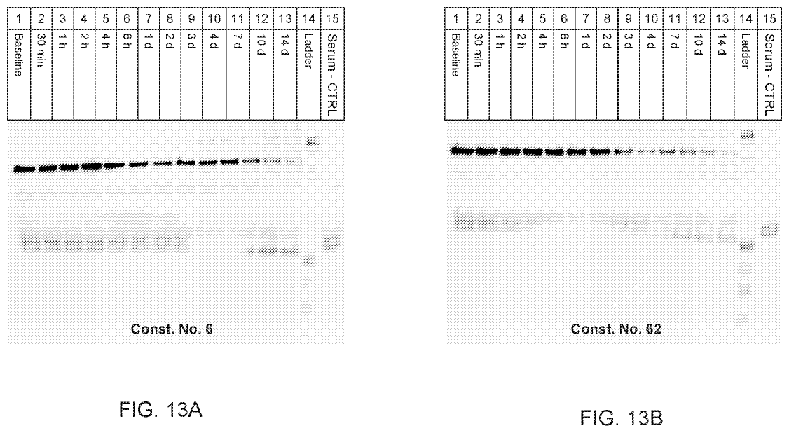

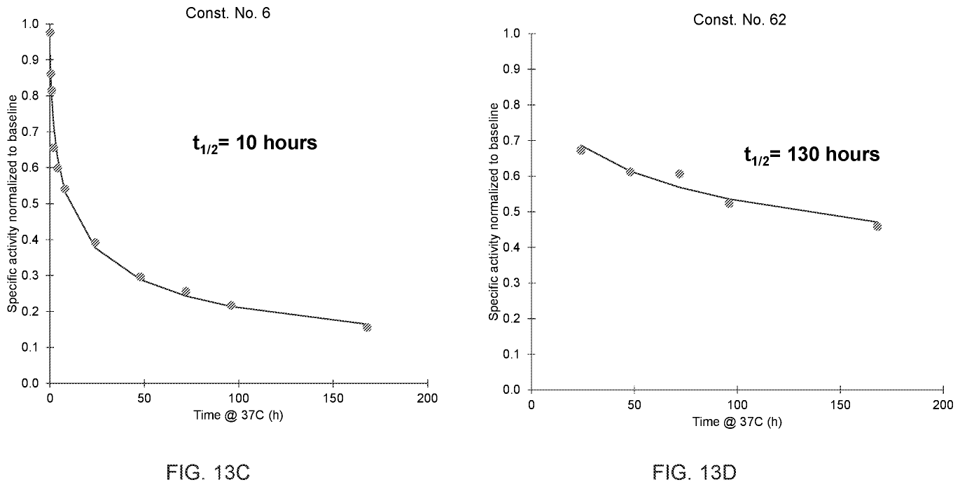

For the Western blot, 15 μL of samples were loaded into each well, and the gel run at 100V for 10 minutes, then 150V for 90 minutes. Afterwards, the gel was transferred to a nitrocellulose membrane and blocked for 1 hour in 5% nfd milk at room temperature. Then, the blot was incubated with CPN1 primary antibodies at 1:1000 dilution in 5% nfd milk overnight at 4° C. The following day, the blot was subjected to three 10-minute TBST washes, then incubated with secondary antibody at 1:20000 dilution in 5% nfd milk for 1 hour at room temperature. Afterwards, the blot was again washed three times for 10 minutes with TBST. Lastly, the blot was developed and imaged with SuperSignal™ West Pico PLUS Chemiluminescent Substrate. FIGS. 13A and 13B depict Western blots for two exemplary CPN1-HSA constructs.

Using the semi-quantitative Western blot, a half-life curve was generated for CPN1-HSA constructs. FIGS. 13C and 13D depict half-life curves for exemplary CPN1-HSA constructs in serum.

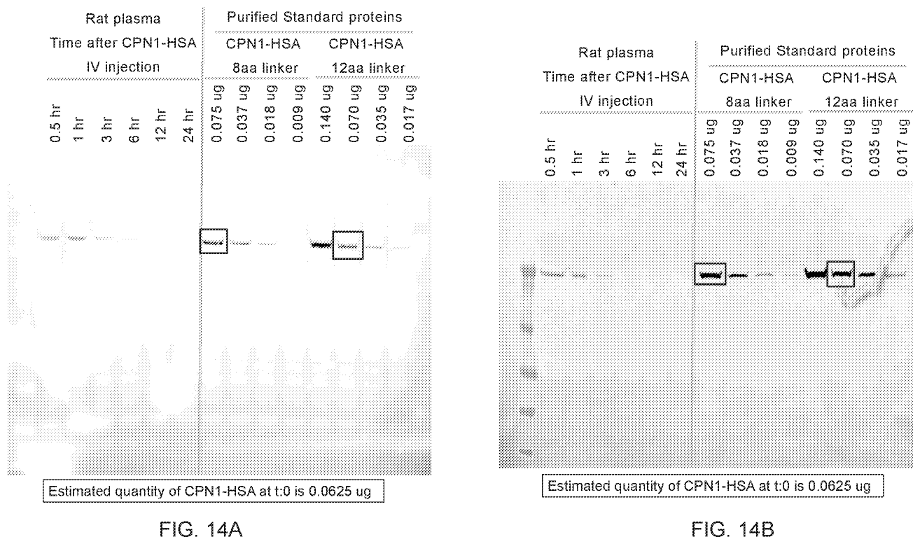

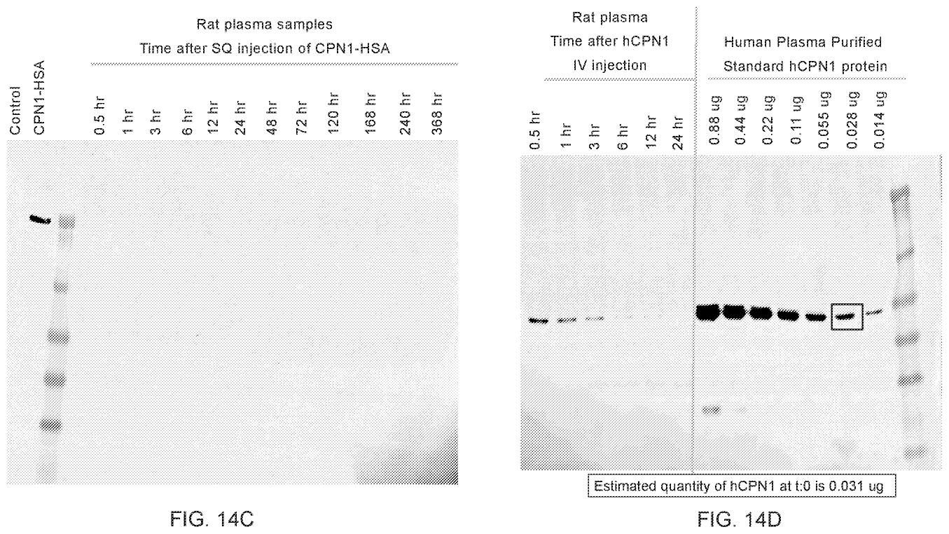

Western blot analysis was also performed on plasma isolated from rats following intravenous or subcutaneous injection of CPN1-HSA constructs. FIGS. 14A and 14B depict two different rats' plasma samples and the presence of two exemplary CPN1-HSA constructs. Western blot analysis was then performed on plasma isolated from rats following subcutaneous injection of CPN1-HSA constructs, and is depicted in FIG. 14C. Lastly, Western blot analysis was performed on plasma derived from rats subjected to intravenous injection of human plasma purified CPN1, and is depicted in FIG. 14D.

### Example 13: Protease Activity Assay

The relative activity of CPN constructs was measured via modified use of the Pefakit TAFI (thrombin activatable fibrinolysis inhibitor) assay, which determines the activity of CPN proteins on a synthetic substrate relative to a TAFI-containing plasma calibration curve. The assay provides no information on activity or enzyme kinetics of CPN proteins on native substrates, but serves as a high-throughput method for determining relative activities o CPN proteins.

Enzymatic hydrolysis of the synthetic substrate CPN protein generates colorimetric 5-mercapto-2-nitro-benzoic acid product. The rate of increase in absorbance is interpolated against a plasma calibration curve to yield the % TAFIa (thrombin activatable fibrinolysis inhibitor activated form) activity.

The activity was calculated in two ways. First, the product generation rate was interpolated from the plasma calibration curve and dilution corrected to yield a volumetric activity in units of % TAFIa activity. The volumetric % TAFIa activity indicates the amount of calibrated TAFI-containing plasma required to achieve activity equal to the sample. This is useful for comparing samples with equal concentrations. The second activity was calculated by dividing the % TAFIa activity of the samples by the sample enzyme concentration to yield a specific activity in units of % TAFIa activity per μM enzyme (/μM). The specific % TAFIa activity indicates the intrinsic activity of the CP protein in the sample regardless of the concentration. For example, if multiple samples have equally active CP protein at different concentrations, the volumetric activities will differ while the specific activities would not.

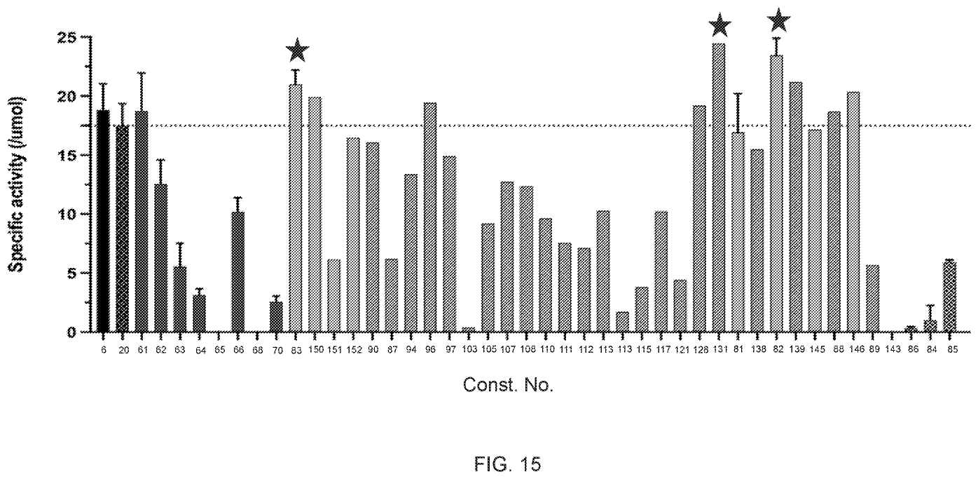

The specific activity of CPN constructs was measured and compiled into a graph depicted in FIG. 15. The bars marked with asterisks indicate improved performance over Const. No. 6.

### Example 14: In Silico Design of CPN Constructs

CPN1 was reengineered based on structural understanding of the enzyme from crystallographic data of human CPN1 (PDB ID: 2 nsm) and homologous carboxypeptidases. A number of computational tools were used in the development of designs aimed at improving and modulating a number of CPN1 properties including stability, solubility, activity, and substrate selectivity. The primary tool used in these designs was the Rosetta suite of programs in addition to the docking program PIPER and molecular dynamics simulations via OpenMM.

A computational site-saturation mutagenesis experiment was run to identify positions in the native CPN1 structure that could be modified in order to improve stability. Clusters of amino acids around the identified hot spots were mutated for improved stability using the FastDesign algorithm of Rosetta. Molecular dynamics simulations of select mutations revealed further sites of instability which were rectified via the introduction of strategic disulfide bonds. Additionally, a workflow involving docking, molecular dynamics simulations, and homology modeling was used to determine the binding interaction between CPN1 and the complements C3a and C5a. These complexes served as the basis for CPN1 mutations that aimed at improving selectivity for either C3a or C5a. Further docking experiments with endogenous CPN1 substrates was used to identify potential prodomains that could be fused to the N-terminus of CPN1 with a cleavable linker to create a zymogen out of the enzyme, using the same cleavage site (SRVR|AT, SEQ ID NO: 72) found in thrombin activatable fibrinolysis inhibitor (TAFI).

1. A variant of a carboxypeptidase N catalytic subunit 1 (CPN1 variant) comprising at least one modification with respect to a wild type CPN1, wherein the CPN1 variant has at least one improved characteristic as compared to the wild type CPN1.

2. The CPN1 variant of claim 1, wherein the modification with respect to a wild type CPN1 comprises any one or more of: a substitution of one or more amino acid residues, a deletion of one or more amino acid residues, an insertion of one or more amino acid residues, an insertion of one or more CPN domains, and an insertion of one or more non-CPN domains or components.

3. The CPN1 variant of claim 1, wherein the at least one improved characteristic is selected from an increase or a decrease in any one or more of: half-life, activity, potency, substrate affinity, substrate specificity, substrate selectivity, proteolytic sensitivity, cofactor affinity, and catalytic capability.

4. The CPN1 variant of claim 3, wherein the at least one improved characteristic comprises an increase in affinity for one or more substrates, and wherein at least one substrate is C3a.

5. The CPN1 variant of claim 1, wherein the at least one improved characteristic comprises:
(a) an increase in affinity for one or more substrates, and wherein at least one substrate is C5a;
(b) an increase in the cleavage of C3a and/or C5a;
(c) an increased k_{cat}/K_{M}(M⁻¹ s⁻¹) for cleavage of C3a and/or C5a;
(d) a decreased K_{D} (nM) value for cleavage of C3a and/or C5a;
(e) a decreased EC₅₀ (nM) value for cleavage of C3a and/or C5a, compared to the wild type CPN1;
(f) increased half-life observed in plasma; or
(g) a combination of (a)-(f).

6-16. (canceled)

17. The CPN1 variant of claim 1, wherein the CPN1 variant comprises at least one modification corresponding to a wild type non-human CPN1.

18. The CPN1 variant of claim 1, wherein the CPN1 variant comprises at least one modification corresponding to a wild type human CPN1.

19. The CPN1 variant of claim 18, wherein the CPN1 variant comprises at least one modification corresponding to a wild type CPN1 comprising the amino acid sequence as set forth in SEQ ID NO: 6.

20. The CPN1 variant of claim 1, wherein the CPN1 variant comprises an amino acid sequence having at least 70% sequence identity to SEQ ID NO: 6.

21. The CPN1 variant of claim 1, wherein the CPN1 variant comprises the amino acid sequence as set forth in any one of SEQ ID NOs: 7-8 and 11-12, or an amino acid sequence having at least 70% sequence identity to any one of SEQ ID NOs: 7-8 and 11-12.

22-24. (canceled)

25. The CPN1 variant of claim 1, wherein the CPN1 variant comprises the amino acid sequence of SEQ ID NO: 6, SEQ ID NO: 7, SEQ ID NO: 8, SEQ ID NO: 11, or SEQ ID NO: 12, wherein the amino acid sequence further comprises a modification string, and wherein the modification string is selected from the group consisting of the modification strings provided in Table 3A, Table 3B, Table 3C, Table 4A, Table 4B, and Table 4C.

26. (canceled)

27. A fusion construct comprising a carboxypeptidase N catalytic subunit (CPN1) or variant thereof.

28. The fusion construct of claim 27, comprising an amino acid sequence selected from the group consisting of SEQ ID NO: 73-SEQ ID NO: 485.

29. The fusion construct of claim 27, wherein the fusion construct comprises the amino acid sequence of SEQ ID NO: 6, or an amino acid sequence having at least 70% sequence identity to SEQ ID NO: 6.

30. (canceled)

31. The fusion construct of claim 27, wherein the CPN1 variant comprises at least one modification with respect to a wild type CPN1, wherein the CPN1 variant has at least one improved characteristic as compared to the wild type CPN1.

32. The fusion construct of claim 27, wherein the CPN1 variant comprises at least one modification corresponding to a wild type CPN1 comprising the amino acid sequence as set forth in SEQ ID NO: 6.

33. The fusion construct of claim 27, wherein the CPN1 variant comprises the amino acid sequence as set forth in any one of SEQ ID NOs: 7-8 and 11-12, or an amino acid sequence having at least 70% sequence identity to any one of SEQ ID NOs: 7-8 and 11-12.

34-36. (canceled)

37. The fusion construct of claim 27, wherein the CPN1 variant comprises the amino acid sequence of SEQ ID NO: 6, SEQ ID NO: 7, SEQ ID NO: 8, SEQ ID NO: 11, or SEQ ID NO: 12, wherein the amino acid sequence further comprises a modification string, and wherein the modification string is selected from the group consisting of the modifications provided in Table 3A, Table 3B, Table 3C, Table 4A, Table 4B, and Table 4C.

38. (canceled)

39. The fusion construct of claim 27, wherein the fusion construct comprises:
(a) a Glutathione S transferase (GST) amino acid sequence;
(b) a mammalian maltose binding protein (mMBP) amino acid sequence;
(c) a small ubiquitin modifying enzyme (SUMO) amino acid sequence;
(d) a Tobacco Etch Virus protease cleavage site (TEV) amino acid sequence;
(e) an activation peptide of CBP2 (the N-terminal 96aa of a CBP2 protease) amino acid sequence;
(f) a Factor Xa protease cleavage site (Xa) amino acid sequence;
(g) a portion of a regulatory CPN2 subunit amino acid sequence;
(h) a CD180 amino acid sequence;
(i) a LR1G1 amino acid sequence;
(i) at least one non-CPN1 or non-CPN2 domain or component:
(k) an activation peptide that increases sensitivity of the fusion construct; or
(l) a half-life extender.

40-63. (canceled)

64. The fusion construct of claim 27, wherein the fusion construct is non-immunogenic, is in a zymogen form, and/or is in an active form.

65-66. (canceled)

67. A method of treating a disease or condition in a subject in need thereof, comprising administering to the subject the CPN1 variant of claim 1.

68-76. (canceled)

77. A nucleic acid encoding the CPN1 variant of claim 1.

78. A pharmaceutical composition comprising the CPN1 variant of claim 1, and optionally a pharmaceutically acceptable carrier.

79. A method of treating a disease or condition in a subject in need thereof, comprising administering to the subject the fusion construct of claim 27.
